(19) United States
(12) Patent Application Publication
(10) Pub. No.: US 2026/0207626 A1
(43) Pub. Date: Jul. 23, 2026

(54) SYNTHETIC TRITERPENOIDS AND COMPOSITIONS THEREOF IN WEIGHT MANAGEMENT, SKELETAL MUSCLE FUNCTION, AND APPETITE SUPPRESSION

(71) Applicant: The Board of Regents of the University of Texas System, Austin, TX (US)

(72) Inventors: Sara M. Reyna, Edinburg, TX (US); Shizue Mito, McAllen, TX (US); Phoebe Fang-Mei Chang, San Antonio, TX (US)

(21) Appl. No.: 19/138,566

(22) PCT Filed: Dec. 28, 2023
(86) PCT No.: PCT/US2023/086275

§ 371 Date: Jun. 12, 2025

Related U.S. Application Data
(60) Provisional application No. 63/436,097, filed on Dec. 29, 2022.

(51) Int. Cl. A61K 31/575; A61K 9/00; A61P 3/04

(52) U.S. Cl. CPC A61K 31/575; A61K 9/0053; A61P 3/04

(57) ABSTRACT

CDDO and CDDO derivatives for improving glucose transport in skeletal muscle, blocking fat accumulation, inhibiting obesity, maintaining a healthy body weight and inhibiting appetite (an appetite suppressant) are provided. Dietary supplements, additives, and food materials (diet bars, diet supplements) comprising CDDO-EA and/or other CDDO-derivatives, as well as processed food products, high fat food additive products (e.g., lard), veterinary animal feed/chow products, specialty animal foods (advanced age formulas, etc.), animal supplements, foods/formulations that comprise CDDO and/or derivatives, are also disclosed. Preparations/methods for inhibiting LPS mediated secretion of chemokines and cytokines, as well as methods for blocking NF-κB, are also presented.

### CROSS-REFERENCE TO RELATED APPLICATIONS

This application is a National Stage of International Application No. PCT/US2023/086275, filed on Dec. 28, 2023, which claims the benefit of U.S. provisional patent application No. 63/436,097, filed Dec. 29, 2022, the contents of which are incorporated herein by reference in their entireties.

### GOVERNMENT LICENSE RIGHTS

This invention was made with government support under NIH (National Institutes of Health) Grant SC2GM127272 awarded by NIGMS. The government has certain rights in the invention.

### FIELD OF THE INVENTION

The present invention relates to the fields of pharmacologically active preparations, methods and foods for weight management, skeletal muscle function and appetite suppression.

### BACKGROUND OF THE INVENTION

Naturally occurring triterpenoids (TP) in plants are used for medicinal purposes in many Asian countries. Some ursolic and oleanolic acids (OAs) are reported to be anti-inflammatory and anti-carcinogenic (Huang et al., 1994; Nishino et al., 1988). However, the biological activity of these naturally-occurring molecules is relatively weak.

Efforts to create oleanolic and ursolic acid analogs/derivatives with improved anti-inflammatory and antiproliferative activity have resulted in the creation of a family of synthetic triterpenoids, 2-cyano-3,12-dioxooleane-1,9 (11)-dien-28-oic acids (CDDOs, RTA 402), having modifications at the C17 position, (methyl ester (CDDO-Me), ethyl amide (CDDO-EA) and imidazole (CDDO-Im)). These CDDO analogs have been reported to possess widely variable and different pharmacological activities, depending on the target cell and tissue type (Honda et al., 1997; Honda et al., 1998; Honda et al., 2000; Honda et al., 2002; Honda et al., 2004). The CDDO-Me analog prepared according to conventional methods (Honda et al, 2007), has been reported to have some application in smooth muscle tissue, such as in kidney/renal tissue (U.S. Pat. No. 8,129,429). Stack et al. (2010) examined CDDO-EA and CDDO-TFEA in a Huntington's disease animal model (neurodegenerative disease) and reported that oxidative stress in skeletal muscle was reduced, and that CDDO-EA and CDDO-TFEA accumulated in the skeletal muscle.

Two particular TP analogs, CDDO-Me and CDDO-Im, have been reported to possess the ability to modulate transforming growth factor-β (TGF-β)/Smad signaling in several types of cells (Suh et al., 2003; Minns et al., 2004; Mix et al., 2004). Both of these TP analogs are described as potent inducers of heme-oxygenase-1 and Nrf2/ARE signaling (Liby et al., 2005). A series of other synthetic triterpenoid (TP) analogs of oleanolic acid have been described as potent inducers of the phase 2 response, that results in an elevation of NAD (P) H-quinone oxidoreductase and heme oxygenase 1 (HO-1). Thus, some TP analogs have been described as using the antioxidant response element-Nrf2-Keap1 signaling pathway.

The role of TP analogs in the inflammatory response process in skeletal muscle has not been explored. Inflammation and glucose transport in skeletal muscle are considered important physiological processes in insulin induced glucose uptake; and as important in the maintenance of normal skeletal muscle function.

Synthesis protocols to prepare TP analogs with higher bioactivity, from oleanolic acid, have been described by several groups (e.g., CDDO-MA, CDDO-Me, CDDO-EA, CDDO-Im; See Honda et al., 1997, 1998, 1999, 2000a, 2000b, 2002; Suh et al., 1998; 1999; 2003; Place et al., 2003; Liby et al., 2005). While these TP analog synthesis protocols appeared promising, they required extremally complicated/multi-step processes (Yates et al.)), and were prohibitively time intensive (24 hours), rendering these less suitable for manufacture in commercial, scalable yields. Fu et al. (2013) reports a multi-step synthesis for bardoxolone methyl (CDDO-Me) from oleanolic acid, as a scalable alternative approach to synthesizing CDDO-Me. However, the process has been found to produce reaction mixtures having unintended impurities and/or failing to yield appropriate intermediate compounds. Overall, current methods result in high costs for synthetic CDDO compounds, despite their promising applications. The chemical arts remain in need of an alternative and improved synthesis strategy for producing pharmacologically active triterpenoid derivatives, in a scalable, efficient, and cost-effective manner.

Weight management and the incidence of obesity continue to present significant populational health challenges and have been linked to impaired skeletal muscle health (including myosteatosis), overall metabolic syndrome (defined by a cluster of conditions including hypertension, high blood glucose levels, and high serum triglycerides levels) and type 2 diabetes (T2D). A low calorie/low fat diet and exercise remain the intervention strategies of choice in maintaining a healthy weight and preventing obesity and T2D. The incidence of obesity/T2D worldwide continues to increase, and diets higher in fat remain the more commonly available option for most of the population. A higher incidence of obesity has also been linked to consuming a diet higher in fat content (at least 50% of total calories from fat) compared to a lower fat (between about 10% to about 20% of total calories from fat). The increased incidence of obesity in the population, and with it the attendant health risks and challenges of chronic inflammation, creates an even greater risk of developing T2D, and with this, a higher cost of health care in a growing segment of the population.

Skeletal muscle plays a pivotal role in insulin-stimulated whole-body glucose disposal. Skeletal muscle is also recognized as the major site of insulin-induced glucose uptake. Inflammation in skeletal muscle results in dysregulation of normal glucose metabolism in the muscle and can result in development of insulin insensitivity of skeletal muscle tissue. Obesity results in chronic inflammation of skeletal muscle tissue and the development of obesity-induced insulin resistance in skeletal muscle. These physiological series of events often progress to the development of T2D. Therefore, methods for inhibiting chronic inflammation in skeletal muscle would provide an approach to preventing or retarding the incidence of T2D and reducing development of obesity-induced insulin resistance in obese subjects.

A need remains in the medical arts for preparations and weight management methods useful for healthy weight maintenance, metabolically and physiologically tolerated higher fat foods/diets, inhibiting, preventing and/or blocking development of obesity or risk of obesity, maintaining normal/moderate blood glucose and insulin levels, and controlling chronic muscle inflammation. A need also remains in the chemical arts for consistent and efficient methods for a more economically feasible and scalable method for synthesizing useful compounds for these and other purposes, including triterpenoid compounds. These triterpenoid compounds include, for example, CDDO, CDDO-Me, CDDO-EA, and other CDDO analogs and derivatives.

### SUMMARY OF THE INVENTION

The above and other needs in the medical and health care arts are met with the materials and methods presented in the present disclosure.

Novel methods of preparing and using CDDO compounds (CDDO-EA, CDDO-Me, CDDO-MA, CDDO-Im, CDDO-TFEA, or any combination thereof) to restore normal glucose transport in skeletal muscle are presented. Methods of preventing and/or inhibiting weight gain/obesity, especially in subjects consuming a high fat diet, are described, and include the administration of one or more of the CDDO compounds (CDDO-EA, CDDO-Me, CDDO-MA, CDDO-Im, CDDO-TFEA) to a subject, especially a subject identified as being overweight, obese, or having a high risk of becoming obese.

As used in the description of the present technology, the term “obese” is used to describe a condition in a subject (human or veterinary) defined by excessive body fat, specifically a Body Mass Index (BMI) of greater than or about 25 toa BMI of about 30. This metric of about 25 to about 30 BMI is used in both children and adults. In children, an “obese” condition may be identified in children that are as young as 6, 7 or 8 years old. Children, particularly those between 7 years and about 12 years old, may be managed safely with the present preparations to prevent weight gain and/or to reduce body weight without significant risk of harmful side effects. The present technologies in feeding and in food preparation and/or supplements provide a heath management tool easily incorporated into routine dietary plans.

Obesity, and being overweight in general, is a chronic disease that increases the risk of various health problems, including heart disease, type 2 diabetes, and certain cancers. BMI is calculated by dividing weight in kilograms by height in meters squared. BMI between at least about 18.5 BMI to about 24.9 BMI is generally considered to be a healthy weight. BMI of about 25 to about 29.5 is considered to be overweight, borderline obese on the upper end of the range. A subject BMI of about 30 is considered to indicate an obese condition. A BMI of about 40 or greater is generally considered to be morbidly obese. A BMI of less than about 18.5 or less is considered to be underweight.

Preparations, food additives, supplements and health management programs with the present preparations, are also presented. These may be used alone or together with existing drugs, foods, systems and techniques for preventing and/or treating obesity, as well as for managing subjects and in preventing a subject from becoming overweight.

Non-veterinary and veterinary therapeutic agents, pharmaceutical formulations and diet management preparations are also provided as a component of methods for maintaining normal and/or healthy weight in a subject. Methods for protecting a subject against and/or inhibiting/reducing skeletal muscle inflammation, are also presented.

Methods for inhibiting and/or reducing chronic inflammation of skeletal muscle tissue are also disclosed.

In yet another aspect, methods for inhibiting nuclear factor kappa B (NF-κB) activation in skeletal muscle are provided. These methods are particularly useful in non-diabetic (not T2D, not TID) subjects. Methods for preventing and/or retarding skeletal muscle inflammation are also disclosed.

Other aspects of the methods provide for inhibiting cytokine and chemokine gene expression and cytokine and chemokine production in skeletal muscle. For example, the methods for inhibiting gene expression of cytokines (TNF-α, IL-1B, IL-6) and/or chemokines (MCP-1), by skeletal muscle are provided, and include the herein described CDDO and CDDO analogs and derivatives, including any one or combination of CDDO-EA, CDDO-Me, CDDO-Im, CDDO-MA, and/or CDDO-TFEA.

In yet another aspect, methods and preparations for preventing and/or inhibiting excessive weight gain and/or obesity in a subject, including both human (adult and children) and veterinary subjects, are provided. In addition, methods for regulating body weight and for weight management in general, by providing particular food preparations and/or dietary supplements that comprise a CDDO or CDDO analog (CDDO-EA, for example), are presented. In yet another aspect, an appetite suppressant is provided comprising a synthetic CDDO analog or derivative, or combination of these, including any one or more combinations of CDDO-EA, CDDO-Me, CDDO-Im, CDDO-MA, and/or CDDO-TFEA.

In particular aspects, the active ingredient in the compositions, food preparations, and pharmaceutical preparations, provided as part of the methods described herein, comprise a synthetic CDDO or a CDDO analog, including any one or more combinations of CDDO-EA, CDDO-Me, CDDO-Im, CDDO-MA, and CDDO-TFEA.

An example of a particular CDDO composition may be described as comprising an amount of CDDO-EA, CDDO-Me, CDDO-MA, CDDO-Im, CDDO-TFEA, or any combination of two or more of these. The preparations and compositions, in some embodiments, comprise a CDDO composition comprising a particular CDDO compound, such as CDDO-EA (that is essentially free of any other CDDO species/analog/derivative). In other embodiments, the CDDO composition comprises two or more CDDO analogs and/or derivatives as an active ingredient. The CDDO compositions may be formulated as a component of a veterinary or human nutritional and/or food supplement, meal replacement, food additive, combined food and/or nutrient product, weight loss product, appetite suppressant, or other nutritional or other subject health support preparation. The amount of the CDDO component in any particular preparation will be determined according to the needs of the particular subject, and the application and purpose of the preparation.

In general, the amount of the CDDO component may be described as a fat-inhibiting concentration. The food preparations and/or dietary supplements may be a fluid (such as a product suitable to drinking as a beverage) or solid (powder, solid bar, cereal) (e.g., animal chow feed, nutritional bar, cookie, meal substitute material, or other food product, particularly a food product considered high in fat content).

A “high fat” content diet is defined for purposes of the present disclosure as a diet food, food or dietary regimen that comprises greater than an about 20% to about 80% fat caloric content, about 25%, about 30%, about 35%, about 40%, about 45%, about 50%, about 60%, about 70%, about 75%, about 80%, about 85%, about 90%, about 95%, about 20% to about 80% or any higher percentage of total calories from fat (100%). A “low fat” content diet is defined for purposes of the present disclosure as less than about 5%, about 5%, about 10%, about 15%, less than about 20%, about 20%, about 25%, or less than about 25%, total calories from fat. A diet lacking in fat content (0%) is also to be considered “low fat” for purposes of the present disclosure.

In some aspects, the preparations comprise a food product comprising a pharmaceutically effective amount of a synthetic triterpenoid 2-cyano-3,12-dioxooleana-1,9-dien-28-oic acid (CDDO), CDDO-EA, CDDO-derivative, or combination thereof, and a carrier material or solution. For example, the carrier material may comprise a nutritional food material and/or other food supplement preparation for a subject (animal chow, diet meal replacement, etc.).

The CDDO compound to be included as an active ingredient in the preparations and compositions, may be defined as having a general structure of Formula I:

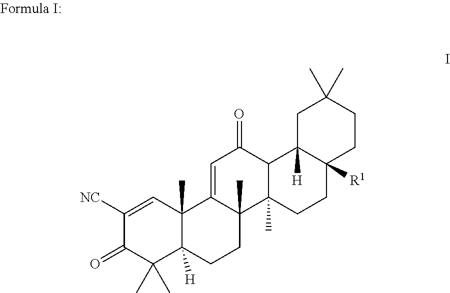

In certain embodiments, R¹ is a heteroatom-substituted or heteroatom-unsubstituted C1-C15 acyl.

In other embodiments, the CDDO compound included as a component of the present preparations and food products may comprise one or any combination of two or more of the following CDDO compounds:

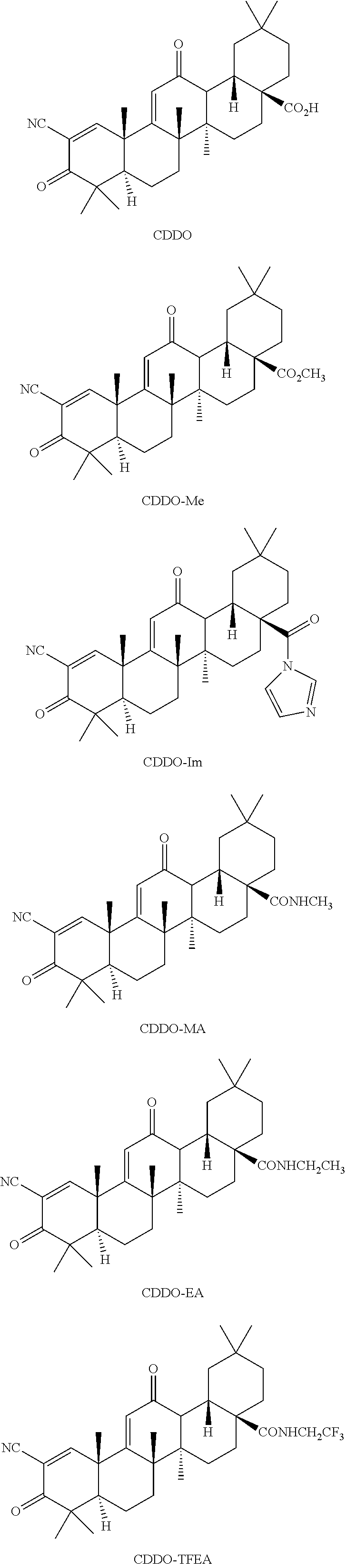

In another aspect of this invention, the CDDO compound as a component of the herein described methods and compositions may be described as having a structure as defined according to Formula II:

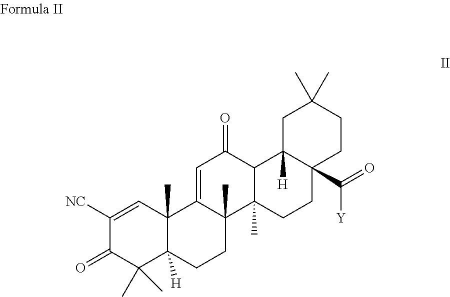

In some embodiments, the group Y is ethylamino or a heteroatom-substituted C₁-C₅-alkylamino having at least one fluorine atom. In other embodiments, the Y is a heteroatom-substituted or heteroatom-unsubstituted C₂-C₄-alkylamino having at least one fluorine atom. In further embodiments, the invention provides pharmaceutically acceptable salts and hydrates of these new synthetic triterpenoids. In yet further embodiments, the invention provides single enantiomers of these new synthetic triterpenoids or their salts or hydrates that are substantially free from other optical isomers. In still further embodiments, racemic mixtures of these new synthetic triterpenoids as well as their salts and hydrates are provided.

Examples of some of the CDDO compounds included in the present preparations and methods include one or any combination or one or more of CDDO, CDDO-Me, CDDO-EA, and CDDO-TFEA. The structures for CDDO-TFEA and CDDO-EA are presented below:

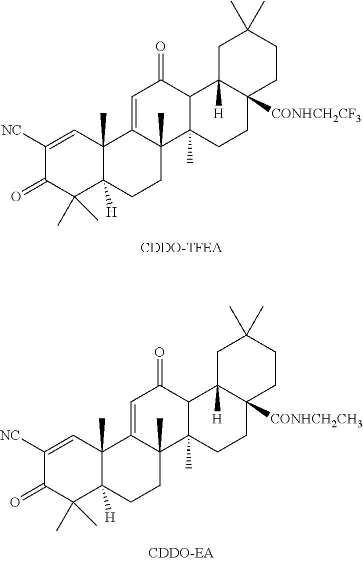

In other embodiments, R¹ is substituted to provide a CDDO-EA compound, having a structure of Formula III:

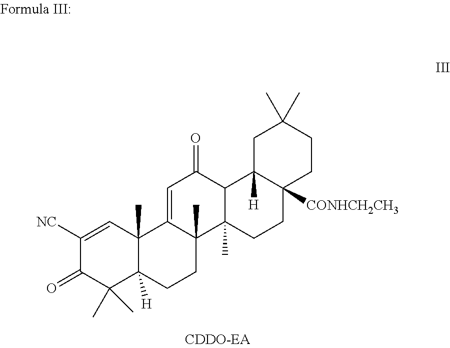

According to other aspects of the invention, orally consumable food and/or nutritional or dietary supplement products are provided that comprise one or more synthetic CDDO, CDDO analogs and/or CDDO-derivatives. The food product may comprise an orally ingestible food product suitable for veterinary or human consumption. For example, specialized veterinary food products such as chow mix, advanced animal diet care preparations, animal food products for elderly/advanced age animals or metabolically challenged animals, overweight animals, may be prepared. In one embodiment the veterinary food product is a chow mix, that includes about 200 mg to about 500 mg of the synthetic CDDO compound (such as CDDO-EA) in about 1 kilogram of the chow and/or consumable product. The chow mix, for example, may be a high fat (about 40%, 50%, 60%, or higher (70%, 80%, 90%) calories from fat) or a low fat (about 5%, 10%, 15%, 20% to about 20% calories from fat) chow diet.

High fat or low fat content products that comprise one or a combination of the CDDO or CDDO analog and/or derivative compounds, that are suitable for human consumption, are also provided.

Novel Improved Syntheses of CDDO: Improved Methods Avoid Over Reacted Bromination, avoid Complex Mixture of Product, Reduce Synthesis Time 14 fold (2 hours):

In another aspect, an improved, more efficient chemical method for synthesizing a synthetic triterpenoid is provided. The methods provided here are improved over those conventional in the literature. The method conventional in the literature provides for a conversion of Oleanolic acid to an intermediate product #6 (with a high catalyst (HBr) eq) (Fu et al. (2013)). The present process provides for use of a low catalytic amount of HBr. The prior art process in Fu et al. results in production of a complex mixture of compounds, and random, undesired multi-brominated substitutions and/or sites of substitution groups, as well as alpha bromination occurs. Subsequent elimination resulted in a tetrasubstituted olefine. The methyl ester was hydrolyzed, and the corresponding carboxylic acid resulted. These incidents renders the conventional, prior processes unacceptable.

The present method provides a synthesis method that employs LiI to transform the methyl ester of the CDDO-Me to a carboxylic acid (CDDO) from substrate. The CDDO resulting was not a complex mixture. Chemical conversion from Oleanolic acid to a final product of CDDO-EA may take place either through the use of one or a mixture of substrates, this being either a mixture of an epoxide CDDO compound (#4) or a ketone CDDO compound (#5). The CDDO Compound #6 obtained as a result of the bromination step (HBr, Br₂), may then be converted to CDDO-Me (Compound #7) in the synthesis step with CuCN, KI (DMF). The Compound #7 may then be processed (with LiI) to provide Compound #8 (CDDO). The Compound #8 is then subjected to (COCl)₂ to provide Compound #9. Compound #9 is then processed with EtNH₂ to provide Compound #10 (CDDO-EA).

A mixture of the epoxide and the ketone compound is brominated to provide a CDDO-Me of the desired structure, and this CDDO-Me product would then be processed to provide a CDDO #8 using lithium iodine (LiI) (See FIG. 19).

The bromination reaction was run with substrate of both epoxide (#4) and ketone (#5) compounds using a much reduced amount of the acid catalyst (acid catalyst, HBr) (less than about 1% weight equivalents per substrate weight). The weight equivalent of acid catalyst found effective in the present methods (HBr, 0.025 eq) were significantly less than the catalyst equivalent weight reported in the literature of Fu et al. (2013) (HBr, 0.44 eq). In addition, while the prior methods required at least 24 hours for chemical synthesis of bardoxolone methyl (CDDO-Me), the presently disclosed chemical synthesis pathway provides for a much shorter synthesis time of about 2 hours to synthesize CDDO-Me (Compound #7), compared to the literature reported time period of 24 hours (Fu et al. (2013)).

The significantly reduced equivalent amount of acid catalyst required in the present synthesis scheme, the greater degree of specificity of reaction products, and the vastly shorted processing time (2 hours), provides a commercially-scalable technique for the synthesis and commercial manufacture of CDDO compounds, including CDDO-Me, as well as further synthesis to CDDO and CDDO-EA. (See FIG. 19). This is also a commercially scalable manufacturing option for production of these very valuable chemical materials. These materials include, in particular, the synthesis of CDDO, CDDO-Me, and CDDO-EA products.

A flow-chart of the chemical synthesis for the CDDO appears in FIG. 19. The synthesis of CDDO-EA is also presented in Example 16 (a CDDO-EA Batch 1) and Example 17 (a CDDO-EA Batch #2). The CDDO-EA produced from each batch were of essentially the same purity and biological activity. The anti-inflammatory properties of both batches of CDDO-EA produced was assessed as a function of the effectiveness of the CDDO-EA for blocking LPS-induced MCP-1 production in cell culture (macrophages). It was found that the effectiveness of the Batch #1 and the Batch #2 CDDO-EA was essentially the same (LPS=112,235 μg/ml, Batch #1 CDDO-EA=31,341 μg/ml MCP-1 production, Batch #2 CDDO-EA=24,648 μg/ml MCP-1 production). This is an about 3.6 and a 4.6 fold difference in the decrease of MCP-1 production in cell culture, respectively, resulting from the Batch #1 CDDO-EA and the Batch #2 CDDO-EA. This establishes that the novel synthesis protocol developed in the present disclosure is reproducible and reliable.

FIGS. 17 and 18 provide the spectral analysis of the products obtained in the synthesis according to the present synthesis techniques, employing less than 5% of the acidic catalyst, and providing for production of the CDDO analog and/or derivative, such as CDDO-EA, in about a 2 hour period of time, at a temperature of 35° C.

Any embodiment discussed herein with respect to one aspect of the invention applies to other aspects of the invention as well, unless specifically noted otherwise.

Other objects, features and advantages of the present invention will become apparent from the following detailed description and any accompanying drawings. It should be understood, however, that the detailed description and any specific examples or drawings provided, while indicating specific embodiments of the invention, are given by way of illustration only, since various changes and modifications within the spirit and scope of the invention will become apparent to those skilled in the art from this detailed description.

### DESCRIPTION OF THE SEVERAL VIEWS OF THE DRAWINGS

FIGS. 1A-1B—CDDO-EA prevents obesity. FIG. 1A-Mice fed a high fat diet (HFD, 60% calories from fat) for 6 weeks evidenced significant weight gain (starting weight 24 grams, ending weight 38 grams) and were considered to be obese (greater than about 30% body fat, a BMI of at least about BMI 30); FIG. 1B-mice fed a HFD and CDDO-EA (400 mg/kg diet) for six weeks did not evidence a significant weight gain, and, were not considered obese. A synthesized batch of CDDO-EA was prepared according to the flow chart of FIG. 19. This CDDO-EA was combined into commercially available laboratory animal chow preparations (400 mg CDDO-EA+1,000 g High Fat animal chow; 400 mg CDDO-EA+1,000 g Low Fat animal chow).

FIG. 2—Novel synthesis protocol for synthesis of CDDO-EA. The CDDO-EA analog possesses modifications at C28. The arrows (1-5) indicate the synthesis steps that were modified to provide a reduced ratio of catalyst (HBr) equivalent per substrate equivalent, in providing for synthesis of the intermediate compound, #6 (See FIG. 20).

FIGS. 3A-3B—High purity and correct mass of synthesized CDDO-EA (FIG. 3A). HPLC chromatogram of synthesized CDDO-EA (FIG. 3B). The experimental APCI-MS spectrum of synthesized CDDO-EA and expected spectrum of known CDDO-EA.

FIG. 4—CDDO-EA prevents obesity. C57B16/J mice were fed a LFD (10% calories from fat) or HFD (60% calories from fat) with or without incorporation of CDDO-EA (400 mg/kg chow) in the chow for 6 weeks (n=5 or 7). Data are represented as mean±SEM (Two-Way Repeated Measures ANOVA). *p<0.05, ***p<0.001, ****p<0.0001). A synthesized batch of CDDO-EA was prepared according to the flow chart of FIG. 19. This CDDO-EA was combined into animal chow preparations (400 mg CDDO-EA+1,000 g High Fat animal chow; 400 mg CDDO-EA+1,000 g Low Fat animal chow).

FIG. 5—CDDO-EA inhibits energy intake. C57B16/J mice were fed a LFD (10% calories from fat) or HFD (60% calories from fat) with or without incorporation of CDDO-EA (400 mg/kg diet) in the chow for 6 weeks (n=3-7). Data are represented as mean±SEM (Two-Way ANOVA, Tukey's multiple comparisons test). ****p<0.001, HFD vs. HFD+CDDO-EA at 1 wk; **p=0.003 HFD vs. HFD+CDDO-EA at 3 wk; ****p<0.001, HFD vs. HFD+CDDO-EA at 4 wk; ***p=0.0001, HFD vs. HFD+CDDO-EA at 5 wk; ***p=0.002, HFD vs. HFD+CDDO-EA at 6 wk). HFD=triangle/solid; HFD+CDDO-EA=upside down triangle/solid. A synthesized batch of CDDO-EA was prepared according to the flow chart of FIG. 19. This CDDO-EA was combined into animal chow preparations (400 mg CDDO-EA+1,000 g High Fat animal chow; 400 mg CDDO-EA+1,000 g Low Fat animal chow).

FIGS. 6A-6B-CDDO-EA prevents glucose intolerance. Serum glucose (FIG. 6A) measurements obtained from oral glucose tolerance tests (OGTTs) performed in mice (n=5 to 7) before and after the experimental feeding of low fat diet (LFD, 10% calories from fat) and (FIG. 6B) high fat diet (HFD, 60% calories from fat) with or without CDDO-EA (400 mg/kg diet) in the chow for 6 weeks. *p<0.001 vs. HFD; One-Way ANOVA. The (FIG. 6A) left panel of each graph corresponds to LFD and the (FIG. 6B) right panel of each graph corresponds to HFD. The gray shaded area in FIG. 6A represents the mean value glucose concentration (basal levels) before experimental feeding. The gray shaded area in FIG. 6B represents the mean values of glucose (FIG. 6B) concentration (basal levels) before experimental feeding. The error bars correspond to 95% confidence intervals (without CDDO-EA= [symbol] ; with CDDO-EA= [symbol] .

A synthesized batch of CDDO-EA was prepared according to the flow chart of FIG. 19. This CDDO-EA was combined into animal chow preparations (400 mg CDDO-EA+1,000 g High Fat animal chow; 400 mg CDDO-EA+1,000 g Low Fat animal chow).

FIGS. 7A-7B-CDDO-EA prevents hyperinsulinemia. Serum insulin measurements obtained from OGTTs performed in mice (n=5 to 7) before and after the experimental feeding of low fat diet (LFD, 10% calories from fat) and high fat diet (HFD, 60% calories from fat) with or without CDDO-EA (400 mg/kg diet) in the chow for 6 weeks. *p<0.001 vs. HFD; One-Way ANOVA. FIG. 7A at the left panel corresponds to LFD and FIG. 7B at the right panel corresponds to HFD. The gray shaded area represents the mean values of insulin concentration (basal levels) before experimental feeding. The error bars correspond to 95% confidence intervals; without CDDO-EA= [symbol] ; with CDDO-EA= [symbol] . A synthesized batch of CDDO-EA was prepared according to the flow chart of FIG. 19. This CDDO-EA was combined into animal chow preparations (400 mg CDDO-EA+1,000 g High Fat animal chow; 400 mg CDDO-EA+1,000 g Low Fat animal chow).

FIGS. 8A-8B—CDDO-EA inhibits lipopolysaccharide (LPS)-induced NF-κB and Inhibitory kappa B alpha (IκB-α) phosphorylation. In FIG. 8A L6-GLUT4myc myotubes were pretreated with 500 nM CDDO-EA for 1 hr then exposed to 100 ng/ml LPS for 1 hr. Myotubes were harvested for analysis by Western blot using antibodies against phosphorylated NF-κB p65 (Ser536), total NF-κB, phosphorylated IκB-α (Ser32), IκB-α total protein, and actin. NF-κB and 1 κB-α phosphorylation was normalized to NF-κB and IκB-α total protein, respectively, then expressed as percent over control sample (mean±SEM). *p<0.05; ***p<0.001; (One-Way ANOVA). Four independent experiments were run in duplicate or triplicate. FIG. 8B. L6-GLUT4myc myotubes were treated with doubling doses of CDDO-EA for 6 or 24 hours. Cell viability was normalized to non-treated myotubes. Two independent experiments were run in triplicate.

FIG. 9—CDDO-EA blocks NF-κB transcriptional activity. L6-GLUT4myc myotubes were transfected with a NF-κB luciferase reporter plasmid then treated with 500 nM CDDO-EA for 1 hr followed by 100 ng/ml LPS for 6 hours. Data indicates luciferase values normalized to total protein concentration. Experiments were run in duplicate. *p<0.05; **p<0.01; (One-Way ANOVA); RLU, relative light unit.

FIGS. 10A-10B—CDDO-EA inhibits LPS-inducted TNF-α and MCP-1 gene expression. L6-GLUT4myc myotubes were exposed to 500 nM CDDO-EA for 1 hr followed by 1 hr exposure with 100 ng/ml LPS. mRNA expression levels of TNF-α and MCP-1 were measured by RT-PCR. Data is representative of two independent experiments run in triplicate (mean±SEM). **p<0.01; ***p<0.001; (One-Way ANOVA).

FIG. 11—CDDO-EA inhibits LPS-induced cytokines secretion in macrophages. RAW264.7 mouse macrophages were pre-treated with 500 nM CDDO-EA for 1 hour then exposed to 100 ng/ml LPS for 6 hours. TNF-α levels were measured and quantified by ELISA. Three independent experiments were run in duplicate or triplicate. Data represented as mean±SEM. *p<0.05 (One-Way ANOVA).

FIGS. 12A-12D—CDDO-EA blocks LPS induced IL-1β, IL-6, and MCP-1 secretion in THP-1 human macrophages. THP-1 macrophages were pre-treated with 500 nM CDDO-EA for 1 hour then exposed to 100 ng/ml LPS for 6 hours. Cytokine and chemokine levels were measured and quantified by Bio-Plex Pro-Human 17-plex ELISA kit. Experiment was run in triplicate. Data represented as mean±SEM. ** P<0.01; *** P<0.001 (One-Way ANOVA). NS; not significant. 12A=IL-1β; 12B=MCP-1; 12C=IL-6; 12D=TNF-α.

FIGS. 13A-13B—FIG. 13A—CDDO-EA prevents glucose intolerance. Serum glucose measurements obtained from OGTTs performed in mice (n=5 to 7) before and after experimental feeding of 400 mg/kg diet for 0 weeks, 2 weeks, 4 weeks and 6 weeks of: Low Fat diet (10% calories from fat); Low Fat diet+CDDO-EA (400 mg CDDO)-EA in 1 kilogram animal chow); High Fat diet (60% calories from fat); High Fat diet+CDDO-EA (400 mg CDDO-EA per 1 kilogram animal chow). (Two-Way ANOVA, Tukey's multiple comparisons test; **p<0.003, HFD vs. HFD+CDDO-EA at 2 wk; ****p<0.0001 HFD vs. HFD+CDDO-EA at 4 wk; **** p<0.0001, HFD vs. HFD+CDDO-EA at 6 wk). FIG. 13B—CDDO-EA prevents hyperinsulinemia. Serum insulin levels obtained from OGTTs performed in mice (n=5 to 7) before and after experimental feeding of 400 mg/kg diet for 0 weeks, 2 weeks, 4 weeks and 6 weeks of: Low Fat diet (10% calories from fat); Low Fat diet+CDDO-EA; High Fat diet (60% calories from fat) alone; High Fat diet+CDDO-EA. A synthesized batch of CDDO-EA was prepared according to the flow chart of FIG. 19. This CDDO-EA was combined into animal chow preparations (400 mg CDDO-EA+1 kilogram High Fat animal chow; 400 mg CDDO-EA+1 kg Low Fat animal chow). (Two-Way ANOVA, Tukey's multiple comparisons test; ** p<0.0001, HFD vs. HFD+CDDO-EA at 2 wk; *p=0.0029 HFD vs. HFD+CDDO-EA at 4 wk; **** p<0.0001, HFD vs. HFD+CDDO-EA at 6 wk).

FIGS. 14A-14B—CDDO affects NF-κB translocation to the cell nucleus. L6-GLUT4myc myotubes were pre-treated with 500 nM CDDO-EA for 1 hr followed by 100 ng/ml LPS for 1 hr. 14A) For immunofluorescence, fixed and permeabilized myotubes were stained using DAPI (nuclei, blue) and anti-NF-κB p65 primary antibody and a secondary antibody conjugated with Alexa Fluor® 488. 14A—NF-κB was presented with green fluorescence in cytoplasm and nuclei (red arrows). Scale bar: 50 μm. Images are representative of two independent experiments. 14B—Cytoplasmic and nuclear fractions were isolated and immunoblotted for p65 NF-κB and GAPDH and lamin to verify cytoplasmic and nuclear fractions, respectively. Data are representative of three independent experiments run in duplicate.

FIG. 15—CDDO-EA (synthesized) significantly suppresses the LPS-induced production of the pro-inflammatory chemokine, monocyte chemotactic protein-1 (MCP-1), in mouse macrophages similar to that observed with reference CDDO-EA (ORG CDDO). The synthesized CDDO-EA produced provided the same and/or similar biological activity in mouse macrophages compared to the reference CDDO-EA (ORG CDDO-EA).

FIGS. 16A-16B—CDDO-EA activates GLUT4 translocation to cell membrane and p38 phosphorylation. L6-GLUT4myc myotubes were pre-treated with 500 nM CDDO-EA for 1 hr followed by 100 ng/ml LPS for 1 hr. 100 nM insulin treatment was for 15 min. 16A) For immunofluorescence, myotubes were fixed without permeabilization and stained using DAPI (nuclei, blue) and anti-c-myc primary antibody (c-myc epitope-tagged GLUT4 distinguishes from endogenous GLUT4) and a secondary antibody conjugated with Alexa Fluor® 488. c-myc was presented with green fluorescence. Scale bar: 50 μm. 16B) Myotubes were harvested for analysis by Western blot using antibodies against phosphorylated p38, total p38, and actin. p38 phosphorylation was normalized to p38 total protein, then expressed as percent over control sample. Three independent experiments were run in triplicate. Data are represented as mean±SEM. *** P<0.001; (unpaired t test, two tailed).

FIG. 17—NMR-spectra analysis of combined preparations of compound #6 (the brominated compound, bardoxolone methyl (CDDO-Me) before purification. (one preparation using a reduced catalyst (HBr) amount (about 5% that provided in the literature) and the second preparation synthesized with an even lesser amount of catalyst). For comparison, the spectrum of the brominated product previously obtained was stacked. The spectra evidence that a preparation having a more homogenous compound #6 was obtained by using even lower amounts than 5% of that reported in the literature, of catalyst, resulted in superior preparations with less contaminants.

FIG. 18—NMR-NMR spectra analysis of a single preparation of compound #6 (the brominated compound, bardoxolone methyl (CDDO-Me) before purification, using less than 5% concentration of catalyst (HBr), compared to catalyst amounts reported in the literature for bromination. Spectra show evidence of some other peaks, and thus the presence of compounds of other than the desired #6 compound (properly brominated compound #6).

FIG. 19—Chemical Synthesis of CDDO-EA. Synthesis of CDDO-Me from a brominated compound (#6) was provided using a very low amount of catalyst (0.025 eq HBr). A unique ratio of catalyst and Br₂ (HBr (0.025 eq.+Br₂ (2.4 eq), was found to successfully provide the non-alpha brominated precursor compound #6. This compound #6 is then further processed to intermediate compound #7 (CDDO) via CuCN, KI/DMF). The intermediate product #7 (CDDO-Me) is then further processed (LiI) to provide compound #8 (CDDO). The compound #8, (CDDO) is then further processed ((COCl)₂) to synthesize the intermediate compound #9. The compound #9, may then be further processed (EtNH₂) to provide the compound #10 (CDDO-EA). For clarity, Compound #4 is methyl-protected CDDO with epoxide, and compound #5 is methyl-protected CDDO with ketone.

FIG. 20—Chemical synthesis of CDDO-EA according to Fu et al. (2013) was unsuccessful. Changes in the amount of catalyst were required in order to avoid production of an over-brominated compound #11, and to instead provide the properly brominated compound #6. This was accomplished by modifying the Fu et al protocol, specifically by changing the ratio of catalyst (HBr) (Fu et al., HBr (0.44 e.g.)+Br₂ (2.4 eq)). In the presently disclosed synthesis, the equivalent of catalyst HBr was significantly reduced (HBr (0.025 eq.)+Br₂ (2.4 eq)). According to the presently disclosed synthesis methods, a properly mono-brominated intermediate compound #6 could then be obtained, and then processed to provide compound #7 (CDDO-Me), as depicted in FIG. 20, and then compound #7 was further processed to synthesize the appropriate #8 compound (CDDO) using lithium iodide to transform the methyl ester to carboxylic acid. (See FIG. 20). The appropriate compound #8, (CDDO), was then further processed to synthesize the intermediate compound #9. From this, a compound #9 was provided, and was further processed to synthesize compound #10 (CDDO-EA).

FIG. 21—A first batch (CDDO-EA Batch #1) and a second batch (CDDO-EA Batch #2) were compared for ability to act as an anti-inflammatory agent. The second batch of CDDO-EA was tested for its biological property of anti-inflammatory activity. As with the first batch of CDDO-EA, this was tested by using the mouse macrophage cell line RAW264.7. RAW264.7 macrophages were pre-treated with 500 nM CDDO-EA for 1 hour then stimulated with 100 ng/ml of lipopolysaccharide (LPS) for 6 hr. Supernatants were collected, and an enzyme linked immunoassay (ELISA) was performed to detect the pro-inflammatory protein, monocyte chemotactic protein-1 (MCP-1). The macrophages were also treated with LPS alone or with CDDO-EA alone. A control (no treatment) group was also included in the study. The second batch CDDO-EA blocked the LPS-induced production of MCP-1 in RAW264.7 macrophages. This suppression of MCP-1 production by the second batch CDDO-EA is similar to that observed for the first batch of CDDO-EA. These findings show that the second batch of CDDO-EA has anti-inflammatory properties similar to the first batch of CDDO-EA, and that the synthesis procedure provides consistent, repeatable product that demonstrate predictable and consistent biological activity.

FIG. 22A and FIG. 22B. CDDO-EA does not affect energy expenditure. FIG. 22A. EE was measured 289 times in a 24-hour cycle at each day from 3-9 days of the experimental feeding. FIG. 22B. Average EE for days 3-9 of experimental feeding. C57BL/6J mice were fed a HFD with or without incorporation of CDDO-EA (diet containing 0.04% CDDO-EA) or LFD or HFD pair-fed for 6 weeks (n=5).

FIG. 23—CDDO-EA decreases food intake. C57BL/6J mice were fed a HFD with or without incorporation of CDDO-EA (diet containing 0.04% CDDO-EA) or LFD or HFD pair-fed for 6 weeks (n=5). Food intake data points in week 1 are not recorded when mice were in the Prometheion. Data are represented as mean±SEM (Two-way Repeated Measures ANOVA). *p<0.05; **p<0.01 vs. HFD pair-fed; [symbol] p<0.01 vs. HFD+CDDO-EA; ##p<0.01 vs. LFD ad lib.

FIG. 24—Body composition at 42 days. Body composition was measured by NMR. C57BL/6J mice were fed a HFD with or without incorporation of CDDO-EA (diet containing 0.04% CDDO-EA) or LFD or HFD pair-fed for 6 weeks (n=5). BW=body weight. Data are represented as mean±SEM (Two-way Repeated Measures ANOVA).

FIG. 25—OGTT Area Under the Curve. Oral GTT performed at week 6 of experimental feeding, and mice were dosed on lean mass (2 g/kg lean mass). C57BL/6J mice were fed a HFD with or without incorporation of CDDO-EA (diet containing 0.04% CDDO-EA) or LFD or HFD pair-fed for 6 weeks (n=5). Data are represented as mean±SEM (One-way ANOVA).

FIG. 26—CDDO-EA lowers fasting glucose levels. Fasting glucose levels were measured at day 42 of experimental feeding. C57BL/6J mice were fed a HFD with or without incorporation of CDDO-EA (diet containing 0.04% CDDO-EA) or a LFD or were HFD pair-fed for 6 weeks (n=5). Data are represented as mean±SEM (One-way ANOVA).

FIG. 27—siRNA of ERK1 and ERK2. RAW264.7 cells were transfected in duplicate with 500 ng of siRNAs targeting ERK1 or ERK2 for 48 hours, as per Lipofectamine 3000 Reagent protocol.

FIG. 28—CDDO-EA inhibits PA-induced NFκB phosphorylation. L6-GLUT4myc myotubes were pretreated with CDDO-EA (500 nM, 1 h) and then treated with PA (400 μM, 6 h). NFκB phosphorylation and protein were measured by Western blotting. Data are represented as ±SEM. * P<0.05 vs. control; ^{#}P<0.05 vs. LPS. (One-Way ANOVA).

FIG. 29—CDDO-EA blocks TNF-α and MCP-1 gene expression. L6-GLUT4myc myotubes were pretreated with CDDO-EA (500 nM, 1 h) and treated PA (400 μM, 6 h). TNF-α and MCP-1 gene expression were measured by RT-PCR. Data are represented as +SEM *P<0.05 vs. control, ^{#}P<0.05 vs. control. * P<0.05 vs. LPS, $P<0.05 vs. PA. (One-Way ANOVA).

### DETAILED DESCRIPTION OF THE INVENTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

### Definitions

Any term of degree such as, but not limited to, the term “about” as used in the description and the appended claims, should be understood to include the recited values or a value that is three times greater or one third of the recited values. For example, about 3 cm includes all values from 1 mm to 9 cm. For example, terms of degree can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The term “disease” is intended to be interpreted as any condition of a subject evidencing symptoms associated with a clinical determination of obesity, a medical determination of an overweight measurement, metabolic syndrome, cardiac disease, insulin insensitivity, hypoglycemia, type I or II diabetes, elevated blood pressure, glucose intolerance, and the like. In particular descriptions of the preparations and methods, the term “disease” may be intended to specifically indicate a subject having been determined to be clinically obese or overweight.

The terms “comprising,” “including” and “having” are used interchangeably in this disclosure. The terms “comprising,” “including” and “having” mean to include, but not necessarily be limited to the things so described.

The term, “synthetic”, as provided in a description of a chemical or compound, should be understood to mean a non-naturally occurring substance or compound, such as a substance of a compound that has been obtained from other than a naturally occurring cell or tissue in nature. The term “synthetic” may also refer to a substance of compound that has been chemically modified from the chemical structure of the substance or compound as it exists in its native unchanged state in nature.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the term “amino” means —NH₂; the term “nitro” means —NO₂; the term “halo” designates —F, —Cl, —Br or —I; the term “mercapto” means —SH; the term “cyano” means —CN; the term “silyl” means —SiH₃, and the term “hydroxy” means-OH.

The term “heteroatom-substituted,” when used to modify a class of organic radicals (e.g., alkyl, aryl, acyl, etc.), means that one, or more than one, hydrogen atom of that radical has been replaced by a heteroatom, or a heteroatom containing group. Examples of heteroatoms and heteroatom containing groups include: hydroxy, cyano, alkoxy, —O, ═S, —NO₂, —N(CH₃)₂, amino, or —SH. Specific heteroatom-substituted organic radicals are defined more fully below.

The term “heteroatom-unsubstituted,” when used to modify a class of organic radicals (e.g., alkyl, aryl, acyl, etc.) means that none of the hydrogen atoms of that radical have been replaced with a heteroatom or a heteroatom containing group. Substitution of a hydrogen atom with a carbon atom, or a group consisting of only carbon and hydrogen atoms, is not sufficient to make a group heteroatom-substituted. For example, the group —C₆H₄C≡CH is an example of a heteroatom-unsubstituted aryl group, while —C₆H₄F is an example of a heteroatom-substituted aryl group. Specific heteroatom-unsubstituted organic radicals are defined more fully below.

The term “alkyl” includes straight-chain alkyl groups, branched-chain alkyl groups, cycloalkyl (alicyclic) groups, alkyl heteroatom-substituted cycloalkyl groups, and cycloalkyl heteroatom-substituted alkyl groups. The term “heteroatom-unsubstituted Cₙ-alkyl” refers to a radical having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃, —CH₂CH₃, —CH₂CH₂CH₃, —CH(CH₃)₂, —CH(CH₂)₂ (cyclopropyl), —CH₂CH₂CH₂CH₃, —CH(CH₃)CH₂CH₃, —CH₂CH(CH₃)₂, —C(CH₃)₃, —CH₂C(CH₃)₃, cyclobutyl, cyclopentyl, and cyclohexyl, are all examples of heteroatom-unsubstituted alkyl groups. The term “heteroatom-substituted Cₙ-alkyl” refers to a radical having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all examples of heteroatom-substituted alkyl groups: trifluoromethyl, —CH₂F, —CH₂Cl, —CH₂Br, —CH₂OH, —CH₂OCH₃, —CH₂OCH₂CH₃, —CH₂OCH₂CH₂CH₃, —CH₂OCH(CH₃)₂, —CH₂OCH(CH₂)₂, —CH₂OCH₂CF₃, —CH₂OCOCH₃, —CH₂NH₂, —CH₂NHCH₃, —CH₂N(CH₃)₂, —CH₂NHCH₂CH₃, —CH₂N(CH₃)CH₂CH₃, —CH₂NHCH₂CH₂CH₃, —CH₂NHCH(CH₃)₂, CH₂NHCH(CH₂)₂, —CH₂N(CH₂CH₃)₂, —CH₂CH₂F, —CH₂CH₂Cl, —CH₂CH₂Br, —CH₂CH₂I, —CH₂CH₂OH, —CH₂CH₂OCOCH₃, —CH₂CH₂NH₂, —CH₂CH₂N(CH₃)₂, —CH₂CH₂NHCH₂CH₃, —CH₂CH₂N(CH₃)CH₂CH₃, CH₂CH₂NHCH₂CH₂CH₃, CH₂CH₂NHCH(CH₃)₂, CH₂CH₂NHCH(CH₂)₂, CH₂CH₂N(CH₂CH₃)₂, —CH₂CH₂NHCO₂C(CH₃)₃, and —CH₂Si(CH₃)₃.

The term “heteroatom-unsubstituted Cₙ-alkenyl” refers to a radical having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂, —CH═CHCH₃, —CH═CHCH₂CH₃, —CH═CHCH₂CH₂CH₃, —CH═CHCH(CH₃)₂, —CH═CHCH(CH₂)₂, —CH₂CH═CH₂, —CH₂CH═CHCH₃, —CH₂CH═CHCH₂CH₃, —CH₂CH═CHCH₂CH₂CH₃, —CH₂CH═CHCH(CH₃)₂, —CH₂CH—CHCH(CH₂)₂, and —CH═CH—C₆H₅. The term “heteroatom-substituted Cₙ-alkenyl” refers to a radical having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, —CH═CHF, —CH═CHCl and —CH═CHBr, are examples of heteroatom-substituted alkenyl groups.

The term “heteroatom-unsubstituted Cₙ-alkynyl” refers to a radical having a linear or branched, cyclic or acyclic structure, further having at least one carbon-carbon triple bond, a total of n carbon atoms, at least one hydrogen atom, and no heteroatoms. For example, a heteroatom-unsubstituted C₂-C₁₀-alkynyl has 2 to 10 carbon atoms. The groups, —C≡CH, —C≡CCH₃, and —C≡CC₆H₅ are examples of heteroatom-unsubstituted alkynyl groups. The term “heteroatom-substituted Cₙ-alkynyl” refers to a radical having a single nonaromatic carbon atom as the point of attachment and at least one carbon-carbon triple bond, further having a linear or branched, cyclic or acyclic structure, and having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkynyl has 2 to 10 carbon atoms. The group, —C≡CSi(CH₃)₃, is an example of a heteroatom-substituted alkynyl group.

The term “heteroatom-unsubstituted Cₙ-aryl” refers to a radical having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Examples of heteroatom-unsubstituted aryl groups include phenyl, methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, C₆H₃(CH₃)CH₂CH₃, —C₆H₄CH—CH₂, —C₆H₄CH═CHCH₃, —C₆H₄C═CH, —C₆H₄C≡CCH₃, naphthyl, and the radical derived from biphenyl. The term “heteroatom-unsubstituted aryl” includes carbocyclic aryl groups, biaryl groups, and radicals derived from polycyclic fused hydrocarbons (PAHs). The term “heteroatom-substituted Cₙ-aryl” refers to a radical having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. The term “heteroatom-substituted aryl” includes heteroaryl groups. It also includes those groups derived from the compounds: pyrrole, furan, thiophene, imidazole, oxazole, isoxazole, thiazole, isothiazole, triazole, pyrazole, pyridine, pyrazine, pyridazine, pyrimidine, and the like. Further examples of heteroatom-substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OCOCH₃, —C₆H₄₀C₆H₅, —C₆H₄NH₂, —C₆H₄NHCH₃, —C₆H₄NHCH₂CH₃, —C₆H₄CH₂Cl, —C₆H₄CH₂Br, —C₆H₄CH₂OH, —C₆H₄CH₂OCOCH₃, —C₆H₄CH₂NH₂, —C₆H₄N(CH₃)₂, —C₆H₄CH₂CH₂Cl, —C₆H₄CH₂CH₂OH, —C₆H₄CH₂CH₂OCOCH₃, —C₆H₄CH₂CH₂NH₂, —C₆H₄CH₂CH═CH₂, —C₆H₄CF₃, —C₆H₄CN, —C₆H₄C═CSi(CH₃)₃, —C₆H₄COH, —C₆H₄COCH₃, —C₆H₄COCH₂CH₃, —C₆H₄COCH₂CF₃, —C₆H₄COC₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, imidazoyl, quinolyl and indolyl.

The term “heteroatom-unsubstituted Cₙ-aralkyl” refers to a radical having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C₇-C₁₀-aralkyl has 7 to 10 carbon atoms. Examples of heteroatom-unsubstituted aralkyls include phenylmethyl (benzyl) and phenylethyl. The term “heteroatom-substituted Cₙ-aralkyl” refers to a radical having a single saturated carbon atom as the point of attachment, further having a total of n carbon atoms, 0, 1, or more than one hydrogen atom, and at least one heteroatom, wherein at least one of the carbon atoms is incorporated in an aromatic ring structure, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms.

The term “heteroatom-unsubstituted Cₙ-acyl” refers to a radical having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to 10 carbon atoms. The groups, —COH, —COCH₃, —COCH₂CH₃, —COCH₂CH₂CH₃, —COCH(CH₃)₂, —COCH(CH₂)₂, —COC₆H₅, —COC₆H₄CH₃, COC₆H₄CH₂CH₃, —COC₆H₄CH₂CH₂CH₃, —COC₆H₄CH(CH₃)₂, —COC₆H₄CH(CH₂)₂, and —COC₆H₃ (CH₃)₂, are examples of heteroatom-unsubstituted acyl groups. The term “heteroatom-substituted Cₙ-acyl” refers to a radical having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The term heteroatom-substituted acyl includes carbamoyl, thiocarboxylate, and thiocarboxylic acid groups. The groups, —COCH₂CF₃, —CO₂H, —CO₂CH₃, —CO₂CH₂CH₃, —CO₂CH₂CH₂CH₃, —CO₂CH(CH₃)₂, —CO₂CH(CH₂)₂, —CONH₂, —CONHCH₃, —CONHCH₂CH₃, —CONHCH₂CH₂CH₃, CONHCH(CH₃)₂, —CONHCH(CH₂)₂, —CON(CH₃)₂, CON(CH₂CH₃)CH₃, CON(CH₂CH₃)₂ and —CONHCH₂CF₃, are examples of heteroatom-substituted acyl groups.

The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group.

The term “heteroatom-unsubstituted Cₙ-alkenyloxy” refers to a group, having the structure —OR, in which R is a heteroatom-unsubstituted Cₙ-alkenyl, as that term is defined above. The term “heteroatom-substituted Cₙ-alkenyloxy” refers to a group, having the structure —OR, in which R is a heteroatom-substituted Cₙ-alkenyl, as that term is defined above.

The term “heteroatom-unsubstituted Cₙ-alkynyloxy” refers to a group, having the structure —OR, in which R is a heteroatom-unsubstituted Cₙ-alkynyl, as that term is defined above. The term “heteroatom-substituted Cₙ-alkynyloxy” refers to a group, having the structure —OR, in which R is a heteroatom-substituted Cₙ-alkynyl, as that term is defined above.

The term “heteroatom-unsubstituted Cₙ-aryloxy” refers to a group, having the structure —OAr, in which Ar is a heteroatom-unsubstituted Cₙ-aryl, as that term is defined above. An example of a heteroatom-unsubstituted aryloxy group is —OC₆H₅. The term “heteroatom-substituted Cₙ-aryloxy” refers to a group, having the structure —OAr, in which Ar is a heteroatom-substituted Cₙ-aryl, as that term is defined above.

The term “heteroatom-unsubstituted Cₙ-aralkyloxy” refers to a group, having the structure —OR_{Ar}, in which R_{Ar} is a heteroatom-unsubstituted Cₙ-aralkyl, as that term is defined above. The term “heteroatom-substituted Cₙ-aralkyloxy” refers to a group, having the structure —OR_{Ar}, in which R_{Ar} is a heteroatom-substituted Cₙ-aralkyl, as that term is defined above.

The term “heteroatom-unsubstituted Cₙ-acyloxy” refers to a group, having the structure —OAc, in which Ac is a heteroatom-unsubstituted Cₙ-acyl, as that term is defined above. A heteroatom-unsubstituted acyloxy group includes alkylcarbonyloxy and arylcarbonyloxy groups. For example, —OCOCH₃ is an example of a heteroatom-unsubstituted acyloxy group. The term “heteroatom-substituted Cₙ-acyloxy” refers to a group, having the structure —OAc, in which Ac is a heteroatom-substituted Cₙ-acyl, as that term is defined above. A heteroatom-substituted acyloxy group includes alkoxycarbonyloxy, aryloxycarbonyloxy, carboxylate, alkylcarbonyl, alkoxycarbonyl, aminocarbonyl, and alkylthiocarbonyl groups.

The term “heteroatom-unsubstituted Cₙ-alkylamino” refers to a radical having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cₙ-alkylamino” includes groups, having the structure —NHR, in which R is a heteroatom-unsubstituted Cₙ-alkyl, as that term is defined above. A heteroatom-unsubstituted alkylamino group would include-NHCH₃, —NHCH₂CH₃, —NHCH₂CH₂CH₃, —NHCH(CH₃)₂, —NHCH(CH₂)₂, NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cₙ-alkylamino” refers to a radical having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cₙ-alkylamino” includes groups, having the structure —NHR, in which R is a heteroatom-substituted Cₙ-alkyl, as that term is defined above.

The term “heteroatom-unsubstituted Cₙ-alkenylamino” refers to a radical having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cₙ-alkenylamino” includes groups, having the structure —NHR, in which R is a heteroatom-unsubstituted Cₙ-alkenyl, as that term is defined above. Examples of heteroatom-unsubstituted Cₙ-alkenylamino groups also include dialkenylamino and alkyl (alkenyl)amino groups. The term “heteroatom-substituted Cₙ-alkenylamino” refers to a radical having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cₙ-alkenylamino” includes groups, having the structure —NHR, in which R is a heteroatom-substituted Cₙ-alkenyl, as that term is defined above.

The term “heteroatom-unsubstituted Cₙ-alkynylamino” refers to a radical having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cₙ-alkynylamino” includes groups, having the structure —NHR, in which R is a heteroatom-unsubstituted Cₙ-alkynyl, as that term is defined above. An alkynylamino group includes dialkynylamino and alkyl (alkynyl)amino groups. The term “heteroatom-substituted Cₙ-alkynylamino” refers to a radical having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cₙ-alkynylamino” includes groups, having the structure —NHR, in which R is a heteroatom-substituted Cₙ-alkynyl, as that term is defined above.

The term “heteroatom-unsubstituted Cₙ-arylamino” refers to a radical having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cₙ-arylamino” includes groups, having the structure —NHR, in which R is a heteroatom-unsubstituted Cₙ-aryl, as that term is defined above. A heteroatom-unsubstituted arylamino group includes diarylamino and alkyl (aryl)amino groups. The term “heteroatom-substituted Cₙ-arylamino” refers to a radical having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C₆-C₁₀-arylamino has 6 to 10 carbon atoms. The term “heteroatom-substituted Cₙ-arylamino” includes groups, having the structure —NHR, in which R is a heteroatom-substituted Cₙ-aryl, as that term is defined above. A heteroatom-substituted arylamino group includes heteroarylamino groups.

The term “heteroatom-unsubstituted Cₙ-aralkylamino” refers to a radical having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cₙ-aralkylamino” includes groups, having the structure —NHR, in which R is a heteroatom-unsubstituted Cₙ-aralkyl, as that term is defined above. An aralkylamino group includes diaralkylamino groups. The term “heteroatom-substituted Cₙ-aralkylamino” refers to a radical having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cₙ-aralkylamino” includes groups, having the structure —NHR, in which R is a heteroatom-substituted Cₙ-aralkyl, as that term is defined above. The term “heteroatom-substituted aralkylamino” includes the term “heteroaralkylamino.”

The term amido includes N-alkyl-amido, N-aryl-amido, N-aralkyl-amido, acylamino, alkylcarbonylamino, arylcarbonylamino, and ureido groups. The group, —NHCOCH₃, is an example of a heteroatom-unsubstituted amido group. The term “heteroatom-unsubstituted Cₙ-amido” refers to a radical having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cₙ-amido” includes groups, having the structure —NHR, in which R is a heteroatom-unsubstituted Cₙ-acyl, as that term is defined above. The term “heteroatom-substituted Cₙ-amido” refers to a radical having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group and the nitrogen atom at the point of attachment, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cₙ-amido” includes groups, having the structure —NHR, in which R is a heteroatom-unsubstituted Cₙ-acyl, as that term is defined above. The group, —NHCO₂CH₃, is an example of a heteroatom-substituted amido group.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Similarly, it is contemplated that one or more oxygen atom(s) of a compound of the present invention may be replaced by a sulfur or a selenium atom(s).

Any undefined valency on an atom of a structure shown in this application implicitly represents a hydrogen atom bonded to the atom.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylicacids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, and Use (P. H. Stahl & C. G. Wermuth eds., Verlag Helvetica Chimica Acta, 2002).

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are minor images of each other, like left and right hands. “Diastereomers” are stereoisomers of a given compound that are not enantiomers.

“Therapeutically effective amount” or “pharmaceutically effective amount” means that amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, the term “water soluble” means that the compound dissolves in water at least to the extent of 0.010 mole/liter or is classified as soluble according to literature precedence.

Other abbreviations used herein are as follows: DMSO, dimethyl sulfoxide; NO, nitric oxide; iNOS, inducible nitric oxide synthase; COX-2, cyclooxygenase-2; NGF, nerve growth factor; IBMX, isobutylmethylxanthine; FBS, fetal bovine serum; GPDH, glycerol 3-phosphate dehydrogenase; RXR, retinoid X receptor; TGF-β, transforming growth factor-β; IFNγ or IFN-γ, interferon-γ; LPS, bacterial endotoxic lipopolysaccharide; TNFα or TNF-α, tumor necrosis factor-α; IL-1β, interleukin-1β; MCP-1, monocyte chemotactic protein-1; GAPDH, glyceraldehyde-3-phosphate dehydrogenase; MTBE, methyl-tert-butylether; MTT, 3-[4,5-dimethylthiazol-2-yl]-2,5-diphenyltetrazolium bromide; TCA, trichloroacetic acid; HO-1, inducible heme oxygenase.

The above definitions supersede any conflicting definition in any of the reference that is incorporated by reference herein.

### Pharmaceutical Formulations and Routes of Administration

Administration of the compounds of the present invention to a subject will follow general protocols for the administration of pharmaceuticals, taking into account the toxicity, if any, of the active ingredient. It is expected that the treatment cycles would be repeated as necessary.

The compounds of the present invention may be administered by a variety of methods, especially orally. Depending on the route of administration, the active compounds may be coated by a material to protect the compound from the action of acids and other natural conditions which may inactivate the compound. Specific examples of formulations, including a polymer-based dispersion of CDDO-Me that showed improved oral bioavailability, are provided in U.S. Pat. No. 8,088,824, incorporated herein in its entirety. It will be recognized by those skilled in the art that other methods of manufacture may be used to produce dispersions of the present invention with equivalent properties and utility (see Repka et al., 2002 and references cited therein). Such alternative methods include but are not limited to solvent evaporation, extrusion, such as hot melt extrusion, and other techniques.

To administer the therapeutic compound, it may be necessary to coat the compound with, or co-administer the compound with, a material to prevent its inactivation. For example, the active ingredient compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984).

The therapeutic compound may also be administered parenterally. Dispersions may be prepared in, e.g., glycerol, liquid polyethylene glycols, mixtures thereof, and in oils. Under ordinary conditions of storage and use, these preparations may contain a preservative to prevent the growth of microorganisms.

Pharmaceutical compositions suitable for ingestion may include sterile aqueous solutions (where water soluble) or dispersions and sterile powders for the extemporaneous preparation of sterile solutions or dispersion. The composition may be sterile and may be fluid to the extent that easy administration routes are accommodated for the subject. The products must be stable under the conditions of manufacture and storage and must be preserved against the contaminating action of microorganisms such as bacteria and fungi.

The carrier may be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Generally, dispersions are prepared by incorporating the therapeutic compound into a sterile carrier which contains a basic dispersion medium and the required other ingredients from those enumerated above. In the case of sterile powders for the preparation of parenterally administrable solutions, the preferred methods of preparation are vacuum drying and freeze-drying which yields a powder of the active ingredient (i.e., the therapeutic compound) plus any additional desired ingredient from a previously sterile-filtered solution thereof.

The active ingredient may be orally administered, for example, with an inert diluent or an assimilable edible carrier (e.g., a food product). The active ingredient and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the active ingredient in the compositions and preparations may, of course, be varied. The amount of the active ingredient in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a subject.

The therapeutic compound may also be administered topically to the skin, eye, or mucosa. Alternatively, if local delivery to the lungs is desired the therapeutic compound may be administered by inhalation in a dry-powder or aerosol formulation.

The actual dosage amount of a compound of the present invention or composition comprising a compound of the present invention administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

Human Preparations/Food Preparations: In some embodiments, and in an anticipated food or other product for an average human of about 150 pounds, the pharmaceutically effective amount of the CDDO and/or CDDO analog or derivative, or combination thereof, is a daily dose from about 3 g to about 30 g of the compound. In some variations, the daily dose is from about 5 g to about 10 g of the compound. In some variations, the daily dose is from about 5 g to about 15 g of the compound. In some variations, the daily dose is about 8 to 10 g to about 20 g of the compound. In other variations, the daily dose is about 4 g to about 10 g of the compound. In still other variations, the daily dose is about 8 g of the active ingredient (CDDO-EA, etc.). In further variations, the daily dose is from about 5 mg to about 30 mg of the compound. In some variations, the daily dose is from about 5 mg to about 20 mg of the compound. In some variations, the daily dose is from about 10 g to about 30 g of the compound. In some variations, the daily dose is from about 10 g to about 100 g of the compound. In some variations, the daily dose is from about 10 g to about 50 mg of the compound.

In some embodiments, the pharmaceutically effective amount is a daily dose is about 5 g of compound for an adult human having a weight of about 150 pounds. In some variations, the daily dose is 5 to 20 g of compound. In some variations, the daily dose is about 1 to about 10 g of compound in an adult human having a weight of about 150 pounds. In some variations, the daily dose is 10 g of compound. In some variations, the daily dose is about 15 g of compound in an adult huma weighing about 150 pounds.

Veterinary Preparations/Animal Feed Formulation: For a veterinary animal, such as a dog or cat, a pharmaceutically effective amount is a daily dose is about 0.1-1000 g of compound per kg of body weight. In some variations, the daily dose is 0.15-20 g of compound per kg of body weight. In some variations, the daily dose is 0.20-10 g of compound per kg of body weight. In some variations, the daily dose is 0.40-30 g of compound per kg of body weight. In some variations, the daily dose is 0.50-90 g of compound per kg of body weight. In some variations, the daily dose is 0.60-80 g of compound per kg of body weight. In some variations, the daily dose is 0.70-70 g of compound per kg of body weight. In some variations, the daily dose is 0.80-6 mg of compound per kg of body weight. In some variations, the daily dose is 0.90-100 g of compound per kg of body weight. In some variations, the daily dose is from about 10 g to about 100 g of compound per day for a veterinary animal having a weight of about 15 pounds to about 60 pounds.

An effective amount typically will vary from about 0.001 mg/kg to about 1,000 mg/kg, from about 0.01 mg/kg to about 750 mg/kg, from about 0.1 mg/kg to about 500 mg/kg, from about 0.2 mg/kg to about 250 mg/kg, from about 0.3 mg/kg to about 150 mg/kg, from about 0.3 mg/kg to about 100 mg/kg, from about 0.4 mg/kg to about 75 mg/kg, from about 0.5 mg/kg to about 50 mg/kg, from about 0.6 mg/kg to about 30 mg/kg, from about 0.7 mg/kg to about 25 mg/kg, from about 0.8 mg/kg to about 15 mg/kg, from about 0.9 mg/kg to about 10 mg/kg, from about 1 mg/kg to about 5 mg/kg, from about 100 mg/kg to about 500 mg/kg, from about 1.0 mg/kg to about 250 mg/kg, or from about 10.0 mg/kg to about 150 mg/kg, in one or more dose administrations daily, for one or several days (depending, of course, of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some particular embodiments, the amount is less than 10,000 mg per day with a range, for example, of 750 mg to 9,000 mg per day.

The effective amount of the active ingredient (e.g., CDDO-EA, CDDO-Me, CDDO) may be less than 1 mg/kg/day, less than 500 mg/kg/day, less than 250 mg/kg/day, less than 100 mg/kg/day, less than 50 mg/kg/day, less than 25 mg/kg/day, less than 10 mg/kg/day, or less than 5 mg/kg/day. It may alternatively be in the range of 1 mg/kg/day to 200 mg/kg/day. For example, the unit dosage may be an amount that reduces blood glucose by at least 40%, and is comparable to that of a non-diabetic subject. In another embodiment, the unit dosage is an amount that reduces blood glucose to a level that is within +10% of the blood glucose level of a non-diabetic (Type 1 or Type 2) subject or a non-pre-diabetic subject.

In other non-limiting examples, a dose may also comprise from about 1 milligram/kg/body weight, about 5 milligrams/kg/body weight, about 10 milligrams/kg/body weight, about 50 milligrams/kg/body weight, about 100 milligram/kg/body weight, about 200 milligrams/kg/body weight, about 350 milligrams/kg/body weight, about 500 milligrams/kg/body weight, about 1 milligrams/kg/body weight, about 5 milligrams/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 1 mg/kg/body weight to about 5 mg/kg/body weight, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present invention may comprise, for example, at least about 0.1% of a compound of the present invention. In other embodiments, the compound of the present invention may comprise between about 2% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the subject has eaten or will eat.

Non-limiting specific formulations include CDDO-Me and/or CDDO-EA or other CDDO derivative/analog, in polymer dispersions (See U.S. Pat. No. 8,088,824), incorporated herein by reference. Some of the formulations reported therein exhibited higher bioavailability than either the micronized Form A or nanocrystalline Form A formulations. Additionally, the polymer dispersion based formulations demonstrated further surprising improvements in oral bioavailability relative to the micronized Form B formulations. For example, the methacrylic acid copolymer, Type C and HPMC-P formulations showed the greatest bioavailability in the subject monkeys.

### Combination Therapy

In addition to being used as a monotherapy, the compounds of the present invention may also find use in combination therapies. Effective combination therapy may be achieved with a single composition or pharmacological formulation that includes both agents, or with two distinct compositions or formulations, administered at the same time, wherein one composition includes a compound of this invention (CDDO, CDDO-Me, CDDO-EA, or other CDDO analog or derivative, or combination thereof), and the other includes the second agent(s). Alternatively, the therapy may precede or follow the other agent treatment by intervals ranging from minutes to months.

Various combinations may be employed, such as when a compound of the present invention is “A” and “B” represents a secondary agent, non-limiting examples of which are described below:

| | | | | | | |
|---|---|---|---|---|---|---|
| | A/B/A | B/A/B B/B/A | A/A/B | A/B/B B/A/A A/B/B/B | B/A/B/B | B/B/B/A |
| B/B/A/B | A/A/B/B | A/B/A/B | A/B/B/A | B/B/A/A B/A/B/A | B/A/A/B | A/A/A/B |
| B/A/A/A | A/B/A/A | A/A/B/A | | | | |

It is contemplated that other anti-inflammatory agents may be used in conjunction with the treatments of the current invention. For example, other COX inhibitors may be used, including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam. See also U.S. Pat. No. 6,025,395, which is incorporated herein by reference.

Dietary and nutritional supplements with reported benefits for treatment or prevention of Parkinson's, Alzheimer's, multiple sclerosis, amyotrophic lateral sclerosis, rheumatoid arthritis, inflammatory bowel disease, and all other diseases whose pathogenesis is believed to involve excessive production of either nitric oxide (NO) or prostaglandins, such as acetyl-L-carnitine, octacosanol, evening primrose oil, vitamin B6, tyrosine, phenylalanine, vitamin C, L-dopa, or a combination of several antioxidants may be used in conjunction with the compounds of the current invention.

Other particular secondary therapies include immunosuppressants (for transplants and autoimmune-related RKD), anti-hypertensive drugs (for high blood pressure-related RKD, e.g., angiotensin-converting enzyme inhibitors and angiotensin receptor blockers), insulin (for diabetic RKD), lipid/cholesterol-lowering agents (e.g., HMG-COA reductase inhibitors such as atorvastatin or simvastatin), treatments for hyperphosphatemia or hyperparathyroidism associated with CKD (e.g., sevelamer acetate, cinacalcet), dialysis, and dietary restrictions (e.g., protein, salt, fluid, potassium, phosphorus).

It should also be understood that, unless clearly indicated to the contrary, in any methods claimed herein that include more than one step or act, the order of the steps or acts of the method is not necessarily limited to the order in which the steps or acts of the method are recited.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1—Improved Synthesis of CDDO-EA

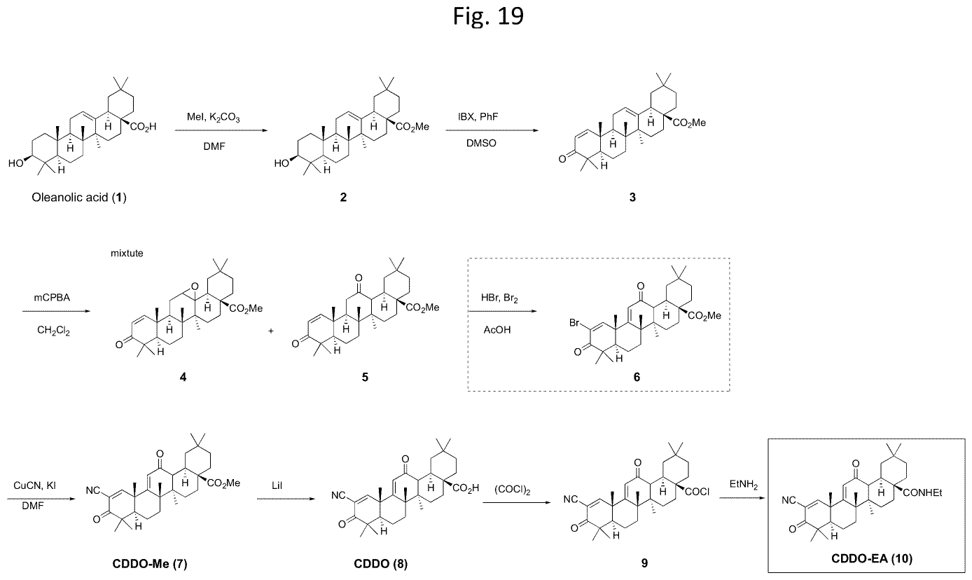

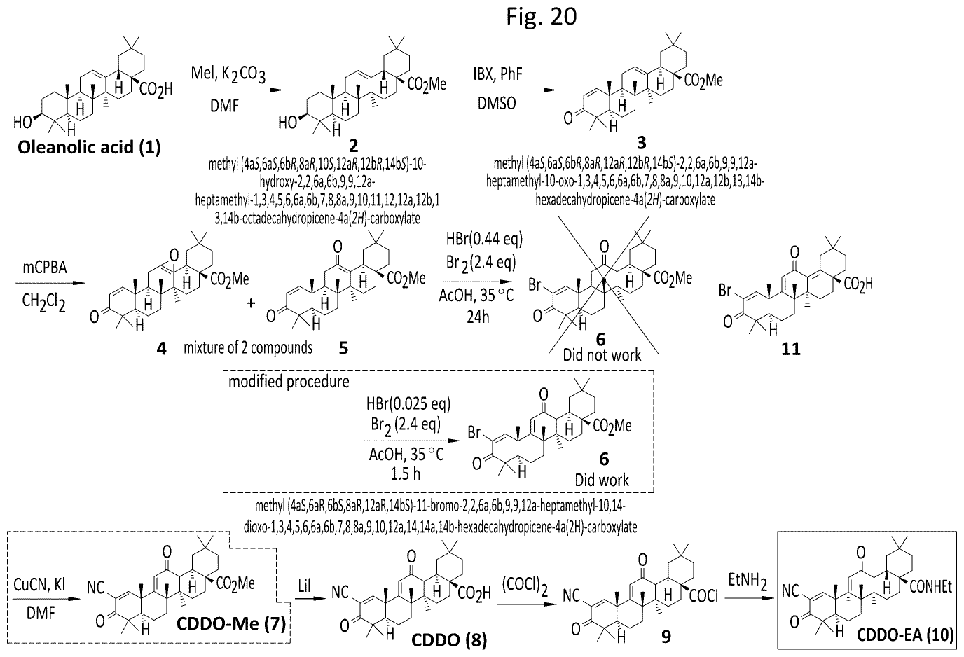

The synthesis methods for CDDO-Me (Compound #7) provided here, especially for the appropriately substituted brominated Compound #6 (see FIG. 19) are improved over those conventional in the literature. The methods conventional in the literature provide for a conversion of Oleanolic acid to CDDO-Me using lithium iodide (Fu et al. (2013). This process resulted in production of a complex mixture of compounds without specificity of the number of substitutions or site of substitution groups (See FIG. 20). Lithium iodide was used in the reaction of the present synthesis to transform the methyl ester of the CDDO-Me to a carboxylic acid (CDDO). The resulting CDDO product from this reaction was not a complex mixture. The CDDO obtained was then converted to CDDO-EA. (FIG. 19).

Chemical conversion from Oleanolic acid to a final product of CDDO-EA may take place either through the use of one or a mixture of substrates (an epoxide (Compound #4), a ketone (Compound #5)).

A mixture of the epoxide and the ketone compound was brominated to provide a Compound #6. Compound #6 was then processed to form the CDDO-Me compound #7. The CDDO-Me Compound #7 would then be processed to provide CDDO (Compound #8) with lithium iodide, and ultimately further reacted to a CDDO-EA Compound #10. (FIG. 19).

A detailed description of the chemical synthesis process for CDDO-EA from oleanolic acid, as depicted in FIG. 19, is provided in the following experimental section and series of steps.

Step 1-Synthesis of methyl (4aS,6aS,6bR,8aR,10S,12aR,12bR,14bS)-10-hydroxy-2,2,6a,6b,9,9,12a-heptamethyl-1,3,4,5,6,6a,6b, 7,8,8a,9,10,11,12,12a, 12b, 13,14b-octadecahydropicene-4a (2H)-carboxylate (2): K₂CO₃ (30.0 g, 21.7 mmol, 3.0 eq) was added potion wise to a stirred solution of oleanolic acid (1) (33.0 g, 72.3 mmol, 1.0 eq) in dimethyl formamide (300 ml). The reaction mixture was cooled down to 0° C. Iodomethane (11.3 g, 4.95 ml, 79.5 mmol, 1.1 eq) was added dropwise to the suspension mixture. After the completion of addition, the reaction was warmed up to room temperature and stirred overnight. After the completion of the reaction, dimethyl formamide was removed by distillation. The resulting solid was dissolved in 1 L of dichloromethane. The solution was washed with water (300 ml) four times and sat. NaCl aq. The organic layer was dried over Na₂SO₄, and the solvent was evaporated. 32.5 g of crude product 2 was obtained (96%) as a white solid, which was used for the next step without further purifications.

This step 1 is shown in the following Diagram 1:

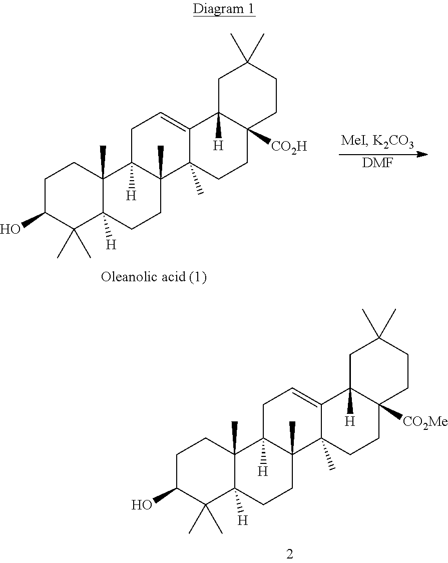

Step 2—Synthesis of methyl (4aS,6aS,6bR,8aR,12aR,12bR,14bS)-2,2,6a,6b,9,9,12a-heptamethyl-10-oxo-1,3,4,5,6,6a,6b,7,8,8a,9,10,12a, 12b, 13,14b-hexadecahydropicene-4a (2H)-carboxylate (3): Ester 2 (29.0 g, 61.6 mmol, 1.0 eq) obtained above was dissolved in anhydrous dimethyl sulfoxide (700 ml) at room temperature (r.t.). Fluorobenzene (12.3 ml) and iodoxybenzoic acid (51.7 g, 184.8 mmol, 3.0 eq) which was freshly prepared as described in the literature, were added to the solution. The resulting suspension was heated up to 85° C. under nitrogen and stirred for 16 hours. The reaction was cooled down and quenched with 20% sodium thiosulfate aq. (500 ml) and extracted with dichloromethane four times. The combined organic extracts were washed with sat. NaHCO₃ aq. and sat. NaCl aq., and then dried over Na₂SO₄. The solvent was removed to give the crude product 3 as yellowish solid. Purification by column chromatography (hexane:ethyl acetate=80:20) afforded 21.4 g of white foamy Compound 3 (75%).

This Step 2 is shown in the following Diagram 2:

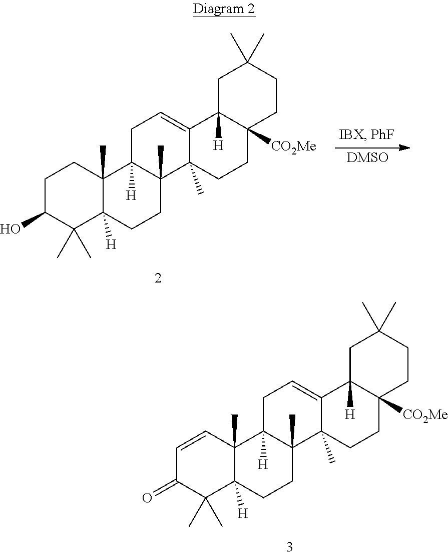

### Step 3-Synthesis of methyl (4aR,6aR,6bR,10aR,12aR,12bS,14aS)-3,3,6b, 10,10,12a, 12b-heptamethyl-9-oxo-2,3,4,4a, 6,6a, 6b,9,10,10a, 11,12,12a, 12b, 13,14-hexadecahydro-1H-piceno[12b, 13-b]oxirene-14a (5aH)-carboxylate (4)+methyl (4aS,6aR,6bR,8aR,12aR,12bR,14bS)-2,2,6a,6b,9,9,12a-heptamethyl-10,14-dioxo-1,3,4,5,6,6a,6b,7,8,8a,9,10,12a, 12b, 13,14,14a, 14b-octadecahydropicene-4a (2H)-carboxylate (5): Substrates

Enone 3 (21.4 g, 45.8 mmol, 1.0 eq.) was dissolved in methylene chloride (100 ml) and solution was cooled down to 0° C. m-Chloroperbenzoic acid (14.7 grams, 70% in water, 59.5 mmol, 1.3 eq) was added potion wise at 0° C. After the completion of addition, the reaction was warmed up to room temperature and kept stirring for overnight. The reaction mixture was diluted with methylene chloride (300 ml), and the resulting mixture was washed with 20% aqueous sodium thiosulfate three times, 10% potassium carbonate three times, and sat. NaCl aq. The organics were dried over Na₂SO₄ and the solvent was evaporated to afford 21.7 g crude mixture of 4 and 5 as white solid (98%), which was used directly for the next step without further purifications.

This Step 3 is shown in the following Diagram 3:

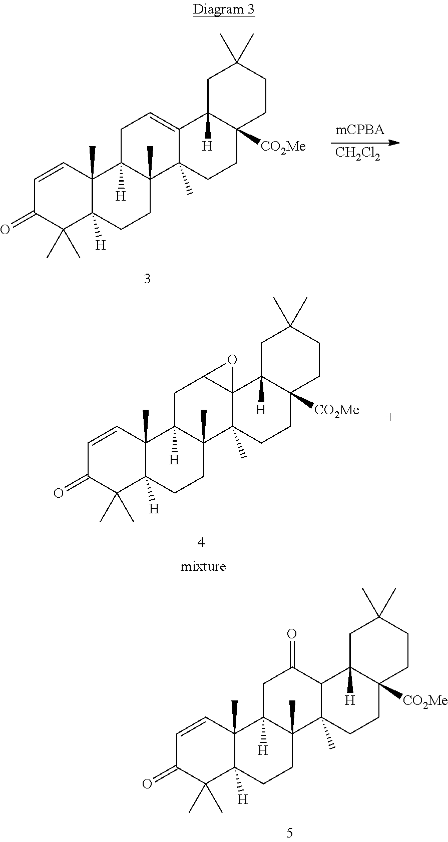

### Step 4—Synthesis of methyl (4aS,6aR,6bS,8aR,12aR,14bS)-11-bromo-2,2,6a, 6b,9,9,12a-heptamethyl-10,14-dioxo-1,3,4,5,6,6a,6b, 7,8,8a,9,10,12a, 14,14a, 14b-hexadecahydropicene-4a (2H)-carboxylate (6)

To a solution comprising Compound 4 and Compound 5 (substrates) (4.00 g, 8.30 mmol, 1.0 eq) in acetic acid (18 ml), a 47% HBr aq. (0.025 ml, 0.22 mmol, 0.026 eq) was added dropwise at room temperature, to form a reaction mixture. The reaction mixture was then heated to 35° C., and Br₂ (2.13 ml, 41.3 mmol, 5.0 eq) was added dropwise. The resulting reaction mixture was kept stirring for 1.5 h at the same temperature. Acetic acid was partially removed under vacuum. The residue was then quenched with 20% aqueous sodium thiosulfate, and extracted with dichloromethane four times. The combined organic extracts were washed with saturated sodium bicarbonate twice, brine, and dried over Na₂SO₄. The solvent was evaporated, and column chromatography (hexanes:ethyl acetate=80:20) gave 3.46 g (75%) of bromo enone 6 as a yellow solid.

This Step 4 is shown in the following Diagram 4:

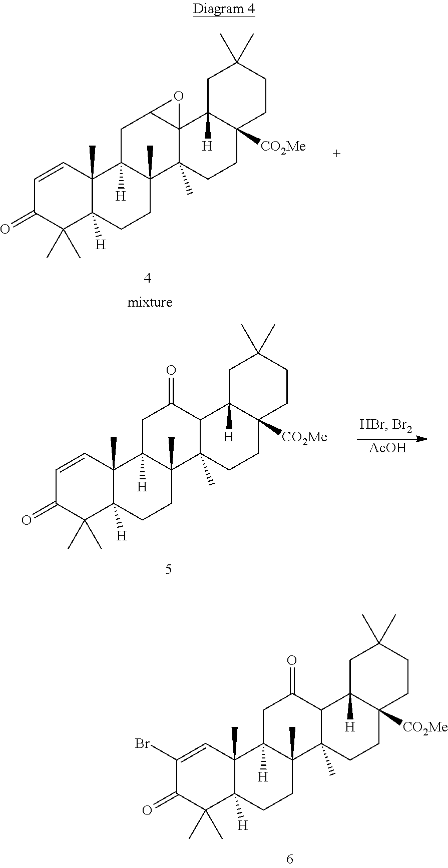

### Step 5—Synthesis of bardoxolone methyl (CDDO-Me): methyl (4aS,6aR,6bS,8aR,12aS,14bS)-11-cyano-2,2,6a,6b,9,9,12a-heptamethyl-10,14-dioxo-1,3,4,5,6,6a,6b,7,8,8a,9,10,12a, 14,14a, 14b-hexadecahydropicene-4a (2H)-carboxylate (7)

Bromo enone 6 (4.98 g mmol, 8.90 mmol 1.0 eq) is dissolved in anhydrous dimethyl formamide (50 ml) under nitrogen at room temperature. To the stirred solution, copper (I) cyanide (1.72 g, 9.79 mmol, 1.1 eq) and potassium iodide (566 mg, 1.78 mmol, 0.20 eq) were added, and the resulting reaction mixture was heated up to 120° C. and stirred for 24 h. The suspension was cooled to room temperature, quenched with water (200 ml), and diluted with ethyl acetate (500 ml). Formed cupper salts were removed by filtration before extraction. The organic phase was washed with saturated NaHCO₃ twice and sat. NaCl aq., and dried over Na₂SO₄. After evaporated the solvent, and column chromatography (hexanes:ethyl acetate=75:25) to give 3.82 g (85%) of bardoxolone methyl (7) as a yellowish solid.

This Step 5 is shown in the following Diagram 5:

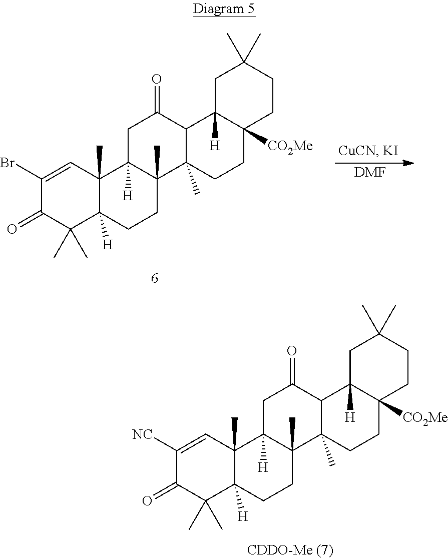

### Step 6—Synthesis of bardoxolone (CDDO), (4aS,6aR,6bS,8aR,12aS,14bS)-11-cyano-2,2,6a,6b,9,9,12a-heptamethyl-10,14-dioxo-1,3,4,5,6,6a,6b, 7,8,8a,9,10,12a, 14,14a, 14b-hexadecahydropicene-4a (2H)-carboxylic acid (8) 3

CDDO-Me (7) (3.82 g, 7.55 mmol, 1 eq) and dry LiI (18.71 g, 140 mmol, 18.5 eq) in dry DMF (10 ml) was heated under reflux for 4 h. The solution is quenched with 5% HCl aq. The mixture was extracted with EtOAc three times. The organic extract was washed with water three times, sat. NaCl aq., and dried over Na₂SO₄. The solvent was evaporated. Purification by column chromatography (dichloromethane only to dichloromethane:methanol=90:10) to give 3.56 g (96%) of CDDO (8).

This Step 6 is shown in the following Diagram 6

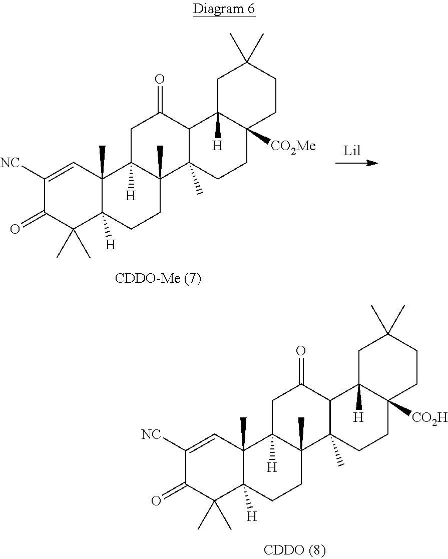

Step 7—Synthesis of 4aS,6aR,6bS,8aR,12aS,14bS)-11-cyano-2,2,6a,6b,9,9,12a-heptamethyl-10,14-dioxo-1,3,4,5,6,6a,6b,7,8,8a,9,10,12a, 14,14a, 14b-hexadecahydropicene-4a (2H)-carbonyl chloride (9) 4: A mixture of CDDO (8) (3.0 g, 6.1 mmol) and oxalyl chloride (8.73 g, 5.9 ml, 68.8 mmlol, 11.3 eq) in anhydrous dichloromethane (60 ml) was stirred at room temperature overnight. The solvent was evaporated, and the residue was co-evaporated with benzene three times. 3.66 g (99%) of crude 9 was obtained. This was used for next step without further purification.

This Step 7 is shown in the following Diagram 7:

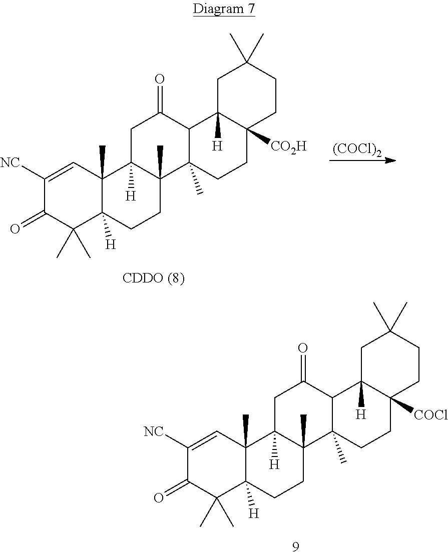

### Step 8—Synthesis of CDDO-EA, (4aS,6aR,6bS,8aR,12aS,14bS)-11-cyano-N-ethyl-2,2,6a,6b,9,9,12a-heptamethyl-10,14-dioxo-1,3,4,5,6,6a,6b, 7,8,8a,9,10,12a, 14,14a, 14b-hexadecahydropicene-4a (2H)-carboxamide (10)

The solution of Compound #9 (3.36 g, 6.59 mmol, 1.0 eq) in benzene (60 ml) was added to the solution of ethylamine hydrochloride (1.18 g, 14.4 mmol, 2.2 eq) and NaHCO₃ (3.0 g, 35.9 mmol, 5.5 eq) in water (60 ml). The mixture was stirred at room temperature overnight. The layers were separated, and the aqueous layer was extracted with benzene (60 ml). The combined organic layers were washed with sat. NaHCO₃aq, water, and sat. NaCl aq, and dried over Na₂SO₄. The solvent was evaporated. Purification was performed by column chromatography (dichloromethane only to dichloromethane:methanol=90:10) to give 2.45 g (72%) of CDDO-EA (10).

This Step 8 is shown in the following Diagram 8:

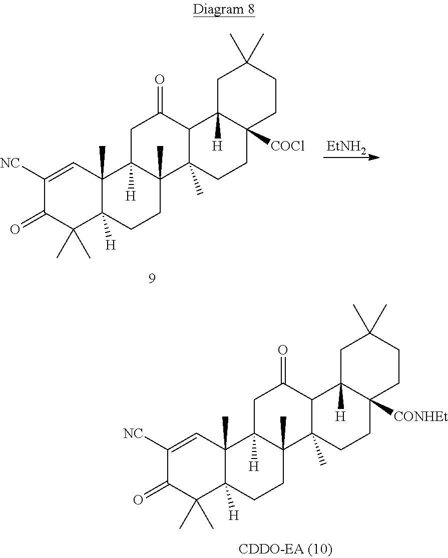

The new synthesis procedure disclosed provides for the use of a much reduced weight equivalent of catalyst, HBr (0.025 eq catalyst, Br₂ (reagent) (2.4 eq.)/(in solvent AcOH,) at 35° C.,1.5 hour), compared to the literature of Fu et al. 2013, that provides for a much higher weight equivalent catalyst amount HBr (0.44 eq) weight equivalent catalyst), Br₂ (2.4 eq), AcOH, 35° C., 24 hours). In addition, while the prior methods required at least 24 hours for chemical synthesis of bardoxolone methyl (CDDO-Me), the presently disclosed chemical synthesis pathway provides for a much shorter synthesis time of less than about 2 hours (about 1.5 hours) compared to the previous conventional reaction time of 24 hours.

The significantly reduced amount of acid catalyst required in the present synthesis scheme, the greater degree of specificity of reaction products (e.g., appropriate brominated compound intermediate), and the vastly shorter processing time (less than about 2 hours), provides a commercially-scalable technique suitable for providing sufficient yields of CDDO compounds and CDDO derivatives. These products include CDDO, CDDO-Me, and CDDO-EA products.

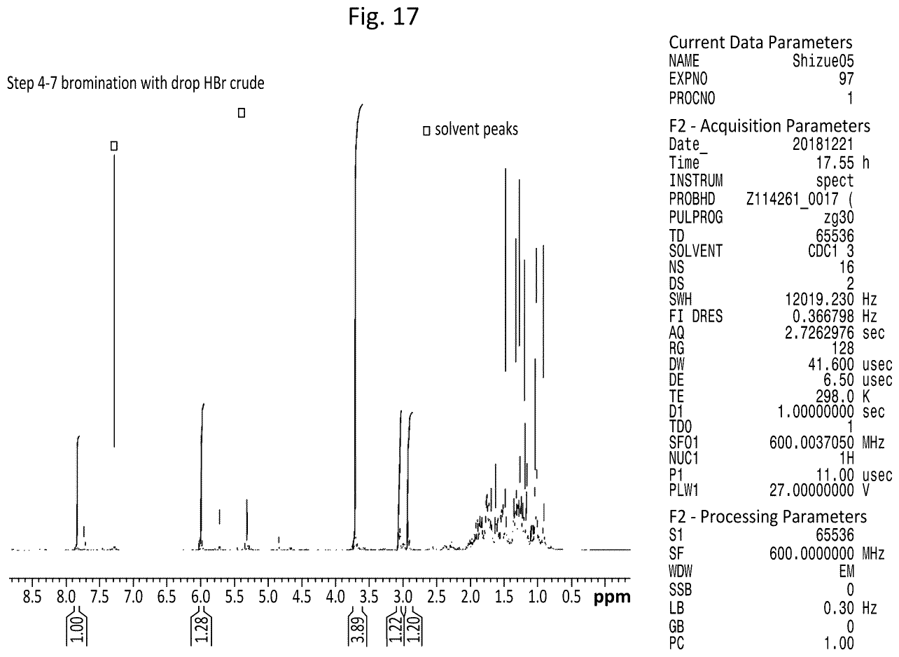

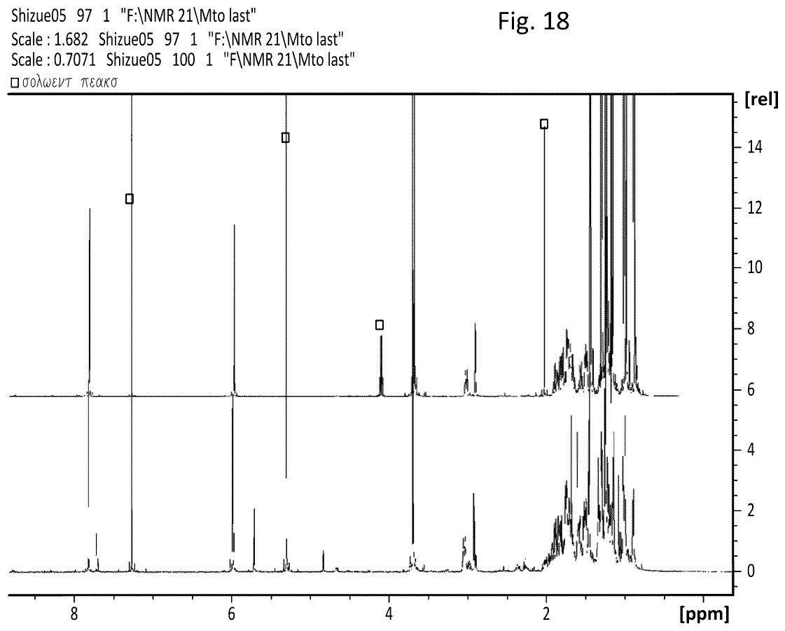

FIGS. 17 and 18 provide the spectral analysis of the products obtained according to the present synthesis techniques, and evidence the successful synthesis of a compound having the desired molecular structure for CDDO-Me.

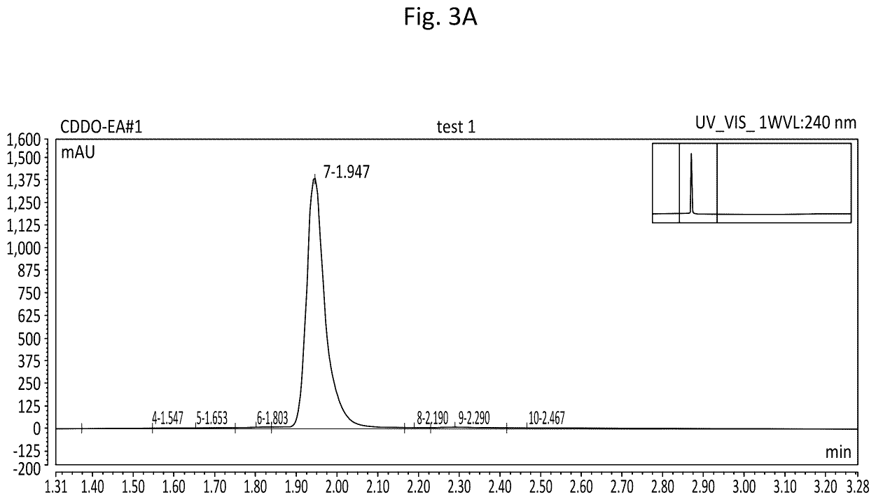

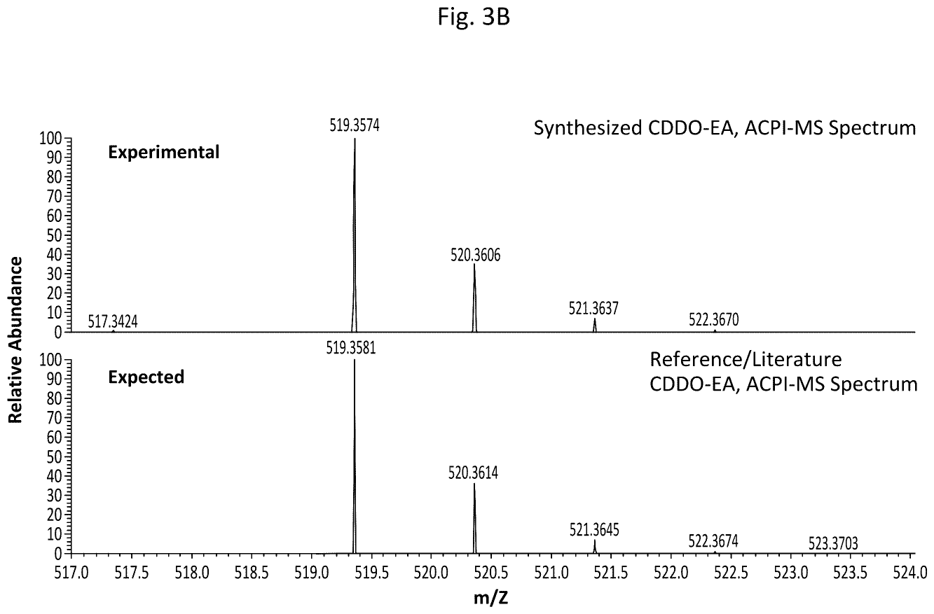

The product at each step was purified and the structure was confirmed by NMR spectroscopy. The product, CDDO-Me, was analyzed by HPLC and APCI-MS to determine the purity and confirm the molecular formula, and compared to the “Expected” mass spectroscopy as provided from the database MassHunter (Agilent). HPLC analysis showed that the CDDO-Me product prepared according to the presently modified and improved method was of a high purity (>99%) and APCI-MS showed that the product had the correct mass for the synthesized CDDO-Me (molecular formula: C₃₃H₄₆N₂O₃). (See FIG. 3). Approximately 8 g of CDDO-EA may be synthesized in about 6-8 weeks according to this procedure.

The modified and improved synthesis process developed is provided at FIG. 19. In contrast to prior art methods, including those of Fu et al. (2013), the intermediate product #6, which was required in order to progress to production of #7 (CDDO-Me), and then on to #8 (CDDO), was not possible according to the Fu et al. approach. Instead, an unsatisfactory over-bromination of material (Compound #11) would occur using the catalyst ratio of reagents specified by Fu et al. (See FIG. 20).

The intermediate product #6 was not provided by the Fu et al. process because, among other things, the ratio of catalyst to substrate eq was not appropriate, as it was several fold too high. What resulted when the catalyst eq and the substrate eq was attempted was the production of an over-brominated material. As shown in FIG. 20 attached, the “X” drawn through the chemical structure indicates that the appropriate brominated compound #6 was not produced. An unwanted undesired compound, labeled as compound 11 in FIG. 20, resulted instead. A product 11 that was over brominated occurred, as well as alpha-bromination. Upon performing an elimination reaction, a tetrasubstituted olefine resulted.

In the present synthesis, the bromination reaction to synthesize compound 6 will be carried out with a substrate comprising a mixture of epoxide (Compound #4) and ketone (Compound #5) protected compounds, using a much reduced equivalent amount of acid catalyst (in this case, HBr, 0.025 eq) (less than 5% (it is 2.5%) of the catalyst eq described in the Fu et al. literature, 0.44 eq.). The present synthesis technique provides a reaction that will produce the properly brominated #6 compound, and in a much more reduced amount of time (about 1.5 to 2 hours, compared to 24 hours).

### Example 2—CDDO Prevents Obesity and Inhibits Weight Gain In Vivo

The present example demonstrates the utility of the present methods and preparations for inhibiting weight gain, and in preventing obesity, in an animal, particularly in an animal consuming a high-fat diet. The present example also demonstrates the utility of the present invention for enhancing and/or facilitating weight loss in vivo.

The present example was conducted using CDDO-EA (Compound #10) synthesized according to the method described in Example 1, and as depicted in FIG. 19. The structure of the synthesized CDDO-EA was verified by comparison to a MassHunter (Agilent) database, generated structure.

To assess the protective effects of CDDO-EA from obesity and insulin resistance, C57B16/J mice (6-8 weeks old, male, strain #000664, The Jackson Laboratory) were used. The animals were fed a Low Fat diet (LFD) (10% total calories from fat, chow containing 0.04% CDDO-EA) or a High Fat diet (HFD) (60% of total calories from fat) incorporated with or without CDDO-EA (chow containing 0.04% CDDO-EA) for six weeks.

Feeding groups: The mice were put into one of four groups:
(i) HFD (60% total calories from fat),
(ii) HFD+0.04% CDDO-EA,
(iii) LFD (10% total calories from fat),
(iv) LFD+0.04% CDDO-EA.

Unlike Shin et al. and Camer et al., where the CDDO was administered via gavage or in the drinking water, the present studies provide for a feed material (veterinary chow mix) formulated to include 400 mg CDDO-EA in 1 kilogram of veterinary chow, which is 0.04% CDDO-EA in 1 kilogram of veterinary chow. It was concluded that the CDDO-EA in the veterinary chow material did not affect palatability of the chow to the animals. This is because the amount of chow without CDDO-EA (LFD) consumed by the animals was approximately the same amount of chow with CDDO-EA consumed by the animals (LFD+CDDO-EA) (See Table 3).

High Fat Diet (HFD) Without CDDO-EA (CDDO-EA, CDDO and CDDO analog and derivative Free):

60% Calories from Fat Diet (60/Fat) Chow

| Formula | g/Kg |
|---|---|
| Casein | 265.0 |
| L-Cystine | 4.0 |
| Maltodextrin | 160.0 |
| Sucrose | 90.0 |
| Lard | 310.0 |
| Soybean Oil | 30.0 |
| Cellulose | 65.5 |
| Mineral Mix, AIN-93-G-MX (94046) | 48.0 |
| Calcium Phosphate, dibasic | 3.4 |
| Vitamin Mix, AIN-93-VX (94047) | 21.0 |
| Choline Bitartrate | 3.0 |
| Blue Food Color | 0.1 |

Selected Nutrient Information

| | % by Weight | % kcal from |
|---|---|---|
| Protein | 23.5 | 18.3 |
| Carbohydrate | 27.3 | 21.4 |
| Fat | 34.3 | 60.3 |

Kcal/g 5.1
Values are calculated from ingredient analysis or manufacturer data

Key Features
+Purified Diet
+Diet Induced Obesity
+High Fat

High Fat Diet (HFD) with CDDO-EA:

60% Calories from Fat Chow + CDDO-EA Diet (400 ppm, R)

| Formula | g/Kg |
|---|---|
| Casein | 265.0 |
| L-Cystine | 4.0 |
| Maltodextrin | 160.0 |
| Sucrose | 90.0 |
| Lard | 310.0 |
| Soybean Oil | 30.0 |
| Cellulose | 65.1 |
| Mineral Mix, AIN-93-G-MX (94046) | 48.0 |
| Calcium Phosphate, dibasic | 3.4 |
| Vitamin Mix, AIn-93-VX (94047) | 21.0 |
| Choline Bitartrate | 3.0 |
| CDDO-EA | 0.4 |
| Blue Food Color | 0.1 |

Selected Nutrient Information
60% Calories from Fat- High fat Diet + CDDO-EA

| | % by Weight | % kcal from |
|---|---|---|
| Protein | 23.5 | 18.3 |
| Carbohydrate | 27.3 | 21.4 |
| Fat | 34.3 | 60.3 |

Kcal/g 5.1
Values are calculated from ingredient analysis or manufacturer data

Key Features
+Purified Diet
+CDDO-EA
+High Fat
+Color Coded Red

Low Fat Diet (LFD) with CDDO-EA:

10% Calories from Fat Chow + CDDO-EA Diet (400 ppm, G)

| Formula | g/KG |
|---|---|
| Casein | 210.0 |
| L-Cystine | 3.0 |
| Corn Starch | 464.6 |
| Maltodextrin | 100.0 |
| Sucrose | 90.0 |
| Lard | 20.0 |
| Soybean Oil | 20.0 |
| Cellulose | 37.15 |
| Mineral Mix, AIN-93-G-MX (94046) | 35.0 |
| Calcium Phosphate, dibasic | 2.0 |
| Vitamin Mix, AIN-93-VX (94047) | 15.0 |
| Choline Bitartrate | 2.75 |
| CDDO-EA | 0.4 |
| Yellow Food Color | 0.1 |

Selected Nutrient Information
10% Calories from Fat - Low Fat Diet + CDDO-EA

| | % by Weight | % kcal from |
|---|---|---|
| Protein | 18.6 | 20.5 |
| Carbohydrate | 62.5 | 69.0 |
| Fat | 4.2 | 10.5 |

Kcal/g 3.6
Values are calculated from ingredient analysis or manufacturer data

Key Features
+Purified Diet
+Low Fat Control
+CDDO-EA
+Color Coded Green

Low Fat Diet (LFD) without CDDO-EA. No CDDO-EA added to Animal Chow (CDDO-EA Free):

10% Calories from Fat Chow (400 ppm, G)

| Formula | g/KG |
|---|---|
| Casein | 210.0 |
| L-Cystine | 3.0 |
| Corn Starch | 464.6 |
| Maltodextrin | 100.0 |
| Sucrose | 90.0 |
| Lard | 20.0 |
| Soybean Oil | 20.0 |
| Cellulose | 37.15 |
| Mineral Mix, AIN-93-G-MX (94046) | 35.0 |
| Calcium Phosphate, dibasic | 2.0 |
| Vitamin Mix, AIN-93-VX (94047) | 15.0 |
| Choline Bitartrate | 2.75 |
| Yellow Food Color | 0.1 |

Selected Nutrient Information
10% Calories from Fat - Low Fat Diet

| | % by Weight | % kcal from |
|---|---|---|
| Protein | 18.6 | 20.5 |
| Carbohydrate | 62.5 | 69.0 |
| Fat | 4.2 | 10.5 |

Kcal/g 3.6
Values are calculated from ingredient analysis or manufacturer data

Mice were singly housed.

Data were statistically analyzed by One-Way ANOVA, Two-Way ANOVA with repeated measures, or Two-Way ANOVA with Tukey's multiple comparison test. Throughout the collection of blood samples, OGTTs, and measurement of bodyweight, mice were unrestrained and awake. Blood samples were collected via tail snip before start of experimental feeding and then every two weeks. Oral glucose tolerance tests (OGTTs) were performed after the six-week experimental feeding, and blood samples were collected via tail snip during the OGTT. For OGTT, mice were given 2 g of glucose/kg of body weight by gavage after a five hour fast (36). Glucose levels were measured using a glucometer (Contour), and insulin concentrations were measured using an ELISA (Mercodia). Mice were weighed once a week. Glucose and insulin serum levels were measured every two weeks.

The data on the animal weights during the treatment is provided in the following Table 1:

TABLE 1

| | Average Weight (g) of Mice at Each Timepoint (weeks) | | | | | | |
|---|---|---|---|---|---|---|---|
| Mouse Group | 0 | 1 | 2 | 3 | 4 | 5 | 6 |
| LFD | 23.3 ± 0.8 | 24.3 ± 0.7 | 25.0 ± 0.6 | 26.2 ± 0.9 | 25.7 ± 1.0 | 27.2 ± 1.2 | 26.9 ± 1.3 |
| LFD + CDDO-EA | 25.5 ± 1.0 | 24.4 ± 0.9 | 24.4 ± 0.8 | 25.8 ± 0.7 | 25.4 ± 0.7 | 26.4 ± 0.6 | 26.0 ± 0.4 |
| HFD | 24.3 ± 0.5 | 27.0 ± 0.6 | 28.5 ± 0.8 | 31.5 ± 1.0 | 33.3 ± 1.0 | 35.9 ± 0.9 | 37.9 ± 1.0* |
| HFD + CDDO- | 23.8 ± 0.5 | 23.4 ± 0.5 | 23.2 ± 0.5 | 24.6 ± 0.5 | 24.1 ± 0.5 | 25.4 ± 0.5 | 25.3 ± 0.5** |
| EA | | | | | | | |

C57Bl6/J (6-8 weeks old, male) mice were fed low fat diet (LFD) or high fat diet (HFD) with or without CDDO-EA (diet containing 0.04% CDDO-EA) for 6 weeks (n = 5-7).
Data are represented as mean ± SEM (Two-Way Repeated Measures ANOVA).
*p < 0.0001 LFD vs. HFD;
**p < 0.0001 HFD vs. HFD + CDDO-EA.

1. Animals consuming the high fat diet without CDDO EA for 6 weeks were about 64.1% heavier compared to their starting weight at the end of the 6 week feeding period. Animals receiving the high fat diet with CDDO-EA for 6 weeks did not have a significant increase in body weight compared to their starting weight after the 6-week feeding period.
2. The high fat diet group gained 71% more weight than the low fat diet group.
3. There is a 66.8% difference in weight between mice fed a high fat diet and mice fed a high fat diet incorporated with CDDO-EA (0.04% CDDO-EA).
4. Significant weight gain was prevented by 66.8% in mice fed a high fat diet incorporated with CDDO-EA (0.04% CDDO-EA).

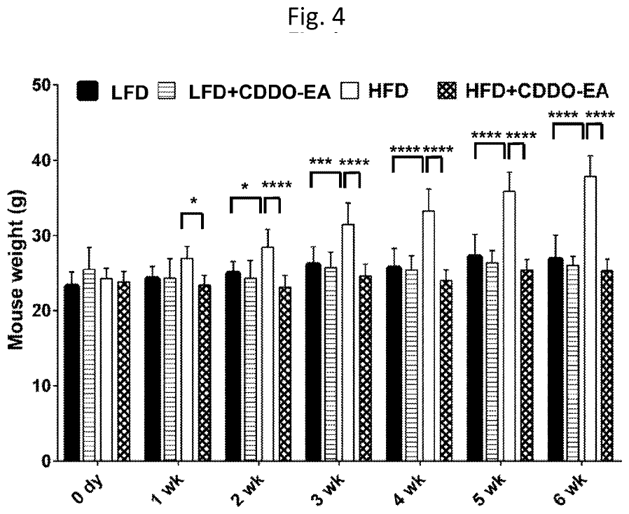

These results demonstrate that mice fed a HFD gained a significantly greater amount of weight than the animals fed a LFD which was evidenced each week, beginning at week two, and this was consistent throughout the six-week study. The incorporation of CDDO-EA in the HFD significantly blocked gain weight compared to mice fed a HFD alone (FIG. 4).

These data show that mice fed a HFD gained significantly more weight than the LFD fed animals by week two, and this was consistent throughout the six-week study. The incorporation of CDDO-EA in the HFD prevented the excess weight gain in mice fed a HFD alone (4).

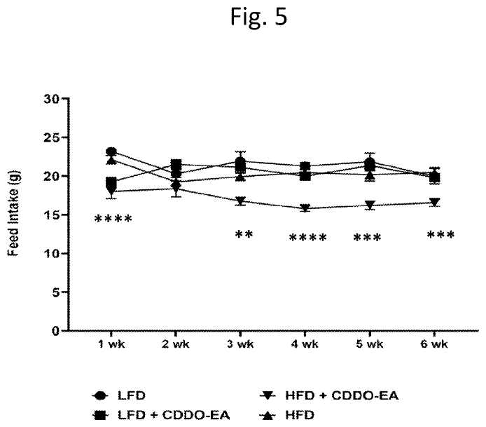

Energy intake was also measured. Energy intake is determined as a measure of the amount of food consumed by a subject (such as a subject mouse). It was found that animals provided a high fat diet (HFD) food chow mixed with CDDO-EA had a decreased energy intake (caloric food consumption) (less food volume by weight consumed) (FIG. 5). Thus CDDO-EA protects from development of HFD-induced obesity in vivo.

### Example 3—Oral CDDO EA Protects Against Hyperglycemia and Hyperinsulinemia

The present example was conducted using CDDO-EA synthesized according to the method described in Example 1, and as depicted in FIG. 19.

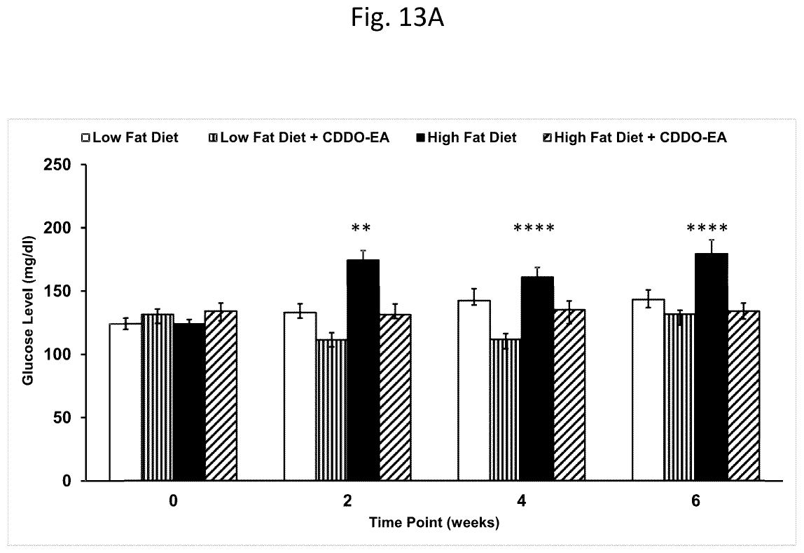

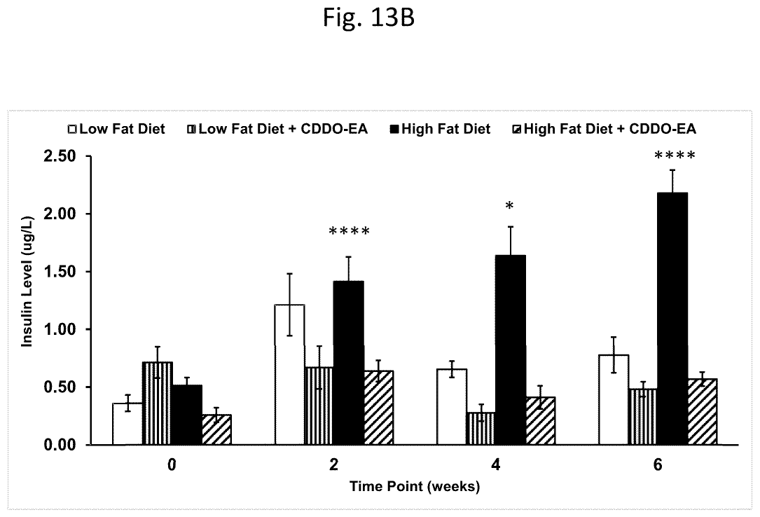

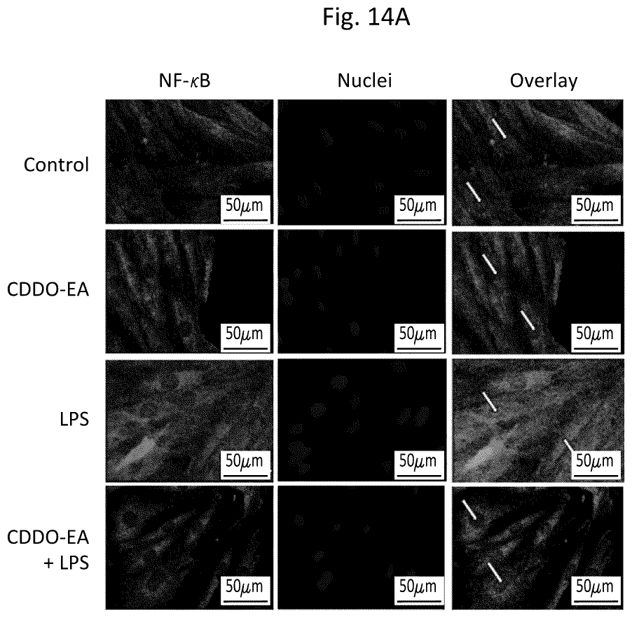

Glucose and insulin levels were measured in the subject animals to determine if prevention of obesity by CDDO-EA in a high fat diet (HFD) chow regimen and in a low fat diet (LFD) regimen coincided with a reduction in hyperglycemia and hyperinsulinemia. It is demonstrated here that serum glucose levels were significantly increased in mice fed only a HFD (CDDO-EA-free) at 2 weeks and remained significantly increased throughout the rest of the feeding (FIG. 13A).

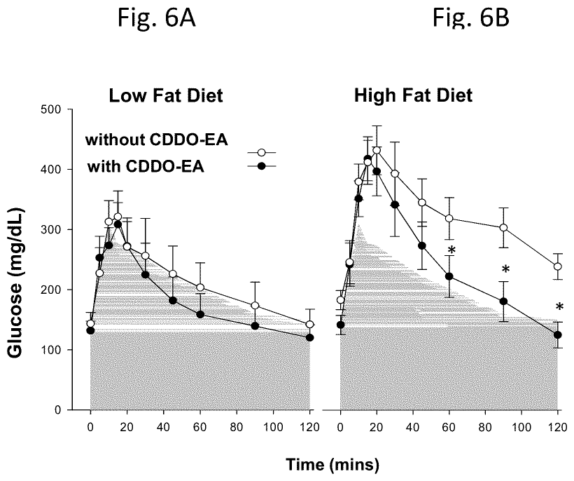

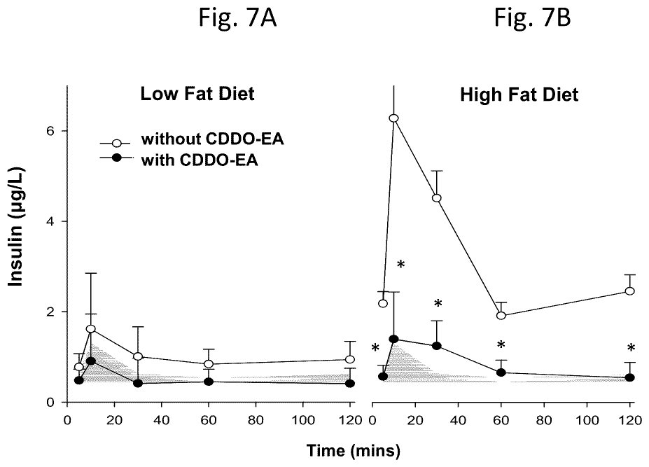

Serum glucose levels in mice fed a HFD with CDDO-EA did not increase throughout the six-week feeding (FIG. 13A). Serum insulin levels were significantly higher at 2 weeks and remained significantly higher in mice fed only a HFD compared to mice fed a HFD with CDDO-EA (FIG. 13B). OGTT showed that CDDO-EA prevented increased serum glucose (FIG. 6B) and insulin concentrations (FIG. 7B) in mice fed a HFD. In FIGS. 6A, 6B, 7A and 7B, the gray shaded area below each curve corresponds to the serum glucose levels and insulin levels before experimental feeding, respectively.

The serum glucose levels of both the LFD and LFD+CDDO-EA groups (left panel FIG. 6A) overlap with the serum glucose levels before experimental feeding, demonstrating no significant increase or decrease in serum glucose levels. This demonstrates that CDDO-EA as part of a low fat diet, in a non-obese animal, does not interfere with the maintenance of a normal, non-obese, healthy weight of the animal.

The HFD group's glucose levels (right panel FIG. 6B) were significantly higher after the 6-week HFD feeding. The HFD+CDDO-EA group's glucose levels overlap with the HFD up to the 45 min timepoint, and then significantly decrease to glucose levels before the HFD experimental feeding. In FIG. 7B, the gray shaded area below the curve corresponds to the insulin levels in these animals before the HFD experimental feeding. The insulin levels of LFD and LFD+CDDO-EA after the 6-week experimental feeding overlap with the insulin levels before experimental feeding (left panel FIG. 7A). The insulin levels of the HFD group (right panel FIG. 7B) are significantly higher after the 6-week HFD feeding.

The HFD+CDDO-EA group insulin levels did not increase significantly. They did overlap with the pre-regimen feeding insulin levels (i.e., before experimental feeding). These data show that CDDO-EA did not affect glucose and insulin levels of mice fed LFD with CDDO-EA. These results also demonstrate that a feeding of a CDDO-free HFD to an animal induces glucose intolerance and hyperinsulinemia in the animal, and significant weight gain. However, a feeding of a HFD containing CDDO-EA protected the subject animals against and/or inhibited an increase in glucose and insulin levels.

The present findings and data demonstrate that CDDO-EA prevents hyperglycemia, hyperinsulinemia, and glucose intolerance at least in part due to the prevention of obesity. These data also demonstrate that CDDO-EA will inhibit and/or prevent insulin resistance, and therefore is an effective health management tool to block and/or reduce the risk of the progression of an animal to type 2 diabetes (T2D) in vivo.

### Example 4—CDDO-EA Blocks LPS-Induced NF-κB Activation and Inflammation in Skeletal Muscle

The present example demonstrates the utility of the present invention for regulating skeletal muscle inflammation, and methods for preventing skeletal tissue inflammation. Methods for maintaining normal skeletal muscle metabolism and function are also demonstrated.

The CDDO-EA employed in the present skeletal muscle cell study, was synthesized according to the methods taught by Honda et al., (1998); Honda et al., (2000b); Honda et al., 2002), Yates et al. (2007), and U.S. Pat. Nos. 6,326,507 and 6,974,801, which are all incorporated herein by reference in their entirety.

The structure of the CDDO-EA compound used was verified by mass spectrographic comparison with a reference structure for the Compound generated from the database, MassHunter (Agilent).

NF-κB production is activated in skeletal muscle cells when exposed to LPS, resulting in the expression and production of pro-inflammatory cytokines. CDDOs are shown to inhibit inflammation and pro-inflammatory signaling pathways in skeletal muscle in the present study. CDDO-EA and CDDO-trifluoroethyl amide (TFEA) on skeletal muscle in N171-82Q mice, a transgenic mouse model of Huntington's disease, had been examined. CDDO-EA and CDDO-TFEA improved muscle strength and balance and increased survival and was shown to accumulate in the skeletal muscle. This indicates that a local effect by CDDO-EA and CDDO-TFEA had occurred. The effect of CDDOs on skeletal muscle, the primary site of whole-body glucose disposal, is demonstrated in the present study.

The present example demonstrates the effects of CDDO-EA to reduce lipopolysaccharide (LPS)-induced inflammation in skeletal muscle cells. To examine the anti-inflammatory properties of CDDO-EA in skeletal muscle cells, CDDO-EA was examined for its effect on nuclear factor-kappa B (NF-κB) activity in in L6-GLUT4myc muscle cells, in the context of LPS-mediated inflammatory responses in L6-GLUT4myc muscle cells. All experiments were independently repeated one to four times and done in duplicate or triplicate, and data were statistically analyzed by One-Way ANOVA or unpaired t-test, two tailed.

The L6-GLUT4myc rat myoblast cell line. L6-GLUT4myc myoblasts were maintained and differentiated to myotubes as previously described. L6-GLUT4myc rat myoblast cells were not used beyond passage 11. L6-GLUT4myc myoblasts stably express GLUT4myc glucose transporter. Myc epitope is in an exofacial loop of GLUT4. Antibiotic resistance for GLUT4myc stable transfection is blasticidin, and blasticidin is included in the growth media to maintain selection pressure. The L6-GLUT4myc cells are routinely monitored for mycoplasma.

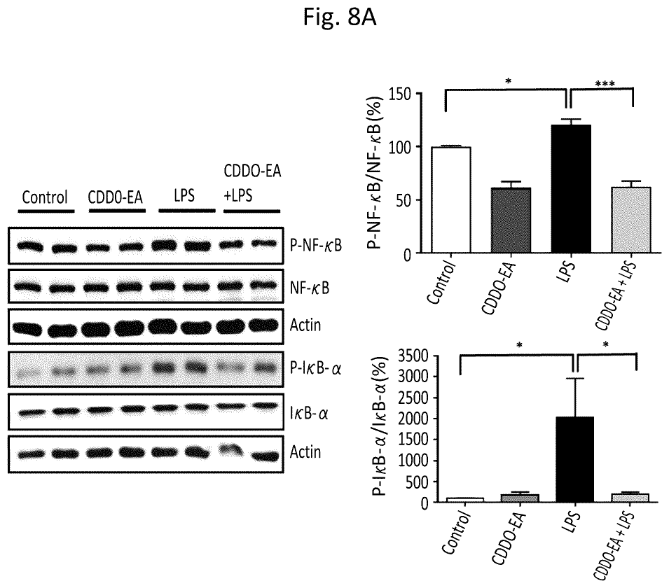

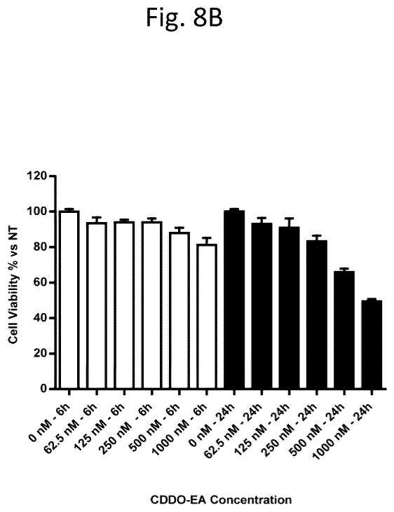

Cells were studied with vehicle alone [0.125% dimethyl sulfoxide (DMSO)] and LPS with and without CDDO-EA. Western blot analysis show that CDDO-EA decreased LPS-induced NF-κB and nuclear factor of kappa light polypeptide gene enhancer in B-cells inhibitor-alpha (IκB) phosphorylation (FIG. 8A). To determine if CDDO-EA inhibited NF-κB transcriptional activity, a luciferase assay was performed in which L6-GLUT4myc myotubes were transfected with a NF-κB luciferase reporter plasmid.

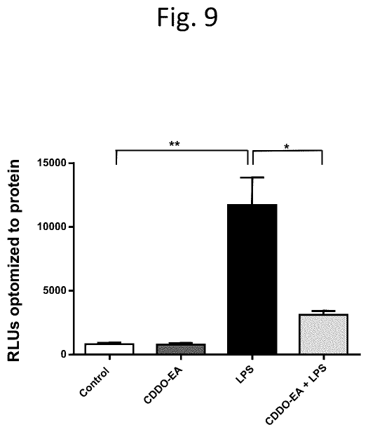

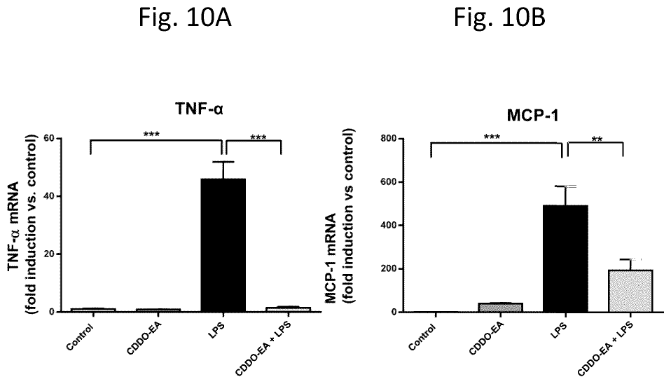

The results show that CDDO-EA prevented the promoter activity of NF-κB when myotubes are exposed to LPS (FIG. 9). To examine downstream signaling from NF-κB, mRNA expression of monocyte chemotactic protein-1 (MCP-1) and TNF-α in myotubes was assessed. NF-κB induces the transcription of these two pro-inflammatory mediators, which are implicated in the pathogenesis of insulin resistance and T2D. CDDO-EA lowered LPS-induced mRNA levels of TNF-α and MCP-1 in myotubes (FIG. 10A-10B).

These presented findings show that CDDO-EA demonstrates anti-inflammatory properties in the skeletal muscle. These findings demonstrate a novel biological property of CDDOs in regulating pro-inflammatory responses in the skeletal muscle, a central player in shaping whole-body insulin resistance, especially during obesity. Therefore, CDDO-EA provides for a treatment to prevent or inhibit the development of type 2 diabetes in an animal.

### Example 5—CDDO Inhibits LPS-Induced Cytokine and Chemokine Production in Macrophages; Role in Type 2 Diabetes

The present example demonstrates the utility of the present invention for inhibiting cytokine and chemokine production in a macrophage cell.

The CDDO-EA employed in the present example was prepared according to methods described in the art. Specifically, the CDDO-EA employed in the present studies on skeletal muscle cells was synthesized according to the methods taught by Honda et al., (1998); Honda et al., (2000b); Honda et al., 2002), Yates et al., (2007), and U.S. Pat. Nos. 6,326,507 and 6,974,801, which are all incorporated herein in their entirety.

The structure of the CDDO-EA compound used was verified by mass spectrographic comparison with a reference structure for the Compound generated from the database, MassHunter (Agilent).

To examine the anti-inflammatory properties of CDDO-EA, CDDO-EA was examined and determined to block cytokine and chemokine production in macrophages. The production of TNF-α was examined because of the critical role this cytokine plays in the development of insulin resistance and type 2 diabetes (T2D).

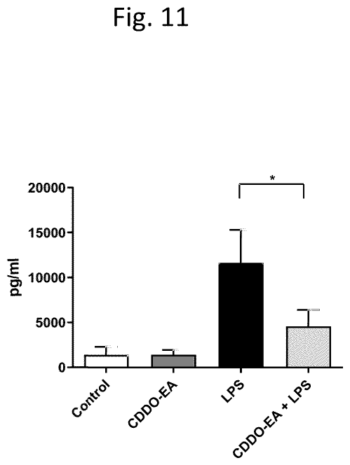

As seen in FIG. 11, in RAW 264.7 mouse macrophages, LPS induced a dramatic rise in production of TNF-α. The rise in TNF-α was substantially blocked by pre-treatment with CDDO-EA. To further examine the properties of CDDO-EA in blocking expression of pro- and anti-inflammatory cytokines and chemokines in macrophages, THP-1 human macrophages were exposed to CDDO-EA prior to LPS treatment. Cytokines and chemokines were measured by a bio-plex assay.

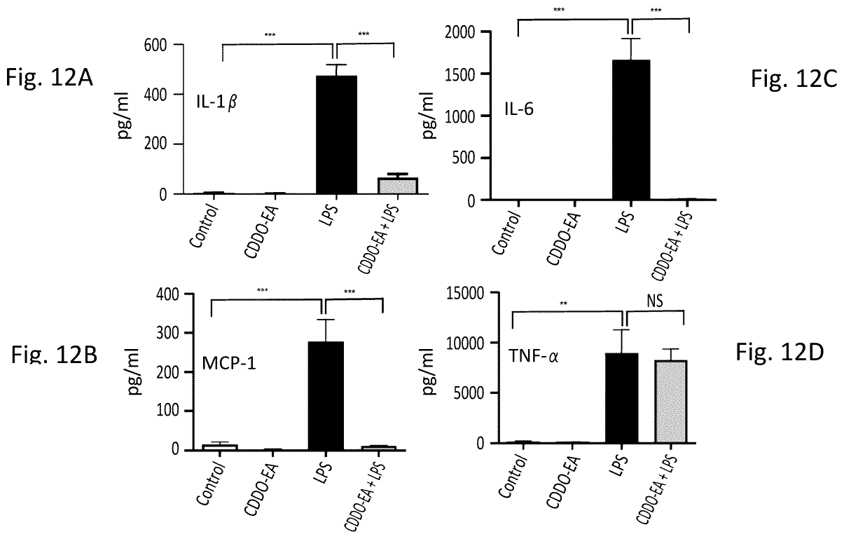

Table 2 and FIG. 12 show that CDDO-EA primarily inhibited LPS-induced IL-1B, IL-6 and MCP-1 production in THP-1 human macrophages.

TABLE 2
Human cytokine/chemokine panel in THP-1 macrophages exposed to CDDO-EA and LPS.

| Cytokine/ | | | | |
|---|---|---|---|---|
| Chemokine | | | | CDDO-EA + |
| (pg/ml) | Control | CDDO-EA | LPS | LPS |
| IL-1β | 4.43 ± 2.48 | 2.19 ± 1.49 | 472.76 ± 5.79***^{¥} | 64.65 ± 17.12***^{§} |
| IL-2 | ND | ND | 5.25 ± 0.69 | 4.09 ± 1.51 |
| IL-4 | 0.27 ± 0.14 | 0.21 ± 0.12 | 1.63 ± 0.33 | 1.55 ± 0.18 |
| IL-5 | ND | ND | ND | ND |
| IL-6 | 0.39 ± 0.24 | 0.12 ± 0.02 | 1658.65 ± 60.12***^{¥} | 12.67 ± 5.50***^{§} |
| IL-7 | ND | ND | ND | ND |
| IL-8 | 8591.50 ± 2114.62 | 6339.67 ± 1535.18 | 5815.00 ± 2259.42 | 9691.33 ± 1268.42 |
| IL-10 | ND | ND | 10.19 ± 5.01 | ND |
| IL-12 | ND | ND | 0.92 ± 0.48 | 2.00 ± 1.00 |
| IL-13 | ND | ND | ND | ND |
| IL-17 | 10.99 ± 7.13 | 22.72 ± 9.47 | 54.04 ± 11.10*^{¥} | 40.16 ± 8.13 |
| G-CSF | ND | ND | 13.73 ± 4.52 | 10.72 ± 4.71 |
| GM-CSF | ND | ND | ND | ND |
| IFN-γ | 7.37 ± 4.08 | 24.78 ± 20.55 | 154.37 ± 57.80*^{¥} | 114.38 ± 5.43 |
| MCP-1 | 14.86 ± 7.07 | 2.11 ± 1.35 | 277.16 ± 56.96***^{¥} | 11.05 ± 1.65***^{§} |
| MIP-1β | 7140.33 ± 1891.60 | 3763.83 ± 1229.25 | 10273.33 ± 1249.85 | 11212.50 ± 877.01 |
| TNF-α | 161.17 ± 65.47 | 128.33 ± 38.63 | 8914.50 ± 2358.9**^{¥} | 8237.33 ± 1137.80 |

THP-1 macrophages were pre-treated with 500 nM CDDO-EA for 1 hr then exposed to 100 ng/ml LPS for 6 hr. Cytokine and chemokine levels were measured by a Bio-Plex Pro-Human 17-plex ELISA kit. Experiment was run in triplicate. Data represented as mean ± SEM.
*p < 0.05;
**p < 0.01;
***p < 0.001;
^{¥}compared to Control;
^{§}compared to LPS.
ND, not detected.

As shown in FIG. 12A-D, CDDO-EA inhibits the production of pro-inflammatory cytokines and chemokines in macrophages.

### Example 6—CDDO Alleviates LPS-Induced NF-κB and IκB-α Phosphorylation

The CDDO-EA employed in the present studies in skeletal muscle cells was prepared according to methods described in the art. Specifically, the CDDO-EA employed in the present studies on skeletal muscle cells was synthesized according to the methods taught by Honda et al., (1998); Honda et al., (2000b); Honda et al., 2002), Yates et al., (2007), and U.S. Pat. Nos. 6,326,507 and 6,974,801, which are all incorporated herein in their entirety.

The structure of the compound used was verified by mass spectrographic comparison with a reference structure for the Compound generated from the database, MassHunter (Agilent).

The anti-inflammatory properties of CDDO-EA on skeletal muscle was examined. Specifically, the anti-inflammatory effect of CDDO-EA in inhibiting NF-κB activation in skeletal muscle is demonstrated.

CDDO-EA inhibited LPS-induced NF-κB phosphorylation in L6-GLUT4myc myotubes (FIG. 8A).

As shown in FIG. 8A, CDDO-EA also inhibited the LPS-induced phosphorylation of IκB-α in myotubes.

To determine if 500 nM CDDO-EA affected cell viability, myotubes were treated with increasing doses of CDDO-EA for 6 h and 24 h. Myotubes treated for 6 hours with 500 nM CDDO-EA showed 92% viability (FIG. 8B). At 24 hours, the cell viability with 500 nM CDDO-EA was 62%.

Cumulatively, these data show that CDDO-EA blocks LPS-induced activation of the IKK/IκB-α/NF-κB signaling pathway and that CDDO-EA at 500 nM does not induce cell death in skeletal muscle cells at 6 hours.

### Example 7—Anti-Inflammatory Action of CDDO-CDDO Blocks LPS-Induced NF-κB Nuclear Translocation in Skeletal Muscle Cells

The present example demonstrates that CDDO-EA was effective in blocking NF-κB translocation to the nucleus of skeletal muscle cells.

The CDDO-EA employed in the present studies on skeletal muscle cells was synthesized according to the methods taught by Honda et al., (1998); Honda et al., (2000b); Honda et al., 2002); Yates et al., (2007); and U.S. Pat. Nos. 6,326,507 and 6,974,801, which are all incorporated herein in their entirety.

The structure of the compound used was verified by mass spectrographic comparison with a reference structure for the Compound generated from the database, MassHunter (Agilent).

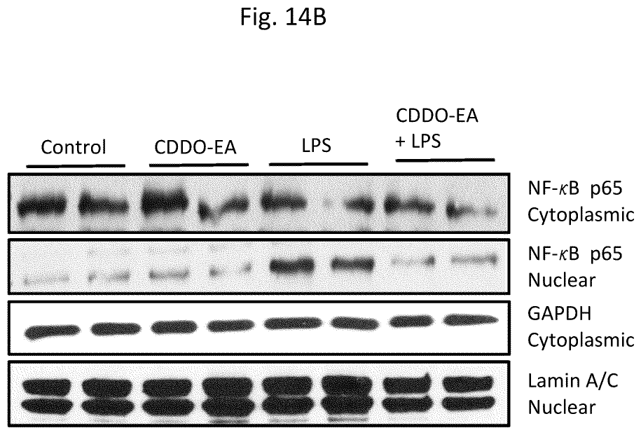

NF-κB is bound to IκB-α in the cytoplasm. Upon activation of the IKK/NF-κB/IκB pathway, IκB is degraded, freeing NF-κB to translocate into the nucleus and activate the transcription of pro-inflammatory mediators. To visualize the localization of NF-κB, immunofluorescence and confocal microscopy studies were performed in L6-GLUT4myc myo-tubes pre-treated with CDDO-EA for 1 h and then exposed to LPS for 1 h. The untreated muscle cells show a basal-level expression of p65 NF-κB in the nucleus (FIG. 14A). LPS treatment for 1 h increases nuclear staining of p65 NF-κB, while pre-treatment with CDDO-EA reduces LPS-induced p65 NF-κB nuclear staining.

Nuclear translocation of p65 NF-κB was examined by Western analysis of the nuclear and cytoplasmic fractions in CDDO-EA treated myo-tubes (FIG. 14B). LPS exposure of 1 h increased nuclear localization of p65 NF-κB. However, treatment of myotubes with CDDO-EA for 1 h prior to LPS exposure decreased both cytoplasmic and nuclear localization of p65 NF-κB.

Detailed description: To visualize the localization of NF-κB, immunofluorescence and confocal microscopy studies were performed in skeletal muscle cells (rat skeletal muscle cell line L6-GLUT4-myc) (FIG. 14A). The panels in the first vertical row (NF-κB), show NF-κB staining in cells from each of the four groups examined. (green). The panels in the second vertical row (“Nuclei”) show nuclei in the cells of each of the treatment groups examined stained a second color (blue). The third vertical row of panels (“Overlay”) show the merger of the far left (NF-κB) and center (“Nuclei”) vertical rows of panels. The first horizontal row of panels (“Control”) is the “control” group of skeletal muscle cells (not treated with CDDO-EA and LPS). This group of panels show the basal staining when there is no induced activation of NF-κB. The second horizontal row of panels (“CDDO-EA”) illustrates images of skeletal muscle cells treated with CDDO-EA alone and demonstrate that CDDO-EA had no effect on NF-κB activation. The third row of horizontal panels (“LPS”) illustrate images of skeletal muscle cells treated with LPS. LPS activates NF-κB translocation to the nuclei. The arrows in the last panel of this horizontal row (“LPS”), point to the nuclei of the skeletal cells, and the resulting pattern of staining demonstrates that the nuclei of these skeletal cells contained an increase in (green) staining (arrows), compared to the Control panel (“Control” horizontal row, third panel, arrows) and CDDO-EA (“CDDO-EA” horizontal row, third panel, arrows).

The bottom panel of the third vertical row of panels (FIG. 14A, “Overlay” vertical row, bottom panel) illustrates skeletal muscle cells which were pretreated with CDDO-EA, and then LPS. The images demonstrate that CDDO-EA prevented NF-κB translocation to the nucleus (“Overlay” vertical row, bottom panel, arrows point to areas showing decreased (green) staining (arrows) in the nuclei), compared to the LPS only treated group (“Overlay” vertical row, third panel). FIG. 14B shows the protein content of NF-κB in the cytoplasm and nuclei of skeletal muscle cells (rat skeletal muscle cell line L6-GLUT4-myc). The first two vertical bands are the control group (no CDDO-EA and no LPS), the next two vertical bands (#3 and #4) are the group treated with CDDO-EA only. The vertical bands #5 and #6 are the group treated with LPS. The vertical bands #7 and #8 are the group pretreated with CDDO-EA and then exposed to LPS. LPS treatment induced the translocation of NF-κB to the nucleus which is demonstrated by the high protein content (dark bands #5 and #6) in the nuclear fraction (labeled NF-κB p65 nuclear), compared to control group and CDDO-EA group. When skeletal muscle cells are pretreated with CDDO-EA and then exposed to LPS, CDDO-EA prevents the translocation of NF-κB to the nucleus (second horizontal row of bands), indicated by the decreased NF-κB protein content (lighter bands, #7 and #8, second horizontal row of bands, NF-κB p65 nuclear). The third horizontal row of bands are the proteins to verify cytoplasmic (GAPDH) fractions, and the fourth horizontal row of bands are proteins to verify nuclear (lamin) fractions. With the use of two methodologies, it is shown that CDDO-EA prevents LPS-induced activation of NF-κB in skeletal muscle cells by preventing the NF-κB translocation to the nucleus.

### Example 8—NF-κB Activity and TNF-α and MCP-1 Gene Expression are Inhibited by CDDO-EA in Skeletal Muscle Cells Exposed to LPS

The present example presents CDDO-EA suppresses LPS-induced inflammation in macrophages and myocytes and demonstrates the utility of CDDO-EA as a useful agent for protecting skeletal muscle from inflammation. In this manner, a useful treatment for type 2 diabetes with CDDO-EA is provided.

The CDDO-EA employed in the present studies on skeletal muscle cells was synthesized according to the methods taught by Honda et al., (1998); Honda et al., (2000b); Honda et al., 2002); Yates et al., (2007); and U.S. Pat. Nos. 6,326,507 and 6,974,801, which are all incorporated herein in their entirety.

The structure of the compound used was verified by mass spectrographic comparison with a reference structure for the Compound generated from the database, MassHunter (Agilent).

Skeletal muscle is the major site of insulin-induced glucose uptake. Insulin binds to its receptor on the skeletal muscle, initiating a signaling cascade resulting in glucose uptake. Insulin resistance in skeletal muscle is mainly due to impaired insulin signaling and glucose transport and is a hallmark of type 2 diabetes. The exact mechanism underlying insulin resistance in the skeletal muscle is not understood, but evidence indicates that inflammation occurs in the skeletal muscle which can contribute to insulin resistance. Chronic inflammation may be a result of high circulating levels of lipopolysaccharide (LPS) that can result from a change in gut microflora associated with obesity. Elevated LPS plasma levels are found in insulin-resistant obese and type 2 diabetic individuals, and this is negatively correlated with skeletal muscle insulin sensitivity. LPS induces the activation of the pro-inflammatory nuclear factor kappa B (NF-κB) signaling cascade. NF-κB is in the cytoplasm of cells. Upon activation, NF-κB translocates to the nucleus and activates the transcription of pro-inflammatory mediators.

To determine if CDDO-EA prevents the LPS-induced translocation of NF-κB, skeletal muscle cells (rat skeletal muscle cell line L6-GLUT4-myc) were pretreated with CDDO-EA and then exposed to LPS.

To determine the effects of CDDO-EA on LPS-induced NF-κB transcriptional activity in L6-GLUT4myc myotubes, a luciferase reporter under the control of a promoter containing NF-κB transcription binding sites was used.

LPS caused a 14.4-fold increase in NF-κB activity in myotubes at 6 h exposure (FIG. 9). Pre-treatment of myotubes with CDDO-EA significantly inhibited the LPS-induced NF-κB activity.

CDDO-EA blocked the mRNA expression of LPS-induced TNF-α and MCP-1 (FIGS. 10A and 10B). TNF-α and MCP-1 are pro-inflammatory mediators and are recognized to be involved in the progression and development of T2D and transcribed by NF-κB. Therefore, the CDDO-EA activity demonstrated here proves that the CDDO compounds will be useful for inhibiting inflammation in skeletal muscle cells and skeletal muscle tissues.

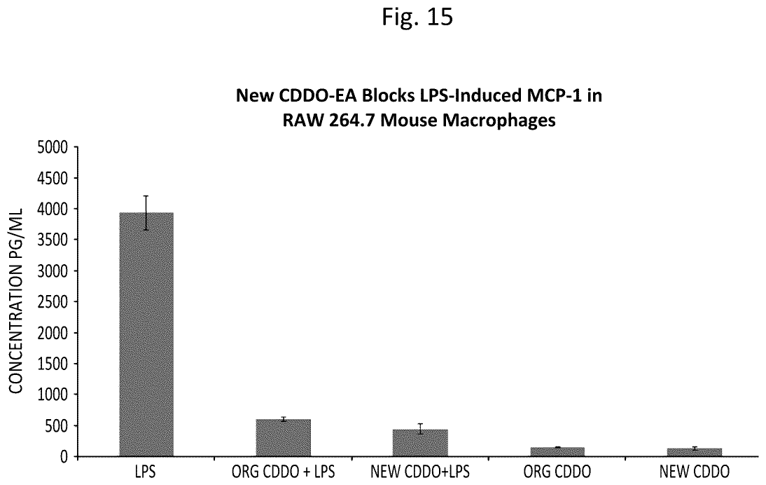

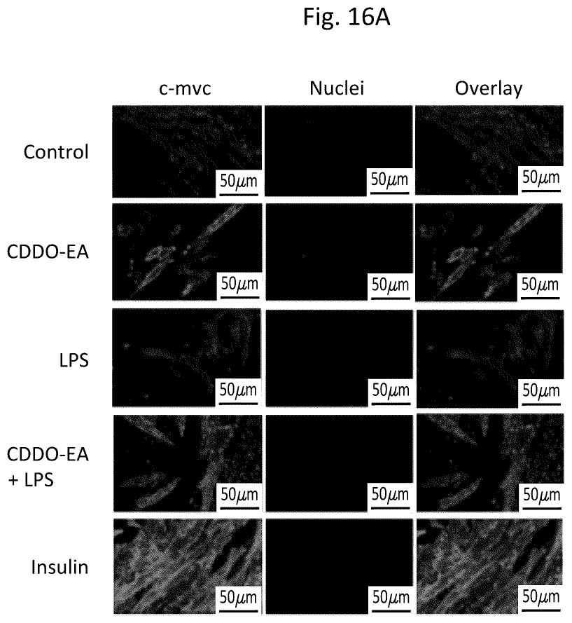

In summary, LPS stimulation of myotubes without CDDO-EA for 1 hour increased the expression levels of TNF-α and MCP-1 gene. (FIG. 15B). However, pre-treatment of myotubes with CDDO-EA for 1 h before LPS stimulation reduced LPS-induced TNF-α and MCP-1 gene expression levels.

### Example 9—CDDO-EA Induces Insulin-Independent GLUT4 Translocation in Skeletal Muscle Cells

As demonstrated herein, CDDO-EA inhibits LPS-induced inflammation in macrophages and myocytes and that the mechanism of inhibition is through NF-κB in muscle cells. The present example shows that CDDO-EA induces GLUT4 translocation and p38 phosphorylation in skeletal muscle cells. CDDO-EA is provided as a therapeutic agent and provides for a method of protecting skeletal muscle from inflammation, and inhibiting inflammation of skeletal muscle.

The CDDO-EA employed in the present studies on skeletal muscle cells was synthesized according to the methods taught by Honda et al., (1998); Honda et al., (2000b); Honda et al., 2002); Yates et al., (2007); and U.S. Pat. Nos. 6,326,507 and 6,974,801, which are all incorporated herein in their entirety.

The structure of the compound used was verified by mass spectrographic comparison with a reference structure for the Compound generated from the database, MassHunter (Agilent).

The properties of CDDO-EA in regulating glucose metabolism in skeletal muscle are examined. It was determined that CDDO-EA induced translocation of the glucose transporter, GLUT4. L6-GLUT4myc cells (rat myoblast cell line) express a c-myc epitope-tagged GLUT4 which distinguishes it from the endogenous GLUT4. This cell line is used to study GLUT4 transport in myotubes (a skeletal muscle in vitro model).

The experimental model employed in the present work are L6-GLUT4myc myoblasts. These myoblasts differentiate normally from myoblasts to myotubes, and L6-GLUT4myc cells respond to insulin with a two-fold stimulation of glucose uptake and translocation of GLUT4myc to the cell surface. In addition, L6-GLUT4myc myotubes express 100-fold more GLUT4myc than endogenous GLUT4 or GLUT1.

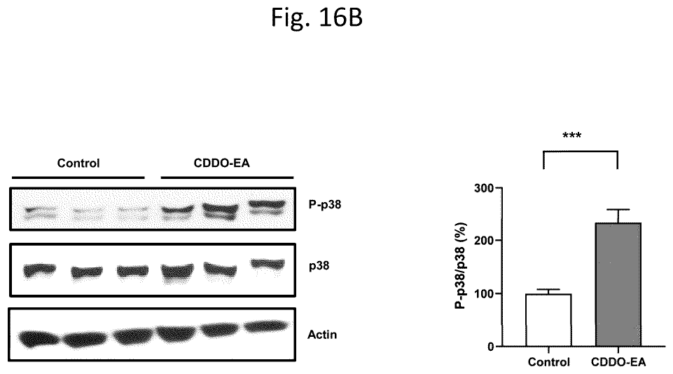

Immunofluorescence and confocal microscopy were employed to visualize the localization of c-myc epitope-tagged GLUT4 in L6-GLUT4myc myotubes treated with CDDO-EA for 1 h. L6-GLUT4myc myotubes were also treated with insulin alone for 15 min as a positive control. Insulin induced GLUT4myc translocation into the cell membrane (FIG. 16A). CDDO-EA also induced GLUT4myc translocation. LPS treatment for 1 h did not inhibit CDDO-EA-stimulated GLUT4myc translocation.

p38 has been reported to be involved in insulin-independent glucose metabolism. CDDO-EA was examined and found to activate phosphorylation of p38. These findings show that CDDO-EA was effective for inducing p38 phosphorylation in skeletal muscle cells (FIG. 16B). Therefore, CDDO-EA is established to be effective for insulin-independent glucose metabolism in skeletal muscle cells.

Detailed description: Skeletal muscle is the major site of insulin-induced glucose uptake. Insulin binds to its receptor on the skeletal muscle, initiating a signaling cascade resulting in glucose uptake. The glucose transporter, GLUT4, facilitates glucose uptake into skeletal muscle cells. The rat skeletal muscle cells (L6-GLUT4myc) express GLUT4 which is tagged with a c-myc epitope, a sequence of amino acids used for detecting the expression of recombinant proteins to distinguish them from endogenous proteins. L6-GLUT4myc cells express 100-fold more GLUT4myc than the endogenous GLUT4.

To examine the properties of CDDO-EA in regulating glucose metabolism in the skeletal muscle, the present study examined whether CDDO-EA induces translocation of GLUT4 to the cell surface (FIG. 16A). Immunofluorescence and confocal microscopy were used to visualize the localization of c-myc epitope-tagged GLUT4 in L6-GLUT4myc cells.

FIGS. 16A and 16B present the results achieved in this study. In 16A, the first row of vertical panels illustrates the localized presence of c-myc epitope-tagged GLUT4 in myotubes, in each of the 5 treatment groups (Control, CDDO-EA, LPS, CDDO-EA+LPS, Insulin) (stained in green). The second row of vertical panels (middle vertical row), shows the nuclei present in the myotubes stained in blue in each of the 5 treatment groups (Control, CDDO-EA, LPS, CDDO-EA+LPS, Insulin). The third row of vertical panels (far right vertical row) shows the merger of the first vertical row panels (far left) and the second vertical row panels (middle) for each of the treatment groups (Control, CDDO-EA, LPS, CDDO-EA+LPS, Insulin). The first horizontal row of panels (top three panels) are the Control skeletal muscle cell panels, and have not been treated with CDDO-EA or LPS. This first horizontal row of panels demonstrates the pattern of basal staining when there is no induced GLUT4myc translocation in the cells. The second horizontal row of panels (CDDO-EA) demonstrates the pattern of staining in skeletal muscle cells treated with CDDO-EA alone. The increased pattern of staining (green staining) in the CDDO-EA panels demonstrates that CDDO-EA induced GLUT4myc translocation to the cell surface, as compared to the Control group.

The third row of horizontal panels illustrates the pattern of staining in skeletal muscle cells treated with LPS. This pattern of staining demonstrates that LPS did not influence GLUT4myc translocation in the skeletal cells. The fourth row of horizontal panels illustrates the pattern of staining in skeletal muscle cells pretreated with CDDO-EA and then exposed to LPS. This fourth row of horizontal panels illustrates GLUT4myc translocation to the cell surface and demonstrates that LPS did not prevent CDDO-EA-stimulated GLUT4myc translocation to the cell surface, compared to CDDO-EA alone. The fifth row of horizontal panels represent a Positive Control group of skeletal muscle cells and illustrates the pattern of staining of the skeletal muscle cells are treated with insulin. In this row of horizontal panels, it is shown that insulin induced GLUT4myc translocation to the nucleus, compared to control group.

FIG. 16B. The protein p38 has been reported to be involved in insulin-independent glucose metabolism. FIG. 16B represents results from a study performed to illustrate that CDDO-EA activated phosphorylation of p38. Immunoblotting was performed to measure the protein content of phosphorylated p38 and total p38 protein. Protein content of actin is used as a control to confirm protein loading is the same across the lanes of the immunoblot. The pattern and intensity of the staining in each band demonstrates that the actin protein content was the same for all of the Control and CDDO-EA samples (third row of horizontal bands). The first row of horizontal bands is the Control group (bands #1, #2, and #3) content of phosphorylated p38 (P-p38) with no CDDO-EA treatment. This shows the basal protein content of p38.

The first row of horizontal bands (#4, #5 and #6) illustrates the staining pattern of skeletal muscle cells treated with CDDO-EA. The density of the staining in these bands #4-#6 illustrates that CDDO-EA stimulates p38 phosphorylation (horizontal bands #4-#6 are darker compared to horizontal bands #1-#3 (Control)). The second horizontal row of panels (middle row) illustrate the protein content of total p38 protein (no phosphorylation) in the Control and CDDO-EA skeletal cell groups, and demonstrates that CDDO-EA had no effect on p38 protein expression (all bands of staining in this middle row appear to be similar). The effect of CDDO-EA in activating p38 activation is indicated by the detection of the phosphorylation of p38. To the right of the immunoblot is a bar graph. This bar graph illustrates the ratio of phosphorylated of p38 to total p38 protein and is expressed as a percent over the control group. These results demonstrate that a two-fold increase in activation of p38 phosphorylation occurred as a result of CDDO-EA. These two methodologies show that CDDO-EA stimulates insulin-independent GLUT4 translocation to the skeletal muscle cell surface, which involves activation of p38 by CDDO-EA.

### Example 10—Food Materials and Products with CDDO Derivatives and Analogs

The present example illustrates the food materials of the disclosure that include a combination of a CDDO-EA, or other CDDO analog or derivative, in a food material suitable for oral ingestion by

The preparations and compositions, in some embodiments, comprise the CDDO analog and/or derivative, included in a veterinary or human nutritional and/or food supplement, food additive, combined food and/or nutrient product, a weight loss product, an appetite suppressant, or other nutritional support preparation, in a fat-inhibiting concentration. The food preparations and/or dietary supplements may be a fluid (such as a product suitable to drinking as a beverage) or solid (such as an animal chow feed, nutritional bar, cookie, meal substitute material, or other food product, particularly a food product considered high in fat content).

A “high fat content” is defined for purposes of the present disclosure as having at least about 20% or about 30% or more, or about 40% to about 60%, about 80%, about 90%, or about 95% calories from fat content. A “low fat” content is defined for purposes of the present disclosure as less than about 20% or 25%, or less than about 30% (20%, 10%, 5%) calories from fat content.

Weekly Consumption of CDDO-EA by a Human-On average, a mouse in the HFD+CDDO-EA group eats 17 g of chow weekly. This is based on the average weekly chow consumption by the HFD+CDDO-EA group in all six weeks of experimental feeding. The content of CDDO-EA in the High Fat chow is 400 mg per Kg of chow. Thus, to determine how much CDDO-EA is in 17 g of chow (17 grams of chow is the average amount of chow+CDDO-EA mixture consumed in one week by a mouse) the following calculation was done.

$$( 400 mg / Kg ) ( 1 / 1000 mg ) ( 1 Kg / 1000 g ) ( 17 g ) = 0.0 \; 068 mg$$

$$( 0.0068 g ) ( 1000 mg / g ) = 6.8 mg$$

Thus, an average mouse ingested about 6.8 mg of CDDO-EA as part of its weekly diet of the CDDO-EA+High Fat chow diet. Each mouse had an average weight of about 24 g (weight based on average weight of HFD+CDDO-EA group before experimental feeding).

To determine the amount of CDDO-EA consumed weekly by an average about 150 lb. to about 160 pound human, the following calculations are done:

$$1 lb . = 454 g$$

$$( 150 lb . ) ( 454 g ) = 68 , 100 g$$

$$\begin{matrix}( 24 g ) ( x ) & = ( 6.8 mg ) ( 68100 g ) \\ x & = ( 6.8 mg ) ( 68100 g ) / ( 24 g ) \\ x & = 19295 mg\end{matrix}$$

$$( 19295 mg ) ( 1000 mg / g ) = 19.3 mg CDDO - EA per week$$

Thus, it is estimated that for an average adult human (about 150 lb.), a food material (such as a food material that is higher in fat content (at least about 25 to about 35% fat from calories), or dietary supplement (dietary powder, pill, cookie, meal replacement) would be supplemented so as to provide the human subject with about 15 grams to about 70 grams of CDDO-EA or other CDDO derivative or analog, per week. (or about 3 grams daily to about 7 grams to 10 grams daily).

Other ranges of ingredient CDDO or analog, derivative, of course will be provided as may be otherwise required by the particular subject human or veterinary animal, or as otherwise may be medically advised and/or required.

By way of example, it is anticipated that the amount and/or dosage of CDDO or CDDO analog/derivative that may be used in conjunction with the food material or supplement, may vary depending on the individual needs of the human subject or veterinary animal.

For a human of about 150 pounds average weight, for example and not limitation, the amount of the CDDO, or CDDO-analog or derivative (CDDO-EA) provided weekly (every 7 days or so), would be about 15 grams to about 60 grams CDDO-EA per week, or about 20 grams to about 55 grams per week, or about 20 grams to about 50 grams per week, or other amount as may be required and/or necessitated by the particular human according to the guidance of a health care professional.

Veterinary Food Preparations/Chow: It is anticipated that the present CDDO-EA (and other CDDO supplemented materials, such as non-veterinary animal, including human, food) supplemented food formulations may be prepared for particular age-range animals or particular weight range animals, so as to prevent or inhibit excessive weight gain in these animals, and/or to assist in the maintenance of a healthy weight for the animal according to standard veterinary considerations. Addition of a CDDO-EA (or other CDDO analog or derivative) in an animal/veterinary food product that is higher in fat content (for increased palatability) will be especially advantageous for providing food preparations and supplements while assuring healthy weight maintenance.

By way of example, such a veterinary food or supplement preparation may be formulated according to routine methods known to those in the animal food industry. To this will be added a desired and/or dietary recommended amount of the CDDO-EA (or other CDDO analog or derivative) of about 3 to about 15 grams per serving. These food preparations are therefore sufficient to provide an animal or other subject with the desired dietary needs. By way of example, one dietary regimen will comprise between about 21 grams to about 100 grams (about 105 grams) of the CDDO or CDDO-EA (or other derivative or analog) over an average 7-day week feeding period.

### Example 11—Materials and Methods

The following description is provided for detail on the materials and methods employed in the in vitro, skeletal muscle (myotubules) studies and in vivo animal studies.

Reagents: CDDO-EA will be prepared as described in FIG. 19. CDDO-EA will be dissolved in DMSO (Sigma Aldrich) and stored in 40 mM stock concentrations at −80° C. until used for experiments. Vehicle controls contained concentration of 0.125% DMSO.

Cell culture: The L6-GLUT4myc rat myoblast cell line was provided from Ontario, Canada. L6-GLUT4myc myoblasts were maintained and differentiated to myotubes as previously described. L6-GLUT4myc rat myoblast cells were not used beyond passage 11.

Mouse RAW 264.7 macrophage cells were cultured as described previously. The cells were then sub-cultured using 5 ml of ice cold 5 mM EDTA in PBS in 4° C. for 20 min, tapping the flask every 5 min to lift the cell from the flask. The cell pellet was collected by centrifugation at 800 r/min for 5 min at room temperature.

THP-1 human monocytes were maintained in culture as previously described. THP-1 monocytes were sub-cultured when the cell concentration reached 8×10⁵ cells/ml. To differentiate THP-1 monocytes to macrophages, THP-1 monocytes were seeded at 1× 10⁶ cells/ml in 6-well Corning® cell culture treated dishes in growth medium+100 ng/ml PMA (phorbol 12-myristate 13-acetate) and cultured at 37° C. with 5% CO₂ incubator for 48 h to achieve differentiation to macrophages.

Western blotting and subcellular fractionation: L6-GLUT4myc myoblasts (4×10⁴ cells/ml, 2 ml/well) were seeded onto 6-well dishes and differentiated with differentiation medium for 7 days. Myotubes were treated with LPS 100 ng/ml (Escherichia coli 0111: B4, Sigma Aldrich) in the absence or presence of CDDO-EA 500 nM, as indicated. After treatment, myotubes were lysed using lysis buffer (containing 1% Triton-X) with 50 mM Hepes pH 7.6 (Sigma Aldrich), 50 mM NaCl (Sigma Aldrich), 20 mM sodium pyrophosphate (Thermo Fisher Scientific), 20 mM β-glycerophosphate (Sigma Aldrich), 10 mM NaF (Thermo Fisher Scientific), 1 mM Na₃VO₄ (Sigma Aldrich), 1 mM PMSF (Sigma Aldrich,), 1 mM aprotinin (Sigma Aldrich) and 1 mM leupeptin (Sigma Aldrich). Protein concentrations were determined using the Pierce BCA Protein Assay (Thermo Fisher Scientific), and equal amounts of protein were separated by electrophoresis and subsequently transferred to nitrocellulose membranes. Membranes were analyzed using the following antibodies from Cell Signaling Technology: phosphorylated NF-κB (Ser536) (93 H₁, cat #3033,1:1000), phosphorylated IKB-α (Ser32) (14D4, cat #2859,1:1000), IKB-α (44D4, cat #4812, 1:1000), phosphorylated p38 (Thr180/Tyr182) (cat #9211, 1:1000), p38 (cat #9212, 1:1000). Membranes were also analyzed using NF-κB p65 (C-20, cat #sc-372, 1:1000) and Lamin A/C (H-110, cat #sc-20681, 1:1000) from Santa Cruz Biotechnology and actin antibody (cat #A1978, 1:5000) from Sigma. Primary antibodies were detected using donkey anti-rabbit (cat #sc-2077, 1:5000) or donkey anti-mouse (cat #sc-2314, 1:5000) secondary antibodies conjugated to horseradish peroxidase from Santa Cruz Biotechnology. Bands were quantitated using ImageQuant (GE Healthcare). Phosphorylated protein was normalized to total protein.

For subcellular fractionation, differentiated L6-GLUT4myc myotubes were pre-treated with CDDO-EA 500 nM for 1 h follow by LPS 100 ng/ml (E. coli O111: B4, Sigma Aldrich) treatment for 1 h, and the nuclear and cytosolic fractions were isolated using the Nuclear Extraction Kit (Panomics) according to the manufacturer's instructions. The extracted fractions were then analyzed by Western blotting as described.

Immunofluorescence microscopy: L6-GLUT4myc myoblasts (4×10⁴ cells/ml, 0.2 ml/well) were plated onto Nunc Lab-TekII 8-chamber slides (Thermo Fisher Scientific) and differentiated for 7 days. Myotubes were pre-treated with CDDO-EA 500 nM for 1 h followed by LPS 100 ng/ml (E. coli O111: B4, Sigma Aldrich) treatment for 1 h. After treatment, myotubes were fixed, permeabilized, and stained with anti-NF-κB p65 primary antibody (cat #sc-372, 1:100, Santa Cruz Biotechnology) and a goat anti-rabbit secondary antibody conjugated with Alexa Fluor® 488 (cat #A32731, 1:500, Invitrogen) as previously described. For GLUT4myc cell membrane staining, myotubes were pre-treated with CDDO-EA 500 nM for 1 h followed by LPS 100 ng/ml (E. coli 0111: B4, Sigma Aldrich) treatment for 1 h. Insulin 100 nM (cat #19278, Sigma Aldrich) treatment was for 15 min. After CDDO-EA, LPS, and insulin treatment, myotubes were fixed without permeabilization and stained with anti-c-myc primary antibody (cat #C3956, 1:100, Sigma Aldrich) and a goat anti-rabbit secondary antibody conjugated with Alexa Fluor® 488 (cat #A32731, 1:500, Invitrogen) as previously described.

Luciferase assay: L6-GLUT4myc myoblasts were transfected with 2 μg of phosphorylated NF-κB-MetLuc2-Reporter plasmid as described previously. After transfection, cells were plated onto a 24-well dish (1.5×10⁵ cells/well) and pre-treated with CDDO-EA for 1 h. Myotubes were then stimulated with LPS 100 ng/ml (E. coli 0111: B4, Sigma Aldrich) for 6 h, and luciferase activity was measured using the Ready-To-Glow™ NF-κB Secreted Luciferase Reporter System as described.

Quantitative RT-PCR: L6-GLUT4myc myoblasts (4×10⁴ cell/ml, 2 ml/well) were seeded on a 6-well plate and differentiated into myo-tubes with differentiation medium for 7 days. Myotubes were pre-treated with CDDO-EA 500 nM for 1 h followed by LPS 100 ng/ml (E. coli O111: B4, Sigma Aldrich) treatment for 1 h. RNA was isolated from treated myotubes using TRIzol reagent (Ambion) according to the manufacturer's instructions. Validated RT-PCR primers specific for rat TNF-α (Assay ID: Rn99999017_m1) and rat MCP-1 (Assay ID: Rn00580555_m1), or rat GAPDH (Assay ID: Rn99999916_s1) were used to quantify mRNA levels using TaqMan RNA-to-CT 1-Step Kit (Thermo Fisher Scientific), Real-time PCR was carried out using an AB7900HT Fast Real-Time PCR System (Applied Biosystems), and MCP-1 and TNF-α levels were calculated using the ΔΔC_{T} method according to the manufacturer's instructions.

Cytokine detection: RAW 264.7 cells were grown on 24-well dishes at 1×10⁶ cells/ml, 0.5 ml/well. Cells were pre-treated with CDDO-EA 500 nM for 1 h. After 1 h, cells were treated with 100 ng/ml of LPS16,18 (E. coli O111: B4, Sigma Aldrich) for 6 h, and supernatants were collected and stored at −80° C. until analysis. TNF-α from the culture media of cells was measured and quantified using a Mouse TNF (Mono/Mono) ELISA Set (BD Bioscience) according to the manufacturer's instructions.

THP-1 monocytes were plated in 24-well plate at 15×10⁴ cells/0.5 ml/well in growth medium plus 100 ng/ml PMA into each well and cultured at 37° C. with 5% CO₂ incubator for 48 h for differentiation to macrophages. After 48 h, THP-1 macrophages were pre-treated with CDDO-EA 500 nM for 1 h followed by LPS 100 ng/ml (E. coli O111: B4, Sigma Aldrich) exposure for 6 h. Supernatants were collected and stored at −80° C. until analysis. Cytokines and chemokines were measured and quantified using a Bio-Plex Pro human cytokines group I, 17-plex ELISA Kit (Bio-Rad) according to the manufacturer's instructions.

Sulforhodamine B (SRB) cell viability assay: L6-GLUT4myc myoblasts (4000 cells/well) were seeded on a 96-well plate and differentiated into myotubes with differentiation medium for 7 days. Myotubes were then treated with doubling concentrations of CDDO-EA for 6 or 24 h. After treatment, myotubes were fixed in 10% cold trichloroacetic acid (TCA) (Sigma Aldrich), for 1 h in 4° C., followed by three washings with water. The plate was then dried, and the cells were dyed with 100 μL/well of 0.057% SRB (Sigma Aldrich) in 1% acetic acid for 30 min in room temperature. The plate was washed with 1% acetic acid five times, dried, and 200 μL/well of 10 mM unbuffered tris-base solution were added to dissolve the bound SRB. The plate was shaken for 1 h and then read on SpectraMax at 560 nm. Results were normalized to values for non-treated cells.

Statistical analysis: All values were presented as mean±SEM. Comparisons between multiple groups was assessed by One-Way ANOVA with Tukey's test. Comparisons between two groups were assessed by unpaired t-test, two-tailed. P values equal to or less than 0.05 were considered significant. Data represented as mean±SEM. * P<0.05; ** P<0.01; *** P<0.001.

### Example 12—CDDO as an Appetite Suppressant

The incorporation of CDDO into a food consumed by a subject is demonstrated to result in a significant decrease in the amount of food consumed by the subject, compared to a subject not having CDDO incorporated into their food. Therefore, the present example demonstrates the utility of the present invention for providing an appetite suppressant, especially when consumed and/or combined with a high fat containing food product.

Therefore, a preparation suitable as an appetite suppressant is provided.

The mice were maintained on one of the 4 food regiment schedules as defined in Example 2. The amount of food consumed by an animal was monitored weekly. The data collected for the amount of food/chow consumed by each mouse within a group is provided in the Table 1 (above, Example 2).

The results from this study demonstrate that the animals consumed less high-fat content food as a result of combining food with CDDO-EA. Animals that were fed on high fat content food that was not combined with CDDO-EA ate significantly more food, as demonstrated. It was observed that the mice consuming the HFD combined with CDDO-EA ate about 20% less food compared to the amount of HFD food consumed by a similar mouse to which CDDO-EA was not added.

FIG. 5 provides the data in graphic format. The raw data is provided in the following Table:

TABLE 3

| | Grams of Chow per Week Consumed by Animals | | | | | |
|---|---|---|---|---|---|---|
| Mouse Group | 1 | 2 | 3 | 4 | 5 | 6 |
| LFD | 23.2 ± 0.4 | 20.3 ± 0.2 | 21.9 ± 1.1 | 21.3 ± 0.4 | 21.8 ± 1.0 | 20.0 ± 0.9 |
| LFD + CDDO-EA | 19.2 ± 0.6 | 21.5 ± 0.6 | 21.1 ± 0.7 | 20.0 ± 0.2 | 21.3 ± 0.3 | 19.8 ± 0.4 |
| HFD | 22.1 ± 0.5 | 19.3 ± 0.5 | 19.9 ± 0.5 | 20.5 ± 0.4 | 20.2 ± 0.8 | 20.5 ± 0.6 |
| HFD + CDDO-EA | 18.0 ± 0.9 ** | 18.4 ± 1.0 | 16.8 ± 0.5 ** | 15.8 ± 0.4* * | 16.2 ± 0.5 ** | 16.6 ± 0.4* |

C57Bl6/J mice were fed low fat diet (LFD) or high fat diet (HFD) with or without CDDO-EA (diet containing 0.04% CDDO-EA) for 6 weeks (n=5-7).
Data are represented as mean ± SEM (Two-Way ANOVA, Tukey's multiple comparisons).
****p < 0.0001 HFD vs HFD + CDDO-EA at week 1 and week 4;
**p < 0.003 HFD vs. HFD + CDDO-EA at week 3;
***p = 0.0001 HFD vs HFD + CDDO-EA at week 5;
***p = 0.0002 HFD vs HFD + CDDO-EA at week 6.

FIG. 5 shows decrease in food consumption in grams.

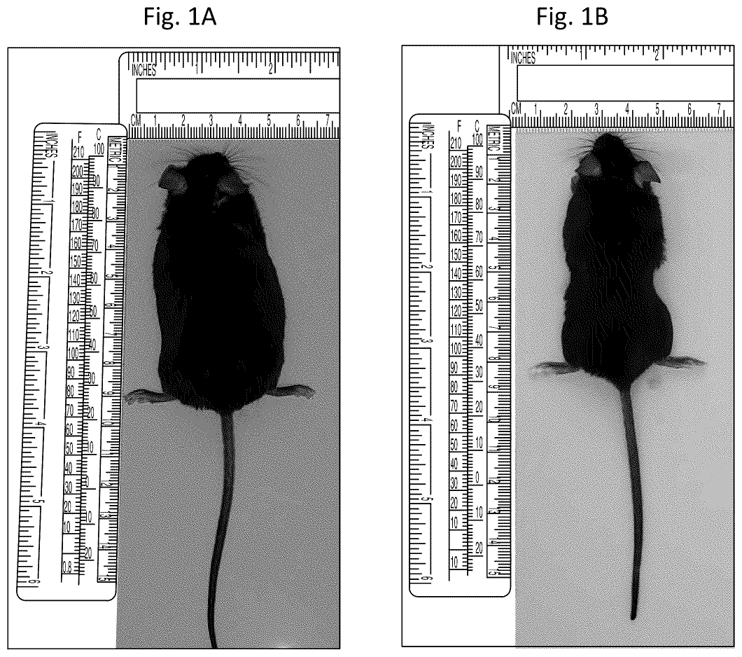

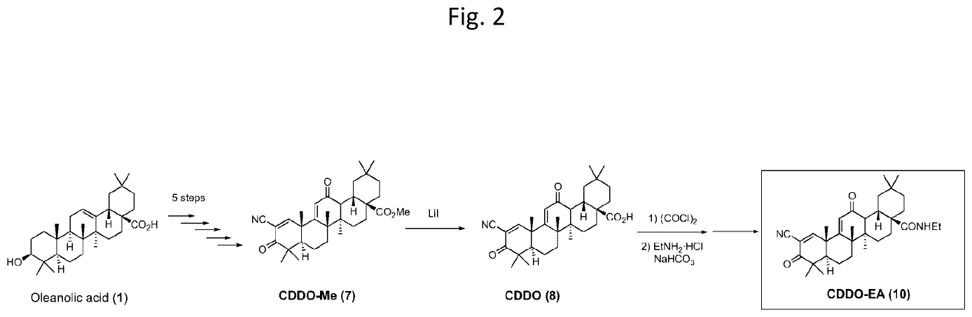

As presented in Example 2, CDDO-EA provided orally as part of a high fat containing diet, inhibits weight gain and obesity in vivo (Also see FIG. 1A (mouse fed High Fat Diet, no CDDO-EA, FIG. 1B—mouse fed High Fat Diet with CDDO-EA).

A measure of the gram amount of the HFD+CDDO-EA mouse chow consumed by an animal, compared to an animal maintained on a HFD without CDDO-EA, demonstrates that animals in the diet including the CDDO-EA consumed about 20% less food/chow, compared to the grams of chow consumed by animals on a diet of the HFD plus CDDO-EA.

The present findings demonstrate that incorporation of a CDDO (such as CDDO-EA) or CDDO derivative or analog, into food, or into the diet of an animal or other subject (human), will decrease appetite in the subject. This data demonstrates an in vivo reduction in food consumption by about a 20% amount over the course of the 6-week feeding period. The reduction in food consumption was apparent immediately in week one. Specifically, animals consuming the high fat diet (HFD) alone, at week one, were already consuming at least 10% more food per week than animals fed the high fat diet (HFD) supplemented with the CDDO-EA. By week 3, the HFD-fed animals were consuming 20% more food than the HFD plus CDDO-EA-fed animals. This decrease in food consumption continued the entire 6-week feeding period examined.

### Example 13—Weight Loss Preparations for Obese and Overweight Subject

The present example presents the preparations described herein as useful for enhancing weight loss in a subject determined to be obese (weight greater than 30% of normal average weight) or overweight (weight at least 20% greater than normal average weight).

Accordingly, a subject identified to be overweight or obese would be provided a pharmacologically effective amount of CDDO, CDDO-EA, CDDO-Me, CDDO-Im, or combination thereof on a daily basis for a sufficient period of time until a decrease in body weight is detected.

Provided as a method, a method for enhanced weight loss in an obese or overweight subject comprising: determining if a subject is obese or overweight by weighing the subject and comparing the subject weight to an average weight for a subject of similar height, age and gender; making a determination if the subject is overweight or obese and selecting an obese or overweight subject; administering providing an amount of a preparation comprising a pharmacologically effective amount of a CDDO, CDDO-EA, CDDO-Me, CDDO-Im, or combination thereof; and enhancing weight loss in the subject over a period of time compared to weight loss of a subject that is obese or overweight that is not provided the preparation.

### Example 14—NF-κB Compared to Nrf2

NF-κB is a transcription factor which regulates innate and adaptive immune functions and mediates inflammatory responses. The activation of NF-κB increases inflammation and involves the canonical pathway which responds to diverse stimuli, including ligands of several cytokine receptors and oxidative stress. In its inactive form, NF-κB is bound to IκB-α in the cytoplasm, and activation of the NF-κB results in phosphorylation and degradation of IκB-α. When NF-κB is released from I-κB-α, it translocates to the nucleus to regulate transcription of pro-inflammatory mediators, such as MCP-1 and TNF-α, and oxidative stress proteins. Deregulation of NF-κB activation contributes to the development of chronic inflammatory diseases, such as insulin resistance and type 2 diabetes.

In contrast, the transcription factor Nrf2 is responsible for protection against oxidative stress by controlling the expression of antioxidant genes (which are important in anti-inflammatory functions. Nrf2 and its negative regulator, the E3 ligase adaptor Kelch-like ECH-associated protein 1 (Keap1), play a significant role in maintaining intracellular redox homeostasis and regulating inflammation. Nrf2 regulates antioxidant encoding genes in the endogenous antioxidant system, such as the antioxidant nicotinamide adenine dinucleotide phosphate quinone oxidoreductase 1 (NQO1), antioxidant heme oxygenase1 (HO1), and glutathione S-transferase-3a (GST3α).

Fat overload in obesity leads to necrosis of adipocytes, releasing saturated fatty acids (SFA) that activate resident adipose tissue macrophages and adipocytes via the Toll like receptor 4 (TLR4). Activated macrophages and adipocytes produce pro-inflammatory mediators that recruit additional macrophages to adipose tissue. Adipocytes and adipose tissue macrophages release chemokines/cytokines into the circulation and initiate inflammation-induced insulin resistance of skeletal muscle. Published studies demonstrated that insulin resistant individuals have increased TLR4 in the skeletal muscle. Obese individuals have increased NF-κB activation and IL-6 gene expression in their skeletal muscle.

CDDO-EA is shown in the present work to inhibit weight gain. Inhibiting weight gain will reduce excess fat related activation of inflammatory responses in skeletal muscle. Improved skeletal muscle function, free of inflammation, will promote improved glucose metabolism and insulin sensitivity in these tissues, and inhibit skeletal muscle obesity-induced insulin resistance.

### Example 15—CDDO-EA Blockage of LPS-Induced MCP-1 Production, Comparison of a CDDO-EA Batch #1 and a CDDO-EA Batch #2

A second batch of CDDO-EA was synthesized as described herein (Example 17) (CDDO-EA #2). The anti-inflammatory activity of the CDDO-EA from this synthesis batch was examined using the mouse macrophage cell line RAW264.7.

The anti-inflammatory activity of a first batch of CDDO-EA (described in Example 16) (CDDO-EA #1) was also examined using the mouse macrophage cell line, RAW264.7. RAW264.7. This CDDO-EA first batch and the CDDO-EA second batch were both assessed for ability to inhibit LPS-induced production of MCP-1 in macrophages.

Macrophages were pre-treated with 500 nM CDDO-EA #1 or 500 nM CDDO-EA #2, for 1 hour. The microphages were then stimulated with 100 ng/ml of lipopolysaccharide (LPS) for 6 hr. Supernatants were collected from each culture. An enzyme linked immunoassay (ELISA) was performed to detect levels of the pro-inflammatory protein, monocyte chemotactic protein-1 (MCP-1). The macrophages were also treated with LPS alone or with CDDO-EA alone. A control (no treatment) group was also included in the study.

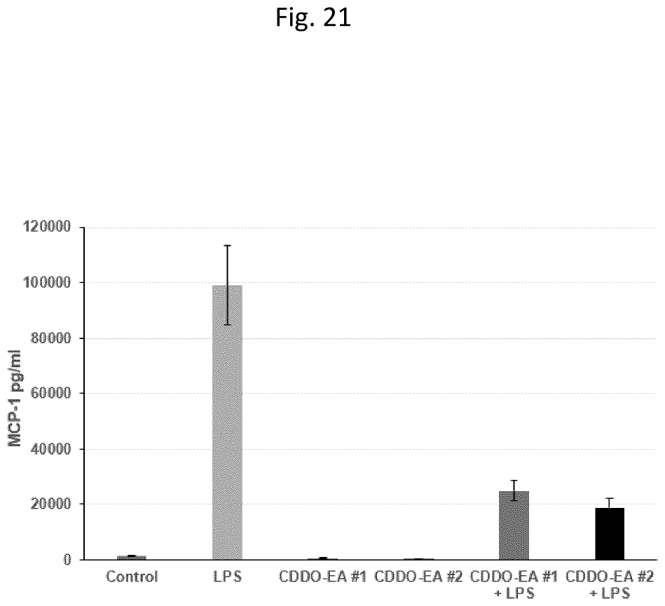

The second batch of CDDO-EA was tested for its biological property of anti-inflammatory activity. As with the first batch of CDDO-EA, this was tested by using the mouse macrophage cell line RAW264.7. RAW264.7 macrophages were pre-treated with 500 nM CDDO-EA for 1 hour then stimulated with 100 ng/ml of lipopolysaccharide (LPS) for 6 hr. Supernatants were collected, and an enzyme linked immunoassay (ELISA) was performed to detect the pro-inflammatory protein, monocyte chemotactic protein-1 (MCP-1). The macrophages were also treated with LPS alone or with CDDO-EA alone. A control (no treatment) group was also included in the study. The second batch CDDO-EA blocked the LPS-induced production of MCP-1 in RAW264.7 macrophages. This suppression of MCP-1 production by the second batch CDDO-EA is similar to that observed for the first batch of CDDO-EA. (See FIG. 21). These findings demonstrate that the second batch of CDDO-EA has anti-inflammatory properties similar to the first batch of CDDO-EA, and that the synthesis procedure provides consistent, repeatable results.

Levels of MCP-1 in the control group were similar to levels of MCP-1 in cells treated with CDDO-EA alone. The second batch of CDDO-EA (CDDO-EA #2) blocked the LPS-induced production of MCP-1 in RAW264.7 macrophages. This suppression of MCP-1 production by the second batch of CDDO-EA is similar to the level of suppression observed with the first batch of CDDO-EA. These findings show that CDDO-EA #2 elicited anti-inflammatory properties similar to CDDO-EA #1.

A comparison of this CDDO-EA #1 and the CDDO-EA #2 is shown in the data presented in the following Table 4 and Table 5 (Below).

TABLE 4

| | Control | LPS | CDDO-EA #1 | CDDO-EA #2 | CDDO-EA #1 + LPS | CDDO-EA #2 + LPS |
|---|---|---|---|---|---|---|
| | 1060 | 101949 | 717 | 481 | 30687 | 23503 |
| | 1273 | 122520 | 655 | 497 | 31996 | 25793 |
| average | 1167 | 112235 | 686 | 489 | 31341 | 24648 |
| stdev | 151 | 14545 | 44 | 11 | 926 | 1619 |
| sem | 106 | 10285 | 31 | 8 | 655 | 1145 |

TABLE 5

| Control | LPS | CDDO-EA #1 | CDDO-EA #2 | CDDO-EA #1 + LPS | CDDO-EA #2 + LPS |
|---|---|---|---|---|---|
| 1060 | 101949 | 717 | 481 | 30687 | 23503 |
| 1273 | 122520 | 655 | 497 | 31996 | 25793 |
| 1867 | 72710 | 740 | 314 | 20309 | 11890 |
| 1522 | | 870 | 587 | 17055 | 14316 |
| 1431 | 99060 | 746 | 470 | 25012 | 18875 |
| 347 | 25030 | 90 | 114 | 7448 | 6803 |
| 173 | 14451 | 45 | 57 | 3724 | 3402 |

The above data is also demonstrated in the graph at FIG. 21. This data demonstrates that the anti-inflammatory properties of the two batches of CDDO-EA #1 and #2 are similar.

### Example 16—Synthesis Procedure for CDDO-EA Batch #1

The present example details to procedure used to synthesize a first batch of CDDO-EA.

The overall yield of CDDO-EA observed was about 31%. This was accomplished in the following 8-step procedure.

### Step 1: Synthesis of methyl (4aS,6aS,6bR,8aR,10S,12aR,12bR,14bS)-10-hydroxy-2,2,6a,6b,9,9,12a-heptamethyl-1,3,4,5,6,6a,6b,7,8,8a,9,10,11,12,12a, 126, 13,14b-octadecahydropicene-4a (2H)-carboxylate (2)

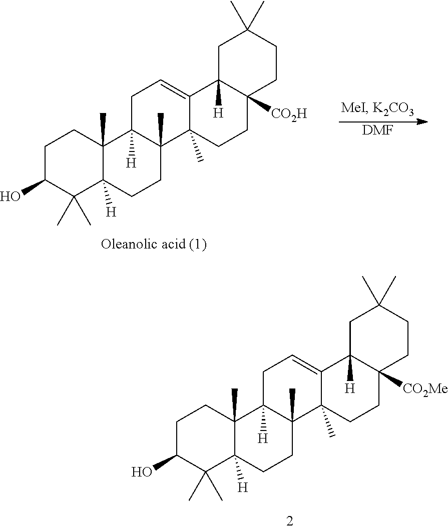

K₂CO₃ (30.0 g, 21.7 mmol, 3.0 eq) was added potion wise to a stirred solution of oleanolic acid (1) (33.0 g, 72.3 mmol, 1.0 eq) in dimethyl formamide (300 ml). The reaction mixture was cooled down to 0° C. Iodomethane (11.3 g, 4.95 ml, 79.5 mmol, 1.1 eq) was added dropwise to the suspension mixture. After the completion of addition, the reaction was warmed up to room temperature and stirred overnight. After the completion of the reaction, dimethyl formamide was removed by distillation. The resulting solid was dissolved in 1 L of dichloromethane. The solution was washed with water (300 ml) four times and sat. NaCl aq. The organic layer was dried over Na₂SO₄, and the solvent was evaporated. 32.5 g of crude product 2 was obtained (96%) as a white solid, which was used for the next step without further purifications.

### Step 2: Synthesis of methyl (4aS,6aS,6bR,8aR,12aR,12bR,14bS)-2,2,6a,6b,9,9,12a-heptamethyl-10-oxo-1,3,4,5,6,6a,6b,7,8,8a,9,10,12a, 12b, 13,14b-hexadecahydropicene-4a (2H)-carboxylate (3)

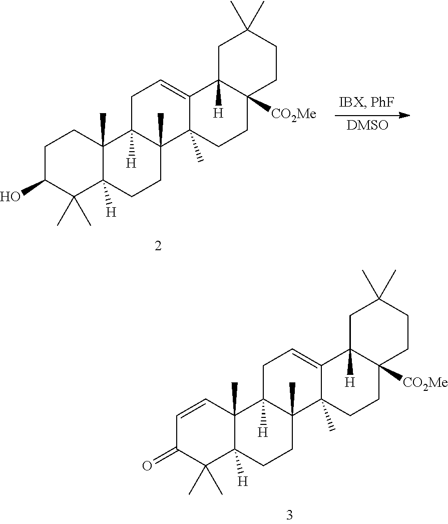

Ester 2 (29.0 g, 61.6 mmol, 1.0 eq) obtained above was dissolved in anhydrous dimethyl sulfoxide (700 ml) at room temperature. Fluorobenzene (12.3 ml) and iodoxybenzoic acid (51.7 g, 184.8 mmol, 3.0 eq) which was freshly prepared by the literature procedure2 were added to the solution. The resulting suspension was heated up to 85° C. under nitrogen and stirred for 16 hours. The reaction was cooled down and quenched with 20% sodium thiosulfate aq. (500 ml) and extracted with dichloromethane four times. The combined organic extracts were washed with sat. NaHCO₃ aq. and sat. NaCl aq. And then dried over Na₂SO₄. The solvent was removed to give the crude product 3 as yellowish solid. Purification by column chromatography (hexanes:ethyl acetate=80:20) afforded 21.4 g of white foamy compound 3 (75%).

### Step 3: Synthesis of methyl (4aR,6aR,6bR,10aR,12aR,12bS,14aS)-3,3,6b, 10,10,12a, 12b-heptamethyl-9-oxo-2,3,4,4a,6,6a,6b,9,10,10a, 11,12,12a, 126, 13,14-hexadecahydro-1H-piceno[12b, 13-b]oxirene-14a (5aH)-carboxylate (4)+methyl (4aS,6aR,6bR,8aR,12aR,12bR,14bS)-2,2,6a,6b,9,9,12a-heptamethyl-10,14-dioxo-1,3,4,5,6,6a,6b,7,8,8a,9,10,12a, 12b, 13,14,14a, 14b-octadecahydropicene-4a (2H)-carboxylate (5)

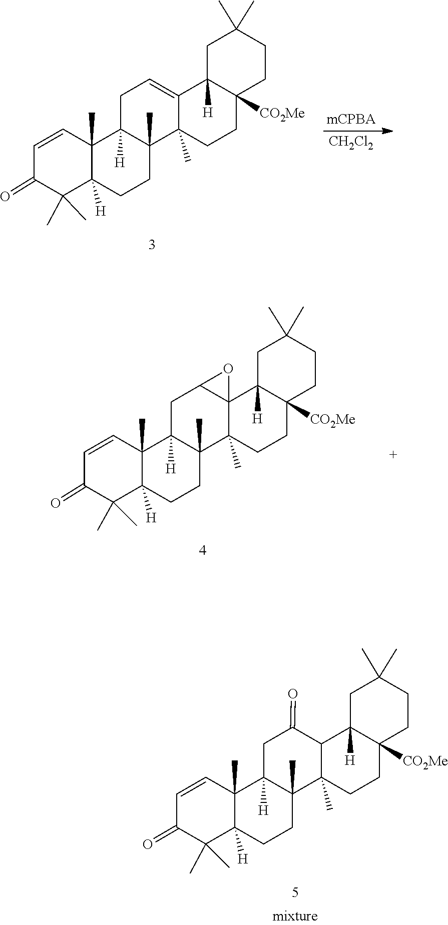

Enone 3 (21.4 g, 45.8 mmol, 1.0 equiv) was dissolved in methylene chloride (100 ml) and the solution was cooled down to 0° C. m-Chloroperbenzoic acid (14.7 grams, 70% in water, 59.5 mmol, 1.3 eq) was added potion wise at 0° C. After the completion of this addition, the reaction was warmed up to room temperature and kept stirring for overnight. The reaction mixture was diluted with methylene chloride (300 ml), and the resulting mixture was washed with 20% aqueous sodium thiosulfate three times, 10% potassium carbonate three times, and sat. NaCl aq. The organics were dried over Na₂SO₄ and the solvent was evaporated to afford 21.7 g crude mixture of 4 and 5 as white solid (98%), which was used directly for the next step without further purifications.

### Step 4: Synthesis of methyl (4aS,6aR,6bS,8aR,12aR,14bS)-11-bromo-2,2,6a,6b,9,9,12a-heptamethyl-10,14-dioxo-1,3,4,5,6,6a,6b, 7,8,8a,9,10,12a, 14,14a, 14b-hexadecahydropicene-4a (2H)-carboxylate (6)

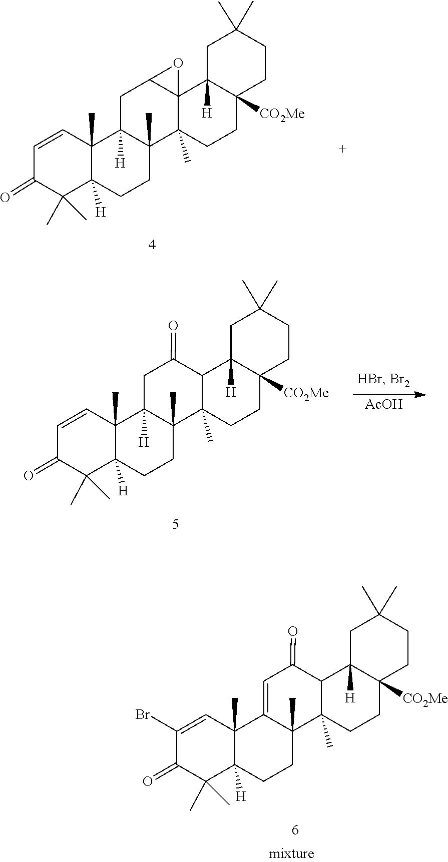

The solution of 4 and 5 (4.00 g, 8.30 mmol, 1.0 eq) in acetic acid (18 ml) was added dropwise 47% HBr aq. (0.025 ml, 0.22 mmol, 0.026 eq) at room temperature. The reaction mixture was then heated to 35° C., and Br₂ (2.13 ml, 41.3 mmol, 5.0 eq) was added dropwise. The resulting reaction mixture was kept stirring for 1.5 h at the same temperature. Acetic acid was partially removed under vacuum. The residue was then quenched with 20% aqueous sodium thiosulfate, and extracted with dichloromethane four times. The combined organic extracts were washed with saturated sodium bicarbonate twice, brine, and dried over Na₂SO₄. The solvent was evaporated, and column chromatography (hexanes:ethyl acetate=80:20) gave 3.46 g (75%) of bromo enone 6 as yellow solid.

### Step 5: Synthesis of bardoxolone methyl (CDDO-Me): methyl (4aS,6aR,6bS,8aR,12aS,14bS)-11-cyano-2,2,6a,6b,9,9,12a-heptamethyl-10,14-dioxo-1,3,4,5,6,6a,6b,7,8,8a,9,10,12a,14,14a, 14b-hexadecahydropicene-4a (2H)-carboxylate (7)

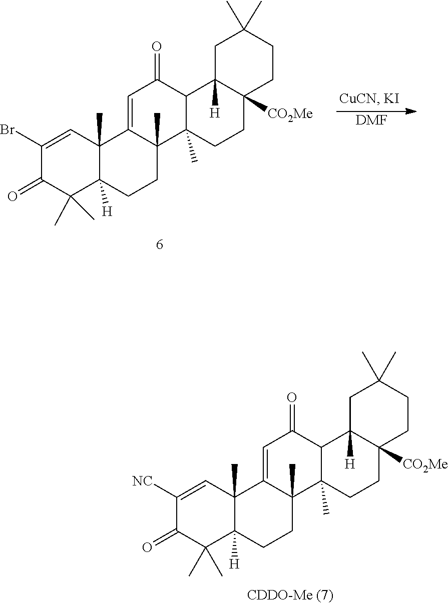

Bromo enone 6 (4.98 g mmol, 8.90 mmol 1.0 eq) is dissolved in anhydrous dimethyl formamide (50 ml) under nitrogen at room temperature. To the stirred solution, copper (I) cyanide (1.72 g, 9.79 mmol, 1.1 eq) and potassium iodide (566 mg, 1.78 mmol, 0.20 eq) were added, and the resulting reaction mixture was heated up to 120° C. and stirred for 24 h. The suspension was cooled to room temperature, quenched with water (200 ml), and diluted with ethyl acetate (500 ml). Formed cupper salts were removed by filtration before extraction. The organic phase was washed with saturated NaHCO₃ twice and sat. NaCl aq., and dried over Na₂SO₄. After evaporated the solvent, and column chromatography (hexanes:ethylacetate=75:25) to give 3.82 g (85%) of bardoxolone methyl (7) as a yellowish solid.

Step 6: Synthesis of bardoxolone (CDDO), (4aS,6aR,6bS,8aR,12aS,14bS)-11-cyano-2,2,6a,6b,9,9,12a-heptamethyl-10,14-dioxo-1,3,4,5,6,6a,6b, 7,8,8a,9,10,12a, 14,14a, 14b-hexadecahydropicene-4a (2H)-carboxylic acid (8) 3:

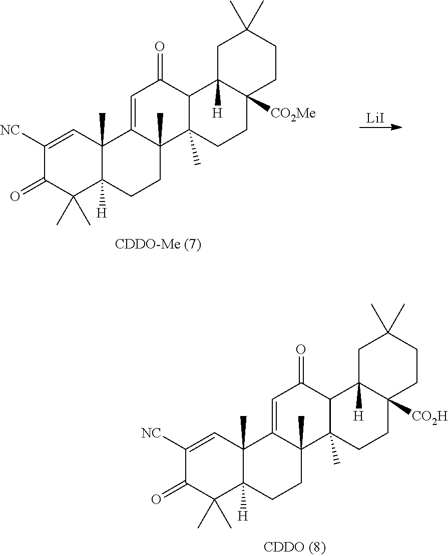

CDDO-Me (7) (3.82 g, 7.55 mmol, 1 eq) and dry LiI (18.71 g, 140 mmol, 18.5 eq) in dry DMF (10 ml) was heated under reflux for 4 h. The solution is quenched with 5% HCl aq. The mixture was extracted with EtOAc three times. The organic extract was washed with water three times, sat. NaCl aq., and dried over Na₂SO₄. The solvent was evaporated. Purification by column chromatography (dichloromethane only to dichloromethane:methanol=90:10) to give 3.56 g (96%) of CDDO (8)

### Step 7: Synthesis of 4aS,6aR,6bS,8aR,12aS,14bS)-11-cyano-2,2,6a,6b,9,9,12a-heptamethyl-10,14-dioxo-1,3,4,5,6,6a,6b,7,8,8a,9,10,12a, 14,14a, 14b-hexadecahydropicene-4a (2H)-carbonyl chloride (9) 4

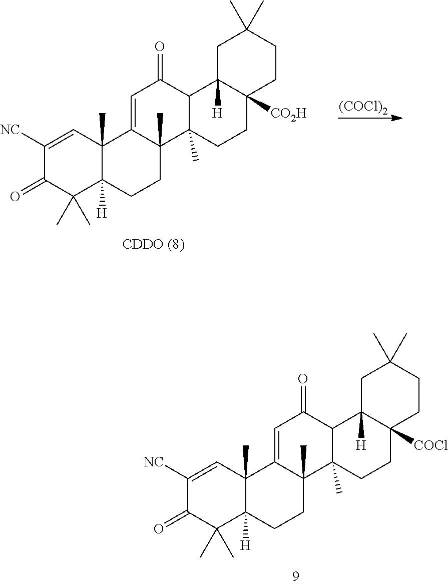

A mixture of CDDO (8) (3.0 g, 6.1 mmol) and oxalyl chloride (8.73 g, 5.9 ml, 68.8 mmlol, 11.3 eq) in anhydrous dichloromethane (60 ml) was stirred at room temperature overnight. The solvent was evaporated, and the residue was co-evaporated with benzene three times. 3.66 g (99%) of crude 9 was obtained. This was used for next step without further purification.

### Step 8: Synthesis of CDDO-EA, (4aS,6aR,6bS,8aR,12aS,14bS)-11-cyano-N-ethyl-2,2,6a,6b,9,9,12a-heptamethyl-10,14-dioxo-1,3,4,5,6,6a,6b,7,8,8a,9,10,12a, 14,14a, 14b-hexadecahydropicene-4a (2H)-carboxamide (10)

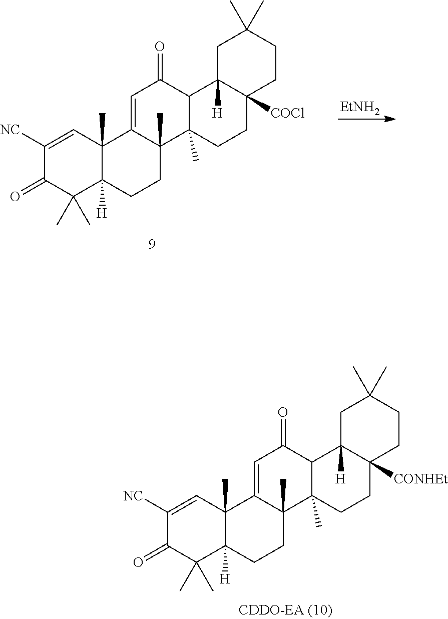

The solution of 9 (3.36 g, 6.59 mmol, 1.0 eq) in benzene (60 ml) was added to the solution of ethylamine hydrochloride (1.18 g, 14.4 mmol, 2.2 eq) and NaHCO₃ (3.0 g, 35.9 mmol, 5.5 eq) in water (60 ml). The mixture was stirred at room temperature overnight. The layers were separated, and the aqueous layer was extracted with benzene (60 ml). The combined organic layers was washed with sat. NaHCO₃aq, water, and sat. NaCl aq, and dried over Na₂SO₄. The solvent was evaporated. Purification by column chromatography (dichloromethane only to dichloromethane:methanol=90:10) to give 2.45 g (72%) of CDDO-EA (10).

The overall yield of CDDO-EA was about 31% employing the above described 8 steps.

### Example 17—Synthesis Procedure for CDDO-EA Batch #2

The present example details the procedure used to synthesize a second batch of CDDO-EA.

The overall yield observed was about 7%. This was accomplished in the following 8-step procedure.

### Step 1: Synthesis of methyl (4aS,6aS,6bR,8aR,10S,12aR,12bR,14bS)-10-hydroxy-2,2,6a,6b,9,9,12a-heptamethyl-1,3,4,5,6,6a,6b,7,8,8a,9,10,11,12,12a, 12b, 13,14b-octadecahydropicene-4a (2H)-carboxylate (2)

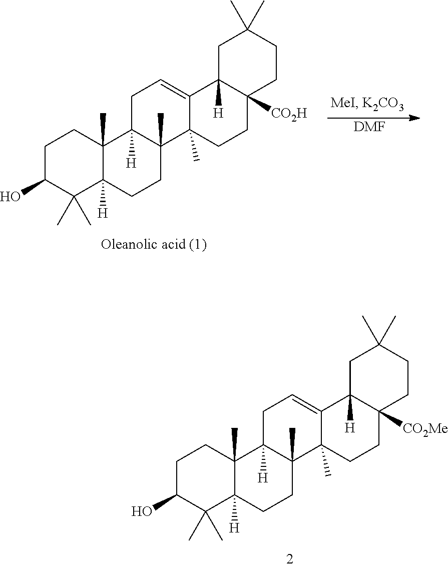

K₂CO₃ (19.1 g, 138 mmol, 3.0 eq) was added potion wise to a stirred solution of oleanolic acid (1) (21.0 g, 46.0 mmol, 1.0 eq) in dimethyl formamide (200 ml). The reaction mixture was cooled down to 0° C. Iodomethane (7.78 g, 3.15 ml, 50.6 mmol, 1.1 eq) was added dropwise to the suspension mixture. After the completion of addition, the reaction was warmed up to room temperature and stirred overnight. After the completion of the reaction, dimethyl formamide was removed by distillation. The resulting solid was dissolved in 1 L of dichloromethane. The solution was washed with water (200 ml) four times and sat. NaCl aq. The organic layer was dried over Na₂SO₄, and the solvent was evaporated. 20.3 g of crude product 2 was obtained (94%) as a white solid, which was used for the next step without further purifications.

### Step 2: Synthesis of methyl (4aS,6aS,6bR,8aR,12aR,12bR,14bS)-2,2,6a,6b,9,9,12a-heptamethyl-10-oxo-1,3,4,5,6,6a,6b,7,8,8a,9,10,12a,12b,13,14b-hexadecahydropicene-4a (2H)-carboxylate (3)

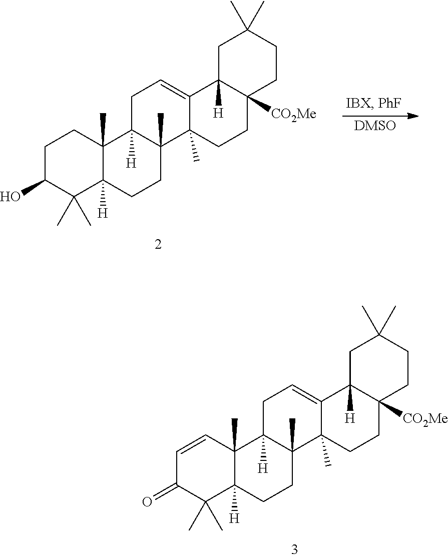

Ester 2 (20.2 g, 42.9 mmol, 1.0 eq) obtained above was dissolved in anhydrous dimethyl sulfoxide (500 ml) at room temperature. Fluorobenzene (8.6 ml) and iodoxybenzoic acid (36.0 g, 128.7 mmol, 3.0 eq) which was freshly prepared by the literature procedure were added to the solution. The resulting suspension was heated up to 85° C. under nitrogen and stirred for 18 hours. The TLC and NMR showed the mixture is the ketone intermediate, and the repeated the reaction with Fluorobenzene (8.6 ml) and iodoxybenzoic acid (36.0 g, 128.7 mmol, 3.0 eq) in 500 ml of DMSO. The 2nd TLC was the comparison of the reaction mixture and the product from the prior synthesis. This shows the major product is the compound 3. The reaction was cooled down and quenched with 20% sodium thiosulfate aq. (350 ml) and extracted with dichloromethane four times. The combined organic extracts were washed with sat. NaHCO₃ aq. and sat. NaCl aq. And then dried over Na₂SO₄. The solvent was removed to give the crude product 3 as yellowish solid. Purification by column chromatography (hexanes:ethyl acetate=80:20) afforded 11.0 g of white foamy compound 3 (55%).

### Step 3: Synthesis of methyl (4aR,6aR,6bR,10aR,12aR,12bS,14aS)-3,3,6b, 10,10,12a, 12b-heptamethyl-9-oxo-2,3,4,4a,6,6a,6b,9,10, 10a, 11,12,12a, 12b, 13,14-hexadecahydro-1H-piceno[12b,13-b]oxirene-14a (5aH)-carboxylate (4)+methyl (4aS,6aR,6bR,8aR,12aR,12bR,14bS)-2,2,6a,6b,9,9,12a-heptamethyl-10,14-dioxo-1,3,4,5,6,6a,6b,7,8,8a,9,10,12a, 12b, 13,14,14a, 14b-octadecahydropicene-4a (2H)-carboxylate (5)

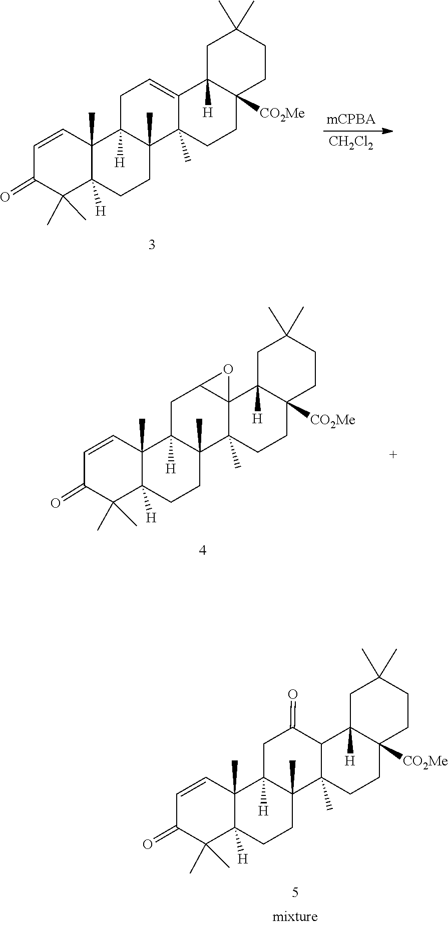

Enone 3 (10.9 g, 22.5 mmol, 1.0 equiv) was dissolved in methylene chloride (50 ml) and the solution was cooled down to 0° C. m-Chloroperbenzoic acid (7.21 grams, 70% in water, 29.2 mmol, 1.3 eq) was added potion wise at 0° C. After the completion of the addition, the reaction was warmed up to room temperature and kept stirring for overnight. The reaction mixture was diluted with methylene chloride (150 ml), and the resulting mixture was washed with 20% aqueous sodium thiosulfate three times, 10% potassium carbonate three times, and sat. NaCl aq. The organics were dried over Na₂SO₄ and the solvent was evaporated to afford 9.75 g crude mixture of 4 and 5 as white solid (90%), which was used directly for the next step without further purifications.

Step 4: Synthesis of methyl (4aS,6aR,6bS,8aR,12aR,14bS)-11-bromo-2,2,6a,6b,9,9,12a-heptamethyl-10,14-dioxo-1,3,4,5,6,6a,6b, 7,8,8a,9,10,12a, 14,14a, 14b-hexadecahvdropicene-4a (2H)-carboxylate (6):

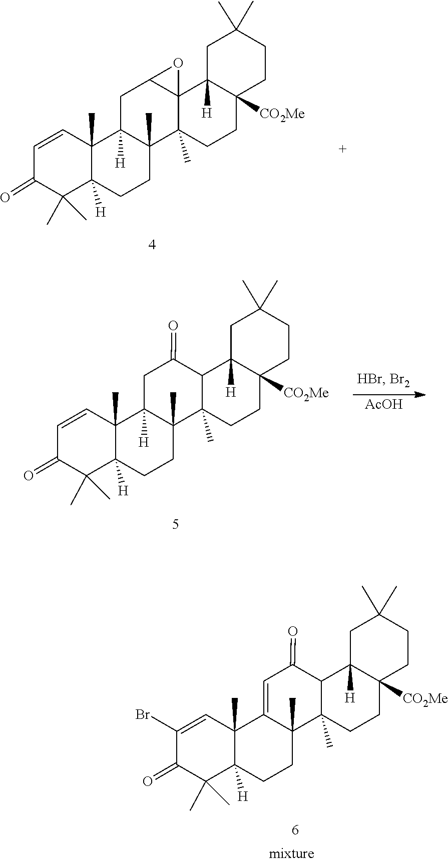

The solution of 4 and 5 (9.69 g, 20.07 mmol, 1.0 eq) in acetic acid (30 ml) was added dropwise 47% HBr aq. (0.06 ml, 0.55 mmol, 0.025 eq) at room temperature. The reaction mixture was then heated to 35° C., and Br₂ (5.17 ml, 0.1 mol, 5.0 eq) was added dropwise. The resulting reaction mixture was kept stirring for 1.5 h at the same temperature. Acetic acid was partially removed under vacuum. The residue was then quenched with 20% aqueous sodium thiosulfate, and extracted with dichloromethane four times. The combined organic extracts were washed with saturated sodium bicarbonate twice, brine, and dried over Na₂SO₄. The solvent was evaporated, and column chromatography (hexanes:ethyl acetate=80:20) gave 5.13 g (46%) of bromo enone 6 as yellow solid.

### Step 5: Synthesis of bardoxolone methyl (CDDO-Me): methyl (4aS,6aR,6bS,8aR,12aS,14bS)-11-cyano-2,2,6a,6b,9,9,12a-heptamethyl-10,14-dioxo-1,3,4,5,6,6a,6b,7,8,8a,9,10,12a, 14,14a, 14b-hexadecahydropicene-4a (2H)-carboxylate (7)

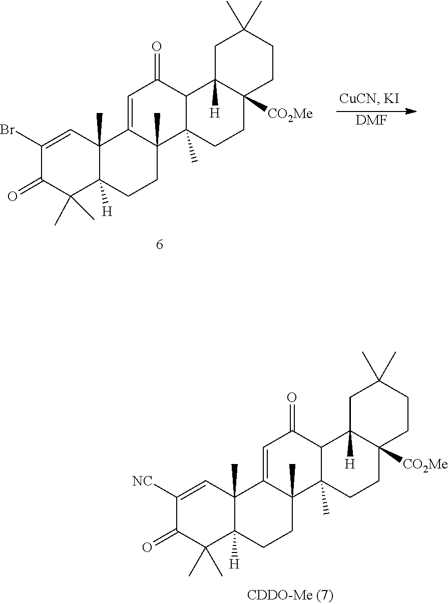

Bromo enone 6 (1.10 g, 8.57 mmol 1.0 eq) is dissolved in anhydrous dimethyl formamide (15 ml) under nitrogen at room temperature. To the stirred solution, copper (I) cyanide (193 mg, 2.16 mmol, 1.1 eq) and potassium iodide (65.4 mg, 0.394 mmol, 0.20 eq) were added, and the resulting reaction mixture was heated up to 120° C. and stirred for 9 h. TLC after 9 h showed the starting material (6) had almost disappeared, and compound (7) was formed. Since it was observed that the longer-hour reaction time made the compound (6) decompose, the reaction was stopped after 9 hours, even though unreacted starting material was left: This reaction may be time sensitive. However, 18-24 hours also worked, but 46 hours made the product (7) decompose. The suspension was cooled to room temperature, quenched with water (50 ml), and diluted with ethyl acetate (50 ml). Formed copper salts were removed by filtration before extraction. The organic phase was washed with saturated NaHCO₃ twice and sat. NaCl aq., and dried over Na₂SO₄. After evaporating the solvent, and column chromatography (hexanes:ethyl acetate=75:25), 815 mg (82%) of bardoxolone methyl (7) as a yellowish solid was produced.

### Step 6: Synthesis of bardoxolone (CDDO), (4aS,6aR,6bS,8aR,12aS,14bS)-11-cyano-2,2,6a,6b,9,9,12a-heptamethyl-10,14-dioxo-1,3,4,5,6,6a,6b, 7,8,8a,9,10,12a, 14,14a, 14b-hexadecahydropicene-4a (2H)-carboxylic acid (8) 3

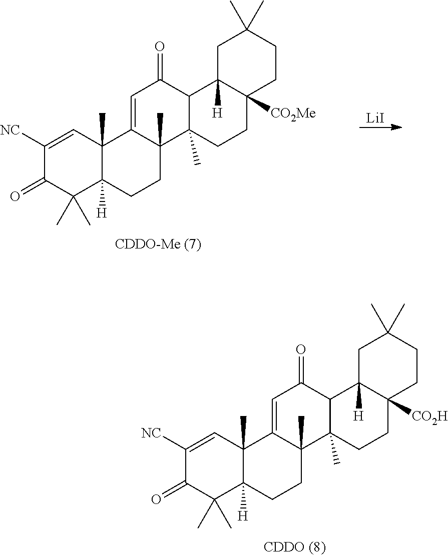

CDDO-Me (7) (800 mg, 1.58 mmol, 1 eq) and dry LiI (3.9 g, 29.3 mmol, 18.5 eq) in dry DMF (15 ml) was heated under reflux for 4 h. The solution was quenched with 5% HCl aq. The mixture was extracted with EtOAc three times. The organic extract was washed with water three times, sat. NaCl aq., and dried over Na₂SO₄. The solvent was evaporated. Purification by column chromatography (dichloromethane only to dichloromethane:methanol=90:10) was performed to give 478 mg (62%) of CDDO (8).

### Step 7: Synthesis of 4aS,6aR,6bS,8aR,12aS,14bS)-11-cyano-2,2,6a,6b,9,9,12a-heptamethyl-10,14-dioxo-1,3,4,5,6,6a,6b,7,8,8a,9,10,12a, 14,14a, 14b-hexadecahydropicene-4a (2H)-carbonyl chloride (9) 4

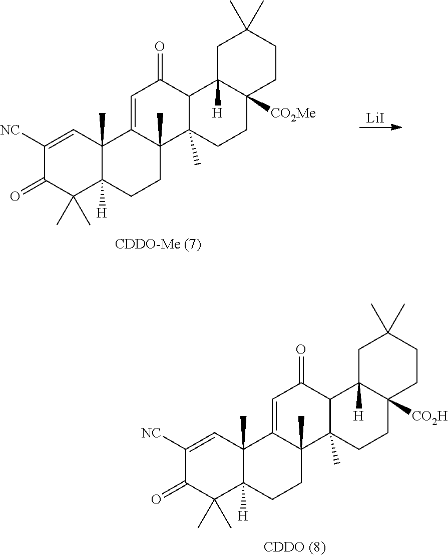

A mixture of CDDO (8) (769 mg, 1.56 mmol, 1.0 eq) and oxalyl chloride (2.24 g, 1.51 ml, 17.7 mmol, 11.3 eq) in anhydrous dichloromethane (5 ml) was stirred at room temperature overnight. The solvent was evaporated, and the residue was co-evaporated with benzene three times. 786 mg (99%) of crude 9 was obtained. This was used for next step without further purification.

### Step 8: Synthesis of CDDO-EA, (4aS,6aR,6bS,8aR,12aS,14bS)-11-cyano-N-ethyl-2,2,6a,6b,9,9,12a-heptamethyl-10,14-dioxo-1,3,4,5,6,6a,6b, 7,8,8a,9,10,12a, 14,14a, 14b-hexadecahydropicene-4a (2H)-carboxamide (10)

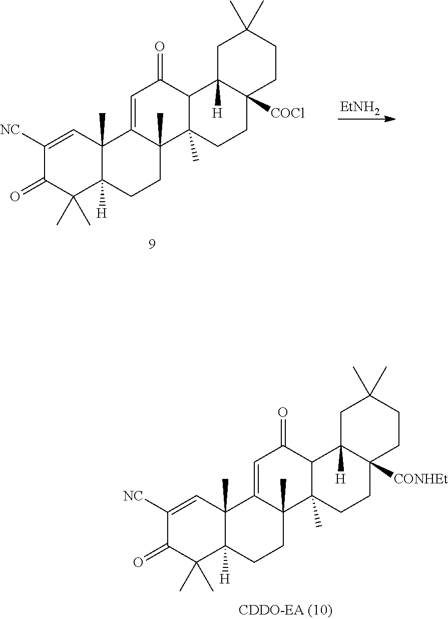

The solution of 9 (786 mg, 1.54 mmol, 1.0 eq) in benzene (15 ml) was added to the solution of ethylamine hydrochloride (285 mg, 3.89 mmol, 2.2 eq) and NaHCO₃ (690 mg, 8.47 mmol, 5.5 eq) in water (15 ml). The mixture was stirred at room temperature overnight. The layers were separated, and the aqueous layer was extracted with benzene (15 ml). The combined organic layers were washed with sat. NaHCO₃aq, water, and sat. NaCl aq, and dried over Na₂SO₄. The solvent was evaporated. Purification by column chromatography (dichloromethane only to dichloromethane:methanol=90:10) produced 521 mg (65%) of CDDO-EA (10): To obtain pure compound, the repeated column chromatography was required. The compound purity was confirmed by HPLC and NMR (>99.5%).

Overall yield of the CDDO-Me was about 7% with the above described 8 steps.

### Example 18—CDDO-EA has No Effect on Energy Expenditure

The present example demonstrates that CDDO-EA did not result in undesirable side-affects to an animal in vivo.

Shin et. al. examined the effect of a CDDO analogue on EE. Female C₅₇BL/6J mice were fed a high caloric content diet (HFD) (60% from fat caloric content) for 82 days and dosed by oral gavage with CDDO-Im (30 μmol/kg of body weight three times a week⁵. They concluded that CDDO-Im increases energy expenditure (EE) in mice fed a HFD. This interpretation of the data may be incorrect for a number of reasons. First, the interpretation included normalized body weight (they normalized to body weight). Second, the calorimetry analysis was performed after the study, i.e., when the animals were already of drastically different weights (calorimetry experiments were performed after the study when the mice were drastically different in weights.). For at least these reasons, the normalization to body weight was problematic.

In the present studies, the calorimetric study was done at the beginning of the study, thus at a time point prior to any significant divergence in body weight.

C57BL/6J mice (6-8 weeks old, male, strain #000664, The Jackson Laboratory) were divided into four (4) groups (i)-(iv)): (i) ad libitum Low fat caloric content (LFD-10% of total calories from fat caloric content, #TD.08806, Envigo), (ii) ad libitum HFD (60% of total calories from fat caloric content, #TD.06414, Envigo), (iii) HFD+CDDO-EA (diet containing 0.04% CDDO-EA), and (iv) HFD pair-fed to food intake of mice fed HFD+CDDO-EA. The study was for 6 weeks (42 days) (performed at Vanderbilt Metabolic Mouse Phenotyping Center (VMMPC)). Mice were singly housed in a Promethion Metabolic Analyzer and were monitored continuously for 7 days to assess feeding and EE.

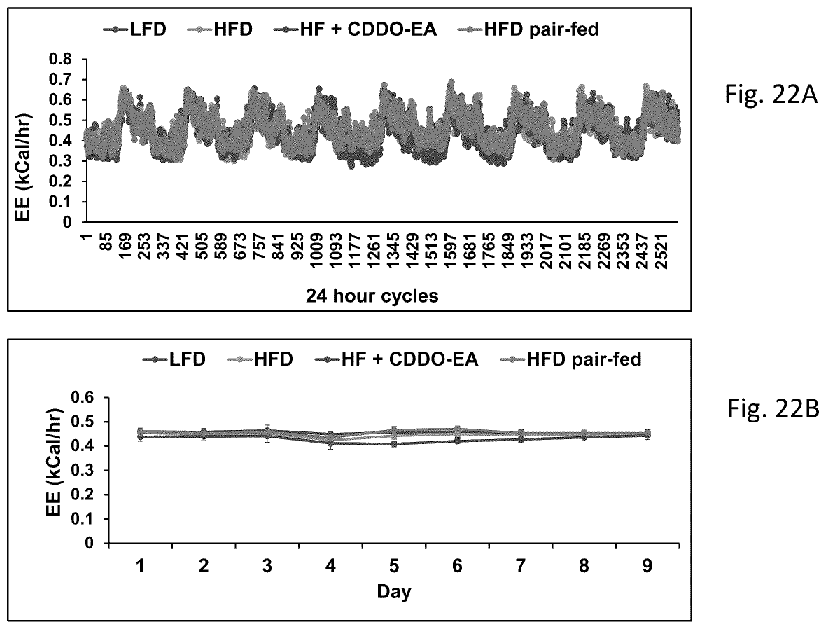

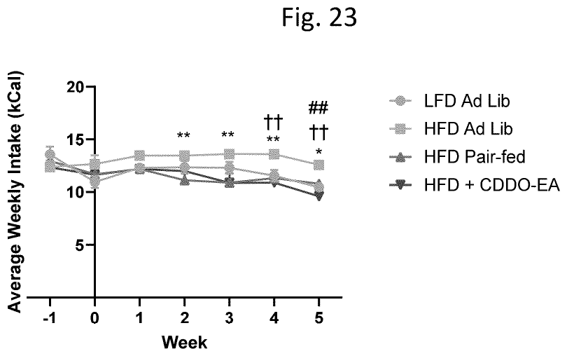

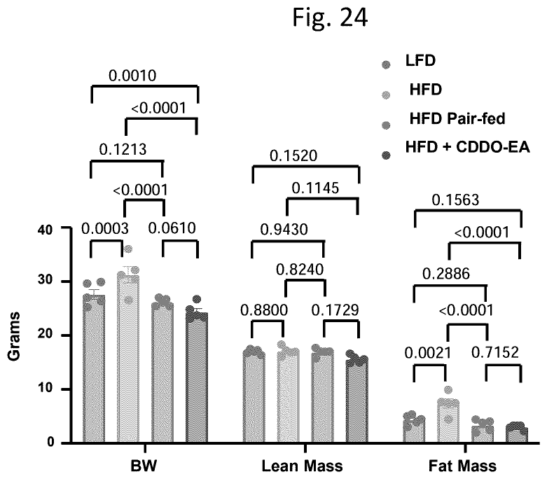

Body composition was measured by NMR. FIG. 22 shows the absolute EE (not normalized) of the four groups. The results here show that CDDO-EA does not affect EE in mice fed a high fat caloric content diet (HFD). There is no difference in EE between all groups. Also, CDDO-EA prevented an increase in food intake in HFD fed mice compared to ad libitum HFD mice (FIG. 23). The HFD pair-fed group was well paired in feeding to the HFD+CDDO-EA group. Body composition at 42 days shows no difference in lean mass between the groups (FIG. 24); thus, if EE data were to be normalized to lean mass, there would be no effect on EE by CDDO-EA. Based on these novel findings, we conclude that the ability of CDDO-EA to prevent HFD-induced obesity is due to a reduction in food intake and not an effect on EE. We hypothesize that activation of Nrf2 in the hypothalamus, a key brain region in the control of food intake, contributes to this effect of CDDO-EA.

### Example 19—Direct Metabolic Effect of CDDO-EA

In the present example, the question concerning whether the effect of CDDO-EA on glucose homeostasis is a direct effect or a secondary effect to the protection from weight gain was examined.

To determine the answer to this question, a pair-fed/weight-matched group was included in the study. OGTT was performed at end of the 6-week study.

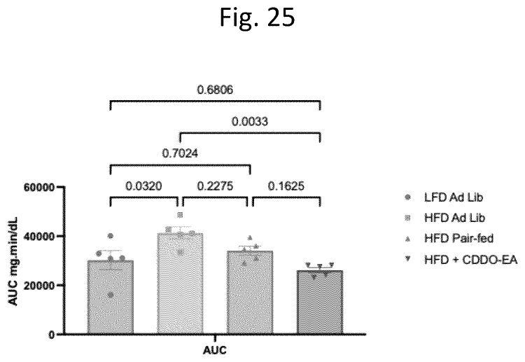

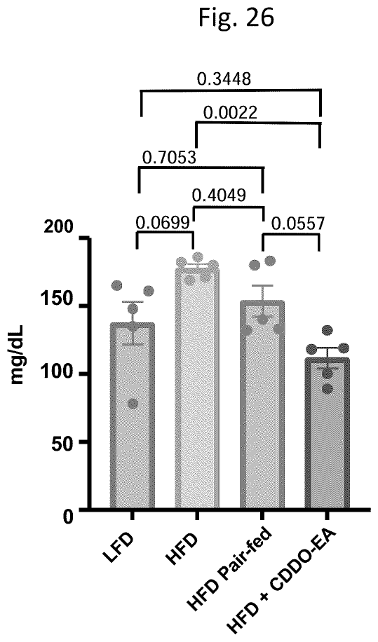

The OGTT shows a slight improvement in glucose tolerance in the HFD+CDDO-EA group when compared to the HFD pair-fed group and a significant improvement in glucose tolerance in the HFD+CDDO-EA group when compared to HFD ad lib (FIG. 25). However, there is a close to significant effect on fasting glucose in that the glucose levels are lower in the HFD+CDDO-EA group compared to the HFD pair-fed group and a significant lowered fasting glucose levels in HFD+CDDO-EA group compared to HFD ad lib group (FIG. 26). Since many factors contribute to glucose tolerance, we are proposing to test the effect of CDDO-EA on insulin action via the hyperinsulinemic-euglycemic clamp.

### Example 20—Define the Signaling Pathways Modulated by CDDO-EA to Improve Metabolism In Vitro

In the present example, the question of weather CDDO-EA (i) protection from inflammation and oxidative stress was dependent on suppression of NF-κB and activation of Nrf2 and (ii) induction of GLUT4 translocation is dependent on AMPK activation in fat, liver, and skeletal muscle cells, will be examined.

### Experimental Design

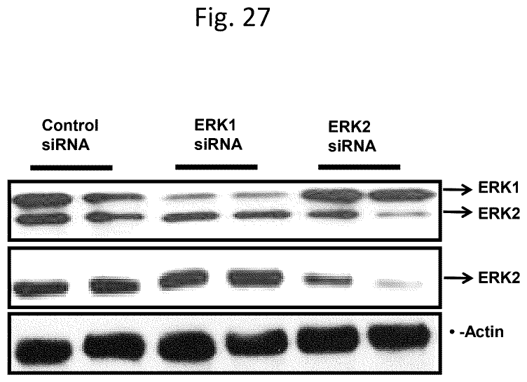

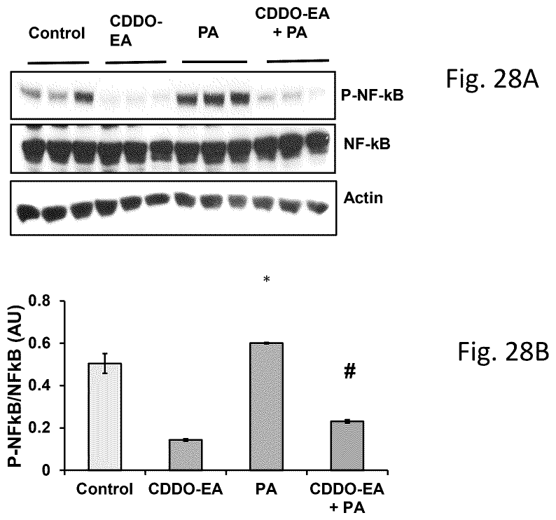

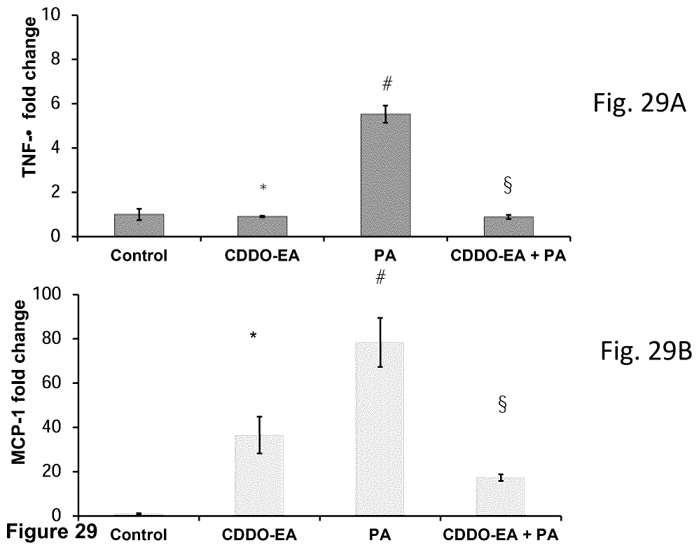

The effects of CDDO-EA on NF-κB, AMPK, and Nrf2 signaling in three cell lines will be examined, these cell lines represent three main peripheral tissues involved in glucose metabolism: 3T3-L1 (adipocytes), AML12 (hepatocytes), and L6-GLUT4myc (skeletal muscle cells). L6GLUT4myc cells were obtained from the Hospital for Sick Children at Toronto, Ontario, Canada. These cells are established in the laboratory. Published studies were previously performed using L6GLUT4myc myotubes (Chang et al., Exp. Bio. Med Maywood) 2023, 248 (2); 175-185doi 10,1177/1535702221139188. Epub 2023 Jan. 20 PMID36661241. PMCID PMC 10041051). AML12 cells will be differentiated by lowering the serum concentration in the culture medium. Authentication of these differentiated liver cells will be determined by using abundance of the enzyme carbamoyl phosphate synthase as a marker of hepatocyte differentiation. 3T3-L1 cells will be differentiated by culturing cells in medium consisting of methylisobutylxanthine, dexamethasone, and insulin, and differentiation will be authenticated by Oil Red O staining to monitor lipid accumulation or by monitoring expression of adiponectin. For FIGS. 28 and 29, the treatment profile was not followed according to Table 1. In addition, for FIGS. 28 and 29, the original CDDO-EA used was original stores provided to the lab. FIG. 27 is provided to show that the investigators were successful in the knock-down of proteins via siRNA. Extracellular signal-regulated kinase (ERK) knockdown is shown in FIG. 27. These data in FIG. 27 are separate and distinct from the CDDO-EA data presented here, and is provided for sake of completeness of overall studies conducted.

What is the effect of CDDO-EA on NF-κB activity? To address this question, the active subunit p65 of NF-κB will be knocked down (KD) by siRNA. Our laboratory is experienced with siRNA methodology; FIG. 27 shows the knockdown of extracellular signal-regulate kinase 1 and 2 (ERK1 and ERK2) in RAW264.7 cells. To mimic a diabetic environment of high glucose and high fat, cells will be treated with 25 mM glucose and 400 μM palmitate (PA) for 6 hours with pre-treatment with CDDO-EA (500 nM, 1 hour);

TABLE 5
Treatment Profile

| | | | | | | | | | | | |
|---|---|---|---|---|---|---|---|---|---|---|---|
| CDDO- | − | + | − | − | − | − | − | − | + | + | + |
| EA | | | | | | | | | | | |
| PA | − | − | − | − | − | − | + | + | + | − | + |
| Gluc | − | − | − | − | − | + | − | + | − | + | + |
| LPS | − | − | − | − | + | − | − | − | − | − | − |
| OL | − | − | − | + | − | − | − | − | − | − | − |
| Man | − | − | + | − | − | − | − | − | − | − | − |

Palmitate (PA),
Glucose (Gluc),
Oleate (OL),
Mannitol (Man)

The Table above (Table 5) shows the treatment profile. The data demonstrates (FIG. 28) that CDDO-EA inhibits PA-induced NF-κB activation and MCP-1 and TNF-α gene expression in L6GLUT4myc myotubes (FIG. 29). These data shown in FIGS. 28 and 29 did not follow the Table 1 profile. Mannitol (25 mM) and oleate (200 mM) treatments will be used as negative controls for glucose and palmitate treatments, respectively. LPS will be a positive control for NF-κB activation. MCP-1 and TNF-α gene expression will be measured by RT-PCR. Cell lysates will be collected for Western blot analysis for NF-κB pathway signaling. In addition, NF-κB activation will be assessed by measuring binding of p65 NF-κB subunit using ELISA kits. The same treatment profile followed for CDDO-EA will also be followed for CDDO-Me to compare CDDO-EA's and CDDO-Me's biological activities against NF-κB in respect to T2D. Unlike Saha et. al. (11, reported an increase in phosphorylation of IκB-α as measured by Western blotting) in only liver homogenates from mice HFD and treated with CDDO-Me4, an in vitro investigation will be utilized to assess whether CDDO-EA protection from inflammation in peripheral tissues in a diabetic environment is dependent on the inhibition of the activation of NF-κB signaling.

Expected Outcomes: In the cells without knockdown of p65 NF-κB, we expect for CDDO-EA to block PA and glucose induced activation of p65 NF-κB (via ELISA kit for p65 activation) and gene expression of MCP-1 and TNF-α in 3T3-L1 adipocytes, AML12 hepatocytes, and L6GLUT4myc myotubes. Western blot analysis will show a decrease in NF-κB and IκB-α phosphorylation. Knockdown of NF-κB will mimic the effect of CDDO-EA by inhibiting increased gene expression of MCP-1 and TNF-α. Similarly, IκB-α phosphorylation will not increase.

Pitfalls/Alternative Directions: Other pro-inflammatory pathways are also activated by LPS, PA, and glucose. In particular, the mitogen activated protein kinase (MAPK) pathway is activated downstream of Toll-like receptor 4 (TLR4) in which LPS and PA are TLR4 ligands³⁴⁻³⁶. The MAPK pathway also induces the expression of MCP-1 and TNF-α. Therefore, the activation of the MAPK pathway will be examined by measuring phosphorylation of ERK, JNK, and p38 and performing siRNA to knock down these proteins to determine if CDDO-EA protection from inflammation is dependent on the MAPK pathway. CDDO analogues have varying pharmacological efficacies on the biological effects on signaling pathways depending on the target cell type²¹; thus, rendering it possible that CDDO-EA and CDDO-Me will differentiate in their modulation of NF-κB signaling.

Is CDDO-EA-mediated GLUT4 translocation dependent on AMPK activation? The catalytic subunit alpha-1 (α1) of AMPK will be KD by siRNA. Cells will be treated with CDDO-EA (500 nM, 1 hour), insulin (100 mM for 15 min), or insulin and CDDO-EA to determine if there is a synergistic effect on GLUT4 translocation. Also, cells will be treated with the AMPK activator, 5-aminoimidazole-4-carboxamide ribonucleoside (AICAR, 1 mM, 40 minutes), as a positive control. We will use a colorimetric assay to quantitatively measure the levels of cell surface GLUT4 in 3T3-L1 adipocytes and L6GLUT4myc myotubes³⁷. FIG. 30 shows preliminary data using this colorimetry assay demonstrating that CDDO-EA and insulin induce GLUT4 translocation to the cell surface in L6GLUT4myc myotubes. GLUT4 translocation will be detected via immunofluorescence. Subcellular protein fractionation will be conducted to detect GLUT4 in the sarcolemma for myotubes and plasma membrane for adipocytes and GLUT2 in the plasma membrane of hepatocytes via Western blotting. GLUT2 will be measured in hepatocytes because GLUT2 is the most abundant glucose transporter in hepatocytes³⁸. To measure glucose uptake, a non-radioactive, plate-based bioluminescent assay (Glucose Uptake-Glo, Promega) will be performed. Using Glucose Uptake-Glo, insulin induces glucose uptake in L6GLUT4my myotubes. Phosphorylation of AMPK at Thr¹⁷² and phosphorylation of ACC at Ser⁷⁹ indicates activation of the AMPK pathway. Thus, phosphorylation of AMPK Thr¹⁷² and ACC Ser⁷⁹ will be shown through Western blotting. Further, the same tests will be performed with CDDO-Me.

Although Saha et. al. reported AMPK activation by CDDO-Me in isolated mouse hepatocytes and C2C12 muscle cells⁴, they did not examine glucose transporter translocation. Glucose transporter translocation associated with AMPK activation and glucose uptake in peripheral tissues involved in regulation of glucose metabolism. Will be more thoroughly examined.

Expected Outcomes: In cells without AMPKα1 knockdown, CDDO-EA was expected to activate AMPK indicated by increased phosphorylation of AMPK Thr¹⁷² and ACC Ser⁷⁹. CDDO-EA is expected to have a synergistic effect with insulin inducing GLUT4 translocation in L6GLUT4myc myotubes and 3T3-L1 adipocytes, and this will be associated with an increase in synergistic effect in glucose uptake. An increase in GLUT2 protein expression in the plasma membrane and immunofluorescence is expected to be associated with an increase in glucose uptake in AML12 hepatocytes. In cells with AMPKα1 knockdown, decreased phosphorylation of AMPK Thr172 and ACC Ser79 in CDDO-EA treated cells is expected. This will then be associated with a decrease in glucose uptake, decrease in GLUT4 translocation, and decrease in GLUT4 and GLUT2 protein expression and immunofluorescence. Cells with AMPKα1 knock down and without CDDO-EA treatment are expected to have baseline levels of AMPK Thr172 and ACC Ser79 phosphorylation, GLUT4 translocation, glucose uptake, and GLUT4/GLUT2 protein expression similar to cells with AMPKα1 knock down and treated with CDDO-EA. In cells with AMPKα1 knock down and treated with insulin only, an increase in GLUT4/GLUT2 protein expression and GLUT4 translocation is expected, but lower than the synergistic effect induced by CDDO-EA and insulin treatment. However, no increase in AMPKα1 Thr172 and ACC Ser79 phosphorylation above the baseline levels compared to untreated cells (no insulin and CDDO-EA) is expected. Saha et. al. showed that CDDO-Me activated AMPK signaling in isolated mouse hepatocytes and C2C12 muscle cells. Thus, it is determined here that CDDO-Me will induce the same outcomes as CDDO-EA.

Pitfalls/Alternative Directions: Knowing that analogues of CDDO have varying pharmacological efficacies on the biological effects on signaling pathways depending on the target cell type²¹, it is possible that CDDO-EA may not activate AMPK either in all the cells types or in one or two of the cell types. This could also be true for CDDO-Me in that it is not known if CDDO-Me activates AMPK in adipocytes. p38α and p38β are implicated in the regulation of insulin-independent glucose transport³⁹⁻⁴², and the present applicant has shown that CDDO-EA induces an increase in phosphorylation of p38. Thus, the knock down p38 subunits in liver and skeletal muscle cells and adipocytes will be performed to determine if GLUT4 and GLUT2 translocation is dependent on p38 activation.

Do CDDO-EA protective effects depend on Nrf2? Nrf2 will be knocked down by siRNA, and cells will be treated with the same prolife as described above. The same methodology will be used for the measurements for NF-κB and AMPK activation, MCP-1 and TNF-α expression, and GLUT4 and GLUT2 translocation. RT-PCR will be performed for measuring of Nrf2 targeted antioxidant gene expression of NQO1, HO1, and GST3α. Nrf2 nuclear translocation will also be measured.

Expected Outcomes: In L6GLUT4myc myotubes and AML12 hepatocytes with Nrf2 KD, a decrease in phosphorylation of AMPK Thr172 and ACC Ser79 may be examined. AML 12 hepatocytes with Nrf2 KD will be examined to determine if they have decreased GLUT2 translocation and uptake. We also expect L6GLUT4myc myotubes with Nrf2 KD to exhibit decreased GLUT4 translocation and uptake and AMPK activation. Similarly, we expect a decrease in AMPK activation and GLUT4 translocation and uptake in 3T3-L1 adipocytes with Nrf2 KD.

Primary cultured astrocytes from Nrf2 KO mice showed increased NF-κB activity and TNF-α expression compared to astrocytes from WT mice⁴³. Therefore, KD of Nrf2 in L6GLUT4myc, AML12 hepatocytes, and 3T3-L1 adipocytes in the present studies will be examined to determine if an increase in NF-κB activation and MCP-1 and TNF-α expression is evident. A decrease in Nrf2 targeted antioxidant gene expression of NQO1, HO1, and GST3α and no nuclear Nrf2 translocation will be examined. Conversely, in cells without Nrf2 KD, an increase in cellular GLUT4 and GLUT2 translocation and uptake and nuclear Nrf2 translocation, AMPK activation, and NQO1, HO1, and GST3α expression will e examined, and a decrease in NF-κB activation, MCP-1 and TNF-α expression will be examined.

Pitfalls/Alternative Directions: It is possible that CDDO-EA protective effects may not be dependent on Nrf2. CDDO-EA may activate other factors activated in response to inflammation and oxidative stress or involved in glucose metabolism, such as the forkhead box O (FoxO) signaling pathway^{44, 45}. Future studies could involve KD of FoxO isoforms liver and skeletal muscle cells and adipocytes.

### BIBLIOGRAPHY

The following references are incorporated herein in their entirety.
1. Cersosimo E, et al, Pathogenesis of type 2 diabetes mellitus. In: De Groot L J, et al. (eds) Endotext. South Dartmouth, MA: MDText.com, Inc., 2000.
2. Saad M J, et al, Linking gut microbiota and inflammation to obesity and insulin resistance. Physiology 2016; 31:283-93
3. Liang H, et al, Effect of lipopolysaccharide on inflammation and insulin action in human muscle. PLoS ONE 2013; 8: e63983
4. Takeda K, et al, Toll-like receptors. Ann Rev Immunol 2003; 21:335-76
5. Reyna S M, et al, Elevated toll-like receptor 4 expression and signaling in muscle from insulin-resistant subjects. Diabetes 2008; 57:2595-602
6. Wu H, et al, Skeletal muscle inflammation and insulin resistance in obesity. J Clin Invest 2017; 127:43-54
7. Reyes-Reyna S, et al, Muscle responds to an antibody reactive with the acetylcholine receptor by up-regulating monocyte chemoattractant protein 1: a chemokine with the potential to influence the severity and course of experimental myasthenia gravis. J Immunol 2002; 169:1579-86
8. Fink L N, et al, Pro-inflammatory macrophages increase in skeletal muscle of high fat-fed mice and correlate with metabolic risk markers in humans. Obesity 2014; 22:747-57
9. Sporn M B, et al, New synthetic triterpenoids: potent agents for prevention and treatment of tissue injury caused by inflammatory and oxidative stress. J Nat Prod 2011; 74:537-45
10. Tran K, et al, The combination of the his-tone deacetylase inhibitor vorinostat and synthetic triterpenoids reduces tumorigenesis in mouse models of cancer. Carcinogenesis 2013; 34:199-210
11. Saha P K, et al, The triter-penoid 2-cyano-3,12-dioxooleana-1,9-dien-28-oic-acid methyl ester has potent anti-diabetic effects in diet-induced diabetic mice and Lepr (db/db) mice. J Biol Chem 2010; 285:40581-92
12. Camer D, et al, Bardoxolone methyl prevents insulin resistance and the development of hepatic steatosis in mice fed a high-fat diet. Mol Cell Endocrinol 2015; 412:36-43
13. Stack C, et al, Triterpenoids CDDO-ethyl amide and CDDO-trifluoroethyl amide improve the behavioral phenotype and brain pathology in a transgenic mouse model of Huntington's disease. Free Rad Biol Med 2010; 49:147-58
14. Honda T, et al, A novel dicyanotriterpenoid, 2-cyano-3,12-dioxooleana-1,9 (11)-dien-28-onitrile, active at picomolar concentrations for inhibition of nitric oxide production. Bioorg Med Chem Lett 2002; 12:1027-30
15. Somwar R, et al, GLUT4 translocation precedes the stimulation of glucose uptake by insulin in muscle cells: potential activation of GLUT4 via p38 mitogen-activated protein kinase. Biochem J 2001; 359:639-49
16. Chang F M, et al, Inhibition of neddylation represses lipopolysaccharide-induced proinflammatory cytokine production in macrophage cells. J Biol Chem 2012; 287:35756-67
17. Hussey S E, et al, TAK-242, a small-molecule inhibitor of Toll-like receptor 4 signalling, unveils similarities and differences in lipopoly-saccharide- and lipid-induced inflammation and insulin resistance in muscle cells. Biosci Rep 2012; 33:37-47
18. Zhao W, et al, NF-κB- and AP-1-mediated DNA looping regulates osteopontin transcription in endotoxin-stimulated murine macrophages. J Immunol 2011; 186:3173-9
19. Hotamisligil G S, et al, Adipose expression of tumor necrosis factor-alpha: direct role in obesity-linked insulin resistance. Science 1993; 259:87-91
20. Eitas T K, et al, Differential regulation of innate immune cytokine production through pharmacological activation of Nuclear Factor-Erythroid-2-Related Factor 2 (NRF2) in burn patient immune cells and monocytes. PLoS ONE 2017; 12: e0184164
21. Li B, et al, Dihydro-CDDO-trifluoroethyl amide suppresses inflammatory responses in macrophages via activation of Nrf2. Biochem Biophys Res Commun 2014; 444:555-61
22. Ahmed H, et al, Triterpenoid CDDO-Im protects against lipopolysaccharide-induced inflammatory response and cytotoxicity in macrophages: the involvement of the NF-κB signaling pathway. Exp Biol Med 2022; 247:683-90
23. Frost R A, et al, Lipopolysaccharide regulates pro-inflammatory cytokine expression in mouse myoblasts and skeletal muscle. Am J Physiol Regul Integr Comp Physiol 2002; 283: R698-709
24. Oeckinghaus A, et al, The NF-κB family of transcription factors and its regulation. Cold Spring Harb Perspect Biol 2009; 1: a000034
25. Kanda H, et al, MCP-1 contributes to macrophage infiltration into adipose tissue, insulin resistance, and hepatic steatosis in obesity. J Clin Invest 2006; 116:1494-505
26. Jaldin-Fincati J R, et al, GLUT4 translocation in single muscle cells in culture: epitope detection by immunofluorescence. Methods Mol Biol 2018; 1713:175-92
27. Sylow L, et al, Rac1 governs exercise-stimulated glucose uptake in skeletal muscle through regulation of GLUT4 trans-location in mice. J Physiol 2016; 594:4997-500828.
28. Chambers M A, et al, Stretch-stimulated glucose uptake in skeletal muscle is mediated by reactive oxygen species and p38 MAP-kinase. J Physiol 2009; 587:3363-73
29. Konrad D, et al, The antihyperglycemic drug alpha-lipoic acid stimulates glucose uptake via both GLUT4 translocation and GLUT4 activation: potential role of p38 mitogen-activated protein kinase in GLUT4 activation. Diabetes 2001; 50:1464-71
30. Thimmulappa R K, et al, Preclinical evaluation of targeting the Nrf2 pathway by triterpenoids (CDDO-Im and CDDO-Me) for protection from LPS-induced inflammatory response and reactive oxygen species in human peripheral blood mononuclear cells and neutrophils. Antioxid Redox Signal 2007; 9:1963-70
31. Stadheim T A, et al, Eastman A. The novel triterpenoid 2-cyano-3,12-dioxooleana-1,9-dien-28-oic acid (CDDO) potently enhances apoptosis induced by tumor necrosis factor in human leukemia cells. J Biol Chem 2002; 277:16448-55
32. Yore M M, et al, The synthetic triterpenoid 1-[2-cyano-3,12-dioxooleana-1,9 (11)-dien-28-oyl]imidaz-ole blocks nuclear factor-κB activation through direct inhibition of IκB kinase beta. Mol Cancer Ther 2006; 5:3232-9
33. Catrysse L, et al, Inflammation and the metabolic syndrome: the tissue-specific functions of NF-κB. Trends Cell Biol 2017; 27:417-29
34. Libermann T A, et al, Activation of interleukin-6 gene expression through the NF-κB transcription factor. Mol Cell Biol 1990; 10:2327-34
35. Alsayed Y, et al, Activation of Rac1 and the p38 mitogen-activated protein kinase pathway in response to all-trans-retinoic acid. J Biol Chem 2001; 276:4012-9
36. Mainiero F, et al, RAC1/P38 MAPK signaling pathway controls beta1 integrin-induced interleukin-8 production in human natural killer cells. Immunity 2000; 12:7-16
37. Fu, Liangfeng, et al, Efficient and Scalable Synthesis of Bardoxolone Methyl (CDDO-Methyl Ester). Organic Letters 2013; 15-7:1622-1625
38. Honda, T, et al, Synthesis Oleanane and Ursane Triterpenoids with Modified Rings A and C: A series of Highly Active Inhibitors of Nitric Oxide Production in Mouse Macrophages. J Med Chem 2000; 43:4233-4246.
39. Hagan, C, When are mice considered old? The Jackson Library, Jackson Blog 2017. www.jax.org/news-and-insights/jax-blog/2017/november/when-are-mice-considered-old
40. Honda, T, et al, Design, Synthesis, and Biological Evaluation of Biotin Conjugates of 1-Cyano-1, 12-dioxooleana-1, 9 (11)-dien-28-oic Acid for the Isolation of the Protein Targets. J Med Chem 2004; 47:4923-4932.
41. Liu, T, et al, NF-κB signaling in inflammation. Sig Transduct Target Ther 2, 17023 (2017). https://doi.org/10.1038/sigtrans.2017.23.
41. Saha S, et al, An Overview of Nrf2 Signaling Pathway and Its Role in Inflammation. Molecules. 2020 Nov. 23; 25 (22): 5474. doi: 10.3390/molecules25225474. PMID: 33238435; PMCID: PMC7700122.
42. Hill A A, et al, A decade of progress in adipose tissue macrophage biology. Immunol Rev. 2014; 262 (1): 134-52. Epub 2014 Oct. 17. doi: 10.1111/imr.12216. PubMed PMID: 25319332; PMCID: PMC4203421.
43. Reyna S M, et al, Elevated toll-like receptor 4 expression and signaling in muscle from insulin-resistant subjects. Diabetes. 2008; 57 (10): 2595-602. Epub 2008 Jul. 18. doi: 10.2337/db08-0038. PubMed PMID: 18633101; PMCID: PMC2551667.
44. Frigerio, M, et al, Oxidation of Alcohols with o-Iodoxybenzoic Acid in DMSO, J. Org. Chem. 1999, 64, 4537.

1. An obesity inhibiting food material comprising a high fat food containing a CDDO or a CDDO derivative in a pharmacologically active amount sufficient to block weight gain, wherein weight gain upon consumption of the high fat food with the CDDO or CDDO derivative is less than weight gain upon consumption of the high fat food without the CDDO or CDDO derivative.

2. The obesity inhibiting food material of claim 1, wherein the CDDO comprises CDDO-EA.

3. The obesity inhibiting food material of claim 2, comprising a carrier material, wherein the carrier material comprises a veterinary food material, chow mix, a human food or human dietary supplement.

4. The obesity inhibiting food material of claim 3, comprising about 400 mg CDDO-EA combined with about 1 kilogram of a veterinary chow mix.

5. The obesity inhibiting food material of claim 1, comprising a human consumable food material having a fat caloric content of about 20% to about 80%.

6. A method of inhibiting weight gain in a subject on a high fat containing diet comprising:
providing the subject with a preparation comprising an additive comprising a pharmacologically effective amount of a CDDO, CDDO derivative and/or CDDO analog, or combination thereof; and
inhibiting weight of gain in the subject consuming the high fat containing diet,
wherein body weight of the subject provided the preparation comprising the additive is less than body weight of a subject having a high fat containing diet without the additive.

7. The method of claim 6, wherein the additive comprises CDDO, and the CDDO comprises CDDO-EA.

8. The method of claim 6, wherein the subject is a human or a veterinary animal.

9. A method of inhibiting weight gain in a subject consuming a high fat diet comprising:
providing the subject with a high-fat food material comprising an additive comprising a pharmacologically active amount of a CDDO, CDDO derivative and/or CDDO analog;
inhibiting weight gain in the subject associated with the high fat diet,
wherein the amount of high fat food material consumed by the subject receiving the high fat food material comprising CDDO, CDDO derivative and/or CDDO analog will be at least 50% less than the amount of high fat food material without a CDDO, CDDO derivative and/or CDDO analog consumed by the subject.

10. The method of claim 9, wherein the high fat diet comprises a food material having a caloric content that is about 50% or more total calories from fat.

11. The method of claim 9, wherein the subject is a human or a veterinary animal.

12. A method for inhibiting LPS mediated increase in skeletal muscle cytokine and chemokine levels comprising:
providing a composition comprising an NF-κB inhibiting concentration of CDDO, CDDO analog and/or CDDO derivative to skeletal muscle; and
inhibiting cytokine and chemokine gene expression and production in the skeletal muscle cells,
wherein chemokine and cytokine levels are lower in skeletal muscle in the presence of CDDO or an analog thereof, compared to cytokine and chemokine levels in skeletal muscle in the absence of CDDO, CDDO analog and/or CDDO derivative.

13. The method of claim 12, wherein the cytokines and chemokines comprise IL-1-BB, IL-6, MCP-1 and TNF-α.

14. The method of claim 12, wherein the skeletal muscle is a human skeletal muscle or a veterinary animal skeletal muscle.
